(19) United States
(12) Patent Application Publication
(10) Pub. No.: US 2007/0191420 A1
(43) Pub. Date: Aug. 16, 2007

(54) Kinase inhibitors

(75) Inventors: Dirk Casimir Maria Leysen, Lokeren (BE); Olivier Raynald Defert, Sebourg (FR); Jan Octaaf Antoon De Kerpel, Lede (BE); Eric Pierre Paul Rene Fourmaintraux, Lomme (FR); Philippe Arzel, Gent (BE); Gert Jules Hector De Wilde, Zele (BE)

Correspondence Address: FOLEY AND LARDNER LLP;SUITE 500, 3000 K STREET NW, WASHINGTON, DC, 20007, US

(73) Assignee: Devgen NV

(21) Appl. No.: 10/589,875

(22) PCT Filed: Feb. 18, 2005
(86) PCT No.: PCT/IB05/00600

§ 371 Date: Aug. 18, 2006

Related U.S. Application Data
(60) Provisional application No. 60/545,545, filed on Feb. 18, 2004.

(30) Foreign Application Priority Data
Feb. 18, 2004 (GB) 0403635.6

(51) Int. Cl. A61K 31/4439; A61K 31/44; C07D 211/72; C07D 471/04; C12N 9/99

(52) U.S. Cl. 514/300; 435/184; 514/352; 546/113; 546/312

(57) ABSTRACT

The invention provides the use of a compound or a composition comprising said compound for inhibiting the activity of at least one kinase, other than ROCK kinase, in vitro or in vivo, pharmaceutical and/or veterinary compositions comprising such compounds, medical and veterinary uses of such compounds and the compounds themselves.

### RELATED APPLICATIONS

This application claims priority under 35 U.S.C. § 119(e) from U.S. provisional Ser. No. 60/545,545, filed Feb. 18, 2004, the entire contents of which are incorporated by reference herein.

The present invention relates to improved kinase inhibitors, methods for the preparation of these inhibitors, compositions, in particular pharmaceutical containing such inhibitors, and to uses of such derivatives.

It is known in the prior art that inhibitors of certain kinases can be used in the treatment of diabetes, obesity and other metabolic diseases. Some examples of such kinases include JNK1, p38 kinase, GSK-3, IKKbeta (IKappaB kinase beta) and p70S6K.

The art also describes that several isoforms of protein kinase C (“PKC”) are associated with metabolic diseases such as diabetes and obesity. Reference is inter alia made to U.S. Pat. No. 6,376,467, U.S. Pat. No. 6,284,784, U.S. Pat. No. 6,080,784, U.S. Pat. No. 6,057,440, U.S. Pat. No. 5,962,504, WO 02/22709, WO 01/30331, WO 96/40894 and the further references cited therein.

As described in these references, there are currently 10 known isoforms of PKC, known as alpha, beta-I, beta-II, gamma, delta, epsilon, zeta, eta, iota/lambda and theta, respectively (Nishizuka, Science 258, 607-614 (1992); Selbie et al., J. Biol. Chem. 268, 24296-24302 (1993)). Based on sequence homology and biochemical properties, these PKC isozymes are generally subdivided into three groups:
(a) the group of “conventional” PKCs comprising the alpha, beta-I, beta-II and gamma isozymes, which are all regulated by calcium, diacylglycerol and/or phorbol esters;
(b) the group of “novel” PKCs comprising the delta, epsilon, theta and eta isozymes, which are all calcium-independent, but diacylglycerol- and/or phorbol ester-sensitive; and
(c) the group of “atypical” PKCs, the zeta and iota/lambda isozymes, which are insensitive to calcium, diacylglycerol and/or phorbol 12-myristate 13-acetate.

A further subgroup may be comprised of PKC mu and protein kinase D (see for example U.S. Pat. No. 6,376,467; Johannes et al, Biol. Chem. 269, 6140-6148 (1994); and Valverde et al, Proc. Natl. Acad. Sci. USA 91, 8572-8576 (1994)).

U.S. Pat. No. 6,057,440, U.S. Pat. No. 5,698,578 and U.S. Pat. No. 5,739,322 describe the use of bis indolyl maleimide compounds as specific inhibitors of PKC beta in the prevention and treatment of diabetes and diabetes-related complications. These aforementioned patent applications and patents also describe an assay that can be used to determine the specificity of a given inhibitor for one isoform of PKC compared to another (referred to in these patents as the “PKC Enzyme Assay”).

The German patent application DE 197 40 384 A1 describes that antisense oligonucleotide sequences specific for certain PKC isoforms, and in particular against the alpha, delta, epsilon and zeta isoforms, may be used in the prevention or treatment of complications associated with diabetes.

WO 01/81633 describes the association on PKC zeta with diabetes. Similarly, WO 94/18328 describes that the “atypical” PKC isozyme iota is involved in diabetes.

The link between PKC epsilon and diabetes/obesity has been established in two model systems for diabetes and obesity, viz the sand rat Psammomys and the High Fat Fed Rat. Reference is inter alia made to Shafrir et al., Annals New York Academy of Sciences 892:223-241 (1999), Donelly and Qu, Clin. Exper. Pharmacol. And Phsyiol. 25: 79-87 (1998) and Qu et al., Journal of Endocrinology 162: 207-214 (1999). The latter two references also suggest that PKC theta may be involved in diabetes and obesity

WO 00/01805 describes PKC-epsilon knock out mice. This animal model is used to demonstrate that PKC epsilon can be used as a target for drugs to reduce anxiety, modulate alcohol consumption and drug abuse, addiction, withdrawal syndrome, muscle spasms, convulsive seizures, epilepsy and to modulate the action of drugs that target the GABA-A receptor.

WO 00/01415 and U.S. Pat. No. 6,376,467 describe the use of inhibitors of PKC epsilon in the treatment of pain, in particular chronic hyperalgesia and/or inflammatory pain (reference is also made to WO 02/102232 and WO 03/89457). As examples of suitable inhibitors, both peptides as well as small molecules are mentioned. WO 97/15575 and WO 01/83449 describe modulators of PKC with specific binding activity with respect to PKC epsilon. Peptide inhibitors that provide isozyme-specific modulation of PKC (in particular of PKC gamma and PKC epsilon) are described in WO 03/089456 and WO 03/089457.

For the sequence of human PKC epsilon, reference is made inter alia made to Basta et al., Biochim. Biophys Acta, 1132 (1992), 154-160, as well as to SWISS-PROT entry Q02156 and EMBL entry X65293.

WO 03/04612 describes the use of inhibitors of PKC theta as an immunosuppressive agent (e.g. during organ transplant) and for treatment of systemic lupus erythematosus. Reference is also made to Castrillo et al., J. Exp. Med., 194, 9 (2001), p. 1231-1242, who describe that PKC epsilon plays a critical role as a mediator in signalling cascades of activated macrophages, and that the absence of PKC epsilon can compromise the successful initiation of an effective immune response against a range of bacterial pathogens.

US 2003/0134774 describes the use of inhibitors of PKC epsilon and PKC theta in inhibiting the onset of a cardiac disorder and the progression of heart failure.

For other potential uses of inhibitors of PKC and/or of specific isoforms of PKC, reference is for example made to US 2002/0164389, US 2003/0118529, US 2003/0176424, US 2003/0176423, US 2003/0166678, US 2003/0134774, US 2003/0166678, US 2003/0176424, US 2003/0199423, WO 03/82859, WO 02/103000 and WO 02/87417.

Applicant's international application PCT/EP03/14674 entitled “Kinase sequences useful for developing compounds for the prevention and/or treatment of metabolic diseases and nucleotide sequences encoding such kinase sequences” (with a filing date of Dec. 17, 2003 and invoking on the priorities of UK application 0230014.3 and U.S. provisional application 60/436,380, both of Dec. 23, 2002) describes four kinases—referred to as “JIK”, “PSK”, “TAO1” and “Q9P2I6”, respectively)—that are potential targets in metabolic disease.

The compound (R)-(+)-trans-N-(4-pyridyl)-4-(1-aminoethyl)-cyclohexanecarboxamide (Compound 18 below) is commercially available from CALBIOCHEM as an inhibitor of “rhoA-dependent coiled coil serine/threonine kinase” or “ROCK” (Compound Y-27632; Cat. No. 688000). Reference is also made to U.S. Pat. No. 4,997,834 by Muro et al.; the European application EP 0 370 498 by Muro et al.; Chitaley et al., Nat. Med., 7, 119 (2001); Narumiya et al., Methods Enzymol., 325, 273 (2000), Davies et al., Biochem. J., 351, 95 (2000); Maekawa et al., Science, 285, 895 (1999); Hirose et al., J. Cell. Biol., 141, 1625 (1999); Uehata et al., Nature, 389, 990 (1997) and Sakamoto et al., J. Pharmacol. Sci., 92, 56 (2003). However, the prior art does not disclose that this compound can be used to inhibit selectively the calcium-independent, but diacylglycerol- and/or phorbol ester-sensitive isoforms of PKC (as mentioned below), compared to other isoforms of PKC (as mentioned below).

It is a general object of the invention to provide compounds that can be used in the pharmaceutical and veterinary field, for example in the prevention and/or treatment of diseases and disorders in humans and/or animals.

It is a particular object of the invention to provide compounds that can be used in (the preparation of pharmaceutical compositions for) the treatment of metabolic diseases such as diabetes and obesity in humans.

It is another object of the invention to provide compounds that can be used to modulate, and in particular inhibit, the activity of kinases in vitro and/or in vivo.

It is a particular object of the invention to provide compounds that have improved specificity for PKC compared to other kinases.

More particularly, it is an object of the invention to provide compounds that have improved specificity for certain isoforms of PKC compared to other isoforms.

More particularly still, it is an object of the invention to provide compounds that have improved specificity for the calcium-independent, but diacylglycerol- and/or phorbol ester-sensitive isoforms of PKC (such as the delta, epsilon, theta and eta isoforms) compared to the “conventional PKCs (i.e. the alpha, beta-I, beta-II and gamma isoforms) and the “atypical” PKCs (i.e. the zeta and iota/lambda isoforms).

Other objects, aspects, embodiments, uses and advantages of the invention will become clear from the further description below.

Generally, it has now been found that the above objectives can be achieved by compounds of the invention.

### SUMMARY OF THE INVENTION

Viewed from a first aspect, the invention provides the use of a compound or a composition comprising said compound for inhibiting the activity of at least one kinase, other than ROCK kinase, in vitro or in vivo, wherein said compound is a compound of the formula (I): 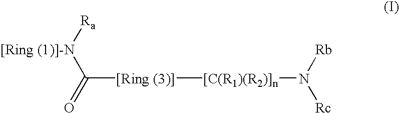
(wherein:

Ring (1) is a substituted or unsubstituted, saturated, unsaturated or aromatic 4-, 5-, 6-, 7- or 8-membered ring containing carbon atoms and at least one hydrogen-accepting heteroatom and optionally 1 or 2 further heteroatoms;

Rₐ is a hydrogen or a linear or branched, substituted or unsubstituted C₁-C₆ alkyl, substituted or unsubstituted C₁-C₆ alkoxy or substituted or unsubstituted aryl;

Ring (3) is a substituted or unsubstituted, saturated, unsaturated or aromatic 4-, 5-, 6-, 7- or 8-membered ring containing carbon atoms and optionally 1 or 2 heteroatoms;

each R₁ or R₂, may be the same or different, and is independently selected from the group consisting of hydrogen, a substituted or unsubstituted, saturated, unsaturated or aromatic 3-, 4-, 5-, 6-, 7- or 8-membered ring containing carbon atoms and optionally one or two heteroatoms, substituted or unsubstituted C₁-C₆ alkyl or cyano;

n is 0, 1 or 2; and

R_{b} and R_{c} are such that the amino group —NR_{b}R_{c} is essentially in a protonated form at a pH between 5.0-9.0;

and wherein:

(1) the group Rₐ, the nitrogen atom to which group Rₐ is bound, the carbon atom of Ring (1) to which the N—Rₐ nitrogen atom is bound, and one carbon atom of Ring (1) adjacent to the carbon atom of Ring (1) to which the N—Rₐ nitrogen atom is bound may form Ring (7) wherein Ring (7) is a substituted or unsubstituted, saturated, unsaturated or aromatic 4-, 5- or 6-membered ring that contains carbon atoms, the N—Rₐ nitrogen atom and optionally one further heteroatom chosen from oxygen, sulfur and nitrogen;

(2) where Ring (3) is a 1,4-phenylene group, one of R₁ and R₂, the carbon atom to which R₁ and R₂ are bound and two of the carbon atoms belonging to the 1,4-phenylene group may form a substituted or unsubstituted 5-, 6-, 7- or 8-membered ring that contains carbon atoms, the nitrogen atom of the amino group NR_{b}R_{c} and optionally one further heteroatom chosen from oxygen, sulfur and nitrogen and that may be saturated or contain one double bond;

(3) where Ring (3) is a 1,4-phenylene group, one of R_{b} or R_{c}, the nitrogen atom to which R_{b} or R_{c} are bound, the carbon atom to which R₁ or R₂ are bound and two of the carbon atoms belonging to the 1,4-phenylene group may form a substituted or unsubstituted 5-, 6-, 7- or 8-membered ring that contains carbon atoms, the nitrogen atom of the amino group —NR_{b}R_{c} and optionally one further heteroatom chosen from oxygen, sulfur and nitrogen and that may be saturated or contain one double bond;

(4) one of R_{b} and R_{c} may, together with the nitrogen atom of the amino group —NR_{b}R_{c}, one of R₁ and R₂ and the carbon atom to which R₁ and R₂ are bound, form a substituted or unsubstituted 5-, 6-, 7- or 8-membered ring that contains carbon atoms, the nitrogen atom of the amino group —NR_{b}R_{c} and optionally one further heteroatom chosen from oxygen, sulfur and nitrogen and that may be saturated or contain one double bond; and

(5) R_{b}, R_{c} and the nitrogen atom to which they are bound may together from a substituted or unsubstituted ring with between 3 and 10, preferably between 4 and 7, and most preferably 5 or 6 atoms in the ring (including the nitrogen atom to which both Rₐ and R_{b} are bound) so that the ring so formed consists of a nitrogen atom, carbon atoms and optionally one further heteroatom chosen from oxygen, nitrogen and sulfur; and wherein:

the distance between the at least one hydrogen-accepting heteroatom in Ring (1) and the N(Rₐ)(R_{b}) nitrogen atom, as determined using a Scatter Plot, is in the range of 11.0 to 11.8 Angstrom),

or a salt, or pro- or predrug thereof.

Viewed from a further aspect, the invention provides the use of a compound in accordance with the first aspect of the invention in the preparation of a medicament for the prevention and/or treatment of at least one disease and/or disorder selected from the group comprising metabolic diseases, anxiety, addiction, withdrawal symptoms, muscle spasms, convulsive seizures, epilepsy, pain, cardiovascular disease and heart disease; and/or for regulating the immune system and/or an immune response and/or inflammatory response in a mammal.

Viewed from a further aspect, the invention provides the use of a compound in accordance with the first aspect of the invention for:

the preparation of a medicament for the prevention and/or treatment of type II diabetes, and/or for preventing, treating and/or alleviating complications and/or symptoms associated therewith;

the prevention and/or treatment of obesity, and/or for preventing, treating and/or alleviating complications and/or symptoms associated therewith; or

the prevention, treatment and/or management of pain, and/or for preventing, treating and/or alleviating complications and/or symptoms associated therewith.

Viewed from a still further aspect, the invention provides a pharmaceutical and/or veterinary composition containing a compound in accordance with the first aspect of the invention.

Viewed from a still further aspect, the invention provides a compound in accordance with the first aspect of the invention for use in human or veterinary medicine.

Viewed from a still further aspect, the invention provides a compound in accordance with the first aspect of the invention.

### BRIEF DESCRIPTION OF THE FIGURE

FIG. 1 represents a scatter plot diagram of all the compounds showing their activity on PCK epsilon as measured in Example 4, in relation to the distance between the at least one hydrogen-accepting heteroatom in Ring (1) and the nitrogen atom of the N(R_{b})(R_{c})amino group, as determined using a Scatter Plot.

### DETAILED DESCRIPTION OF THE INVENTION

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Preferred compounds of the invention have the following characteristics or features:

(1) Ring (1) is a substituted or unsubstituted, saturated, unsaturated or aromatic 4-, 5-, 6-, 7- or 8-membered ring containing carbon atoms and at least one hydrogen-accepting heteroatom and optionally 1 or 2 further heteroatoms chosen from oxygen, sulfur and nitrogen, and in particular nitrogen;

(2) the disposition of the amide group —N(Rₐ)—C(═O)— is as disclosed in general formula (I), i.e. the nitrogen atom is attached to Ring (1) and the carbonyl carbon atom is attached to Ring (3);

(3) Ring (3) is a substituted or unsubstituted, saturated, unsaturated or aromatic 4-, 5-, 6-, 7- or 8-membered ring containing carbon atoms optionally 1 or 2 heteroatoms chosen from nitrogen, oxygen and sulfur; and

(4) n is preferably 1 or 2.

Other generally preferred features of the compounds of this invention are now set forth.

Preferably, besides the at least one hydrogen-accepting heteroatom, Ring (1) may optionally contain 2 and preferably only 1 heteroatom(s) chosen from nitrogen, oxygen and/or sulfur atoms, which 1 or 2 heteroatom(s) are preferably nitrogen. Most preferably, however, Ring (1) contains only carbon atoms and the at least one hydrogen-accepting heteroatom, and thus no further heteroatoms.

Preferably, Ring (1) may be saturated, unsaturated (i.e. containing 1 or 2 double bonds) or aromatic, and is most preferably aromatic.

Most preferably, the at least one hydrogen-accepting heteroatom in Ring (1) is a nitrogen atom.

Preferably, Ring (1) is a 5- or 6-membered ring, and more preferably a 6-membered ring. Even more preferably, Ring (1) is a 5- or 6-membered ring, and preferably a 6-membered ring, that contains carbon atoms and one hydrogen-accepting heteroatom and optionally contains 1 further heteroatom chosen from oxygen, sulfur and nitrogen, and preferably nitrogen. Most preferably, Ring (1) is a 5- or 6-membered ring, and preferably a 6-membered ring, that contains carbon atoms and the one hydrogen-accepting heteroatom, and no further heteroatoms.

Preferably, when the Ring (1) is a 5-membered ring, the at least one hydrogen-accepting heteroatom is in preferably at the 2- or the 3-position relative to the carbon atom of Ring (1) that is covalently bound to the nitrogen atom of amide group N(Rₐ)—C(═O).

When Ring (1) is a 6-membered ring, the at least one hydrogen-accepting heteroatom is preferably in the 2-, 3- or 4-position relative to the carbon atom of Ring (1) that is covalently bound to the nitrogen atom of amide group N(Rₐ)—C(═O), and most preferably in the (4)-position.

Preferably, Ring (1) may be unsubstituted or may be substituted with 1-4, and preferably 1 or 2, substitutents that are each independently chosen from the group consisting of halogen, C₁-C₆ alkyl, C₁-C₆ alkoxy, substituted or unsubstituted aryl, cyano, nitro, hydroxy and an amino group NR_{d}Rₑ (in which R_{d} and Rₑ are as defined herein). Preferably, Ring (1) is unsubstituted or is substituted with 1 or 2, and preferably only 1, such substitutent(s). These possible substitutents on Ring (1) are also generally indicated in the formulae below with “X”, it being understood that, in accordance with the foregoing, 0 or 1-4, and preferably 0, 1 or 2, and most preferably 0 or 1, such substitutents may be present, in which each time such a substitutent is present, it may be independently chosen from the group mentioned above, and it may be present on any suitable position of the ring.

According to one possible, but less preferred embodiment, Ring (1) may be substituted with a hydrogen-donating substitutent, such as —OH, —SH or most preferably an amino group —NHR_{d} (in which R_{d} is as defined herein, and is preferably an substituted or unsubstituted aryl group). This substitutent is preferably present on the carbon atom next to the hydrogen-accepting heteroatom (and when the Ring (1) is fixed with an additional Ring (7) as defined below, on the carbon atom next to the hydrogen-accepting heteroatom that is farthest removed (in terms of number of carbon atoms that lie between) from the position that the Ring (7) is attached to Ring (1).

Some preferred, but non-limiting examples of groups that may be present as Ring (1) in the compounds of the invention are: 4-pyridyl; substituted 4-pyridyl such as 2-methyl-4-pyridyl, 3-methyl-4-pyridyl, etc.; and also for example 2-arylamino-4-pyridyl.

Preferably, the invention relates to pyridinocarboxamides that can be used to modulate the activity of enzymes and/or to modulate biological processes in vitro and/or in vivo, to pharmaceutical and/or veterinary compositions that contain such derivatives, and to pharmaceutical and/or veterinary uses of such derivatives.

The invention also preferably relates to pyridinocarboxamides that can be used to modulate the activity of kinases in vitro and/or in vivo, and that as such can (also) be used to modulate the biological pathways and/or biological processes in which such kinases are involved. The pyridinocarboxamides preferably provided by this invention can also be used for preventing and/or treating diseases or disorders in which such kinases, pathways and/or processes are involved.

The use of said pyridinocarboxamides in methods for the preparation of compositions, and in particular in methods for the preparation of pharmaceutical and/or veterinary compositions is another preferred aspect of the invention.

According to a specific, but non-limiting, embodiment of the compounds of the invention, Ring (1) carries 2 substitutents on adjacent carbon atoms, which substitutents, together with the two carbon atoms of Ring (1) to which they are bound, form:

a substituted or unsubstituted, saturated, unsaturated or aromatic 4-, 5-, 6- or 7-membered ring that contains carbon atoms and at least one hydrogen donating group —(NH)— and optionally one further heteroatom chosen from oxygen, sulfur and nitrogen, and most preferably nitrogen, that is fused to Ring (1) (hereinbelow also referred to as “Ring (6)”).

When a Ring (6) is present, it is preferably a 5- or 6-membered ring, and most preferably a 5 membered ring.

When a Ring (6) is present, it preferably contains only carbon atoms and the at least one hydrogen-donating group.

When a Ring (6) is present, it may be saturated, contain 1 or 2 unsaturated bonds or be aromatic, and is preferably aromatic.

When a Ring (6) is present, the distance between the at least one hydrogen-accepting heteroatom in Ring (1) and the nitrogen atom of the at least one hydrogen donating group in Ring (6) is preferably in the range of 2.30 to 2.50 Angstrom, more preferably in the range of 2.30 to 2.45 Angstrom and most preferably in the a range of 2.30 to 2.40 Angstrom. For example, in Ring (6) shown in formula (A) below, this distance (as determined by molecular modelling using a suitable computer algorithm) is about 2.39 Angstrom, whereas in the corresponding unsaturated 5-membered ring, it is about 2.34 Angstrom, and in the corresponding unsaturated 6-membered ring, it is about 2.35 Angstrom. For a free mono-C₁-C₆alkyl amino group in the corresponding position (which is less preferred in the invention), this distance will be about 2.43 Angstrom

Ring (6) may be substituted with 1 or 2, and preferably 1, substitutent(s) that are each independently chosen from the group consisting of halogen, C₁-C₆ alkyl, C₁-C₆ alkoxy, substituted or unsubstituted aryl, nitro, hydroxy and an amino group NR_{d}Rₑ (in which R_{d} and Rₑ are as defined herein), but is preferably unsubstituted. These possible substitutents on Ring (6) are also generally indicated in the formulae below with “X”, it being understood that, in accordance with the foregoing, 0, 1 or 2, and preferably 0 or 1, such substitutents may be present, in which each time such a substitutent is present, it may be independently chosen from the group mentioned above, and it may be present on any suitable position of the ring.

Some specific, but non-limiting examples of groups that may be present as the fused bicyclic nucleus formed by Ring (1) and Ring (6) are:
a 7-azaindole group (A): 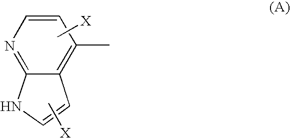
that is unsubstituted (X═H) or that be may be substituted, i.e. independently on any one of the rings or on both rings, with 1 or 2 substitutents X, in which said 1 or 2 substitutents X are independently chosen from the substitutents X as mentioned for Ring (1) and for Ring (6), respectively, hereinabove;
a 1H-pyrazolo[3,4-b]pyridine group (B): 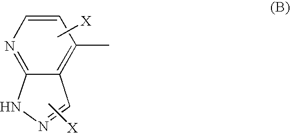
that is unsubstituted (X═H) or that be may be substituted, i.e. independently on any one of the rings or on both rings, with 1 or 2 substitutents X, in which said 1 or 2 substitutents X are independently chosen from the substitutents X as mentioned for Ring (1) and for Ring (6), respectively, hereinabove; and
a 1H-pyrazolo[3,4-b]pyridine group (C): 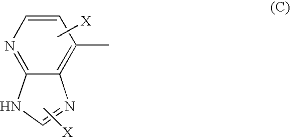
that is unsubstituted (X═H) or that be may be substituted, i.e. independently on any one of the rings or on both rings, with 1 or 2 substitutents X, in which said 1 or 2 substitutents X are independently chosen from the substitutents X as mentioned for Ring (1) and for Ring (6), respectively, hereinabove.

The amide group —N(Rₐ)—C(═O)— may have the cis-configuration or the trans-configuration, with the cis-configuration being particularly preferred.

It will also be clear to the skilled person that the amide group —N(Rₐ)—C(═O)— may be in the form of different tautomers, and all these possible tautomers are encompassed within the scope of the invention.

Also, although in the compounds of the invention the amide group —N(Rₐ)—C(═O)— is most preferably bound with its nitrogen atom to Ring (1) and with its carbon atom to Ring (3) (as shown in the compounds of general formula (I)), it is not excluded, but less preferred, that the amide group —N(Rₐ)—C(═O)— is bound with its carbon atom to Ring (1) and with its nitrogen atom to Ring (3).

The group Rₐ may be hydrogen or may be linear or branched, substituted or unsubstituted C₁-C₆ alkyl, substituted or unsubstituted C₁-C₆ alkoxy or substituted or unsubstituted aryl, and is preferably hydrogen, methyl or ethyl, with methyl and hydrogen being particularly preferred.

Alternatively, the group Rₐ, the nitrogen atom of the amide group —N(Rₐ)—C(═O)— to which said group Rₐ is bound, the carbon atom of Ring (1) to which the nitrogen atom of the amide group —N(Rₐ)—C(═O)— is bound, and one carbon atom of Ring (1) adjacent to the carbon atom of Ring (1) to which the nitrogen atom of the amide group —N(Rₐ)—C(═O)— is bound, may form a substituted or unsubstituted, saturated, unsaturated or aromatic 4-, 5- or 6-membered ring (hereinbelow also referred to as “Ring (7)”) that contains carbon atoms, the nitrogen atom of the amide group —N(Rₐ)—C(═O)— and optionally one further heteroatom chosen from oxygen, sulfur and nitrogen, and preferably nitrogen.

Ring (7) is preferably a 5- or 6-membered ring and most preferably a 5-membered ring.

Ring (7) preferably comprises carbon atoms, the nitrogen atom of the amide group —N(Rₐ)—C(═O)— and optionally one further nitrogen atom in the group Rₐ that forms the bridge between the nitrogen atom of the amide group —N(Rₐ)—C(═O)— and Ring (1), in which said nitrogen atom is preferably separated from the nitrogen atom of the amide bond in the amide group —N(Rₐ)—C(═O)— by 2 or preferably 1 carbon atoms, for example as shown the formulae below.

Ring (7) may be saturated, unsaturated and/or aromatic. When Ring (7) is a 5- or 6-membered ring, it preferably contains a double bond in the group Rₐ that forms the bridge between the nitrogen atom of the amide group —N(Rₐ)—C(═O)— and the Ring (1). More preferably, said double bond is present on the carbon atom or the nitrogen atom of the bridge Rₐ that is bound to the Ring (1), for example as shown in the formulae below.

Ring (7) may be unsubstituted or may be substituted on the group Rₐ that forms the bridge between the nitrogen atom of the amide group —N(Rₐ)—C(═O)— and the Ring (1), i.e. with one or more substitutents that are independently chosen from halogen, C₁-C₆ alkyl, C₁-C₆ alkoxy, cyano, nitro, hydroxy and an amino group NR_{d}Rₑ (in which R_{d} and Rₑ are as defined herein).

Some specific, but non-limiting examples of groups that may be present as the fused bicyclic nucleus formed by Ring (1) and Ring (7) are:
a) a which represents a 5-azaindole group (D): 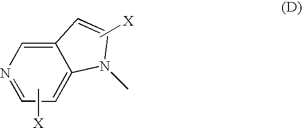
that is unsubstituted (X═H) or that be may be substituted, i.e. independently on any one of the rings or on both rings, with 1 or 2 substitutents X, in which said 1 or 2 substitutents X are independently chosen from the substitutents X as mentioned for Ring (1) and for Ring (7), respectively, hereinabove;
b) a 1H-imidazo[4,5-c]pyridine (or “5-azabenzimidazole”) group (E): 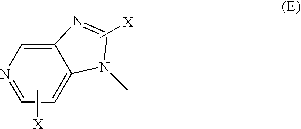
that is unsubstituted (X═H) or that be may be substituted, i.e. independently on any one of the rings or on both rings, with 1 or 2 substitutents X (and in the case of Ring (7) with only one such substitutent X), in which said 1 or 2 substitutents X are independently chosen from the substitutents X as mentioned for Ring (1) and for Ring (7), respectively, hereinabove.

The compounds of the invention may also contain both a Ring (6) and a Ring (7), that together with Ring (1) form a tricyclic ring system, in which Ring (1), Ring (6) and Ring (7) are as described herein. Some preferred, but non-limiting examples of tricyclic ring systems comprising Ring (1), a Ring (6) and a Ring (7) are:
a) a 1,6-dihydro-1,5,6-triaza-as-indacene group (F): 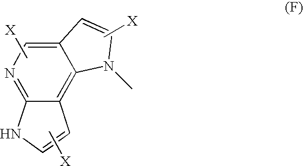
that is unsubstituted (X═H) or that be may be substituted, i.e. independently on any one of the rings, any two of the rings or on all three of the rings, with 1 or 2 substitutents X (and in the case of Ring (1) with only one such substitutent X), in which said 1 or 2 substitutents X are independently chosen from the substitutents X as mentioned for Ring (1), Ring (6) and Ring (7), respectively, hereinabove; and
b) a 1,6-dihydro-1,3,5,6-tetra-aza-as-indacene group (G): 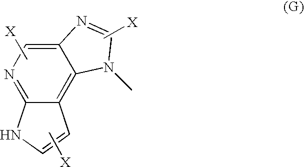
that is unsubstituted (X═H) or that be may be substituted, i.e. independently on any one of the rings, any two of the rings or on all three of the rings, with 1 or 2 substitutents X (and in the case of Ring (1) and Ring (7) with only one such substitutent X), in which said 1 or 2 substitutents X are independently chosen from the substitutents X as mentioned for Ring (1), Ring (6) and Ring (7), respectively, hereinabove.

Thus, in one embodiment, the compounds of the invention contain a bicyclic nucleus comprised of Ring (1) and a Ring (6), in which said Ring (1) and Ring (6) are as further defined herein. In such a bicyclic nucleus, either of Ring (1) and Ring (6) may be aromatic, or Rings (1) and (6) may together form an aromatic bicyclic nucleus.

In another embodiment, the compounds of the invention contain a bicyclic nucleus comprised of Ring (1) and a Ring (7), in which said Ring (1) and Ring (7) are as further defined herein. In such a bicyclic nucleus, either of Ring (1) and Ring (7) may be aromatic, or Rings (1) and (7) may together form an aromatic bicyclic nucleus.

In yet another embodiment, the compounds of the invention contain a tricyclic nucleus comprised of Ring (1), a Ring (6) and a Ring (7), in which said Ring (1), said Ring (6) and said Ring (7) are as further defined herein. In such a bicyclic nucleus, each of Ring (1), Ring (6) and Ring (7) may be aromatic, or Rings (1) and (6) may together form an aromatic bicyclic nucleus, or Rings (1) and (7) may together form an aromatic bicyclic nucleus, or Rings (1), (6) and (7) may together from an aromatic tricyclic nucleus.

Preferably, the compounds of the invention contain only a Ring (1), or a Ring (1) and a Ring (6), but no Ring (7).

Ring (3) is preferably is a 5- or 6-membered ring containing carbon atoms and optionally 1 or 2, and preferably 1, heteroatoms chosen from nitrogen, oxygen and sulfur. More preferably, Ring (3) is a 5- or 6-membered ring containing only carbon atoms.

Ring (3) is may be saturated, contain 1 or 2 unsaturated bonds, or may be aromatic, with saturated and aromatic rings being particularly preferred.

As indicated above, Ring (3) is connected to the carbon atom of the amide group —N(Rₐ)—C(═O)—, and also carries the group [C(R₁)(R₂)]ₙ—N(R_{b})R_{c}). When the Ring (3) is a 5-membered ring, the group [C(R₁)(R₂)]ₙ—N(R_{b})R_{c}) is preferably in the 3-position or the 4-position relative to the carbon atom of Ring (3) that is bound to the carbon atom of the amide group —N(Rₐ)—C(═O)—. When the Ring (3) is a 6-membered ring, the group [C(R₁)(R₂)]ₙ—N(R_{b})R_{c}) is preferably in the 3-, 4- or 5-position relative to the carbon atom of Ring (3) that is bound to the carbon atom of the amide group —N(Rₐ)—C(═O)—, and most preferably in the 4-position. However, as will be clear from the above, the invention generally comprises all isomers with respect to the positions of the amide group —N(Rₐ)—C(═O)— and the group [C(R₁)(R₂)]ₙ—N(R_{b})R_{c}) on the Ring (3), as long as in the final molecule according to Formula (I), the distance between the at least one hydrogen-accepting heteroatom in Ring (1) and the nitrogen atom of the amino group in the group [C(R₁)(R₂)]ₙ—N(R_{b})R_{c}) is in the range and preferred subranges indicated herein.

It will be clear to the skilled person that when Ring (3) is a saturated ring, the ring may be in the form of different stereoisomers with respect to the way the amide group —N(Rₐ)—C(═O)— and the group [C(R₁)(R₂)]ₙ—N(R_{b})R_{c}) are bound to said Ring (3), i.e. as cis- and trans-isomers. Both are included within the scope of the invention, with the trans-isomer being particularly preferred.

It will also be clear to the skilled person that when Ring (3) is a saturated ring that contains one or more substitutents, Ring (3) may contain one or more chiral carbon atoms and may thus exist as different isomers, e.g. enantiomers or diastereomers. All such isomers are included within the scope of the invention.

In the compounds of the invention, Ring (3) is may be unsubstituted or substituted with 1-4, preferably 1 or 2, substitutents independently chosen from halogen, C₁-C₆ alkyl, C₁-C₆ alkoxy, substituted or unsubstituted aryl, cyano, nitro, hydroxy and an amino group NR_{d}Rₑ (in which R_{d} and Rₑ are as defined herein). These possible substitutents on Ring (6) are also generally indicated in the formulae below with “Y”, it being understood that, in accordance with the foregoing, 0, or 1-4, and preferably 0, 1 or 2, such substitutents may be present, in which each time such a substitutent is present, it may be independently chosen from the group mentioned above, and it may be present on any suitable position of the ring.

Some specific, but non-limiting examples of groups that may be present as the Ring (3) are cyclopentylene, cyclopentenylene, cyclohexylene, cyclohexenylene, cyclohexdienylene and phenylene, which are connected to the amide group —N(Rₐ)—C(═O)— and the group [C(R₁)(R₂)]ₙ—N(R_{b})R_{c}) as indicated above and which may be unsubstituted or substituted with 1 or 2 substitutents independently chosen from halogen, C₁-C₆ alkyl, C₁-C₆ alkoxy, substituted or unsubstituted aryl, cyano, nitro, hydroxy and an amino group NR_{d}Rₑ (in which R_{d} and Rₑ are as defined herein).

Accordingly, some examples of Ring (3) include, but are not limited to, 1,3-cyclopentylene; 1,4-cyclopent-2-enylene; 1,3- and in particular 1,4-cyclohexylene; 1,3-1,4- or 1,5-cyclohex-2-enylene; 1,3-, 1,4- or 1,5-cyclohex-3-enylene; 1,3-, 1,5- and in particular 1,4-cyclohex-2,5-dienylene, and 1,3- and in particular 1,4-phenylene; of which 3-cyclopentylene; 1,3- and 1,4-cyclohexylene; and 1,3- and 1,4-phenylene are preferred, and 1,4-cyclohexylene and 1,4 phenylene are most preferred (and in which the numbers refer to the positions on which the —N(Rₐ)—C(═O)— and the group [C(R₁)(R₂)]ₙ—N(R_{b})R_{c}) are bound to Ring (3), respectively).

“n” in the group [C(R₁)(R₂)]ₙ—N(R_{b})R_{c}) may be 0, so that this group, an amino group —NR_{b}R_{c}; or may be 2, so as to form an ethylene amino group of the formula —(CR₁R₂—CR₁R₂)—NR_{b}R_{c}; as long as (in both cases) in the final molecule according to Formula (I), the distance between the at least one hydrogen-accepting heteroatom in Ring (1) and the nitrogen atom of the amino group in group [C(R₁)(R₂)]ₙ—N(R_{b})R_{c}) is in the range indicated above.

However, n is preferably 1, so as to form a methyleneamino group of the formula —CR₁R₂—NR_{b}R_{c}.

In the group [C(R₁)(R₂)]ₙ—N(R_{b})R_{c}), each time a group R₁ or R₂ is present, said group may be the same or different and may be independently chosen from the group consisting of: hydrogen, a substituted or unsubstituted, saturated, unsaturated or aromatic 3-, 4-, 5-, 6-, 7- or 8-membered ring containing carbon atoms and optionally one or two heteroatoms, C₁-C₆ alkyl, cyano; with hydrogen, substituted or unsubstituted C₁-C₆ alkyl and substituted or unsubstituted aryl being preferred. In particular, each R₁ and R₂ are independently chosen from the group consisting of hydrogen, methyl or ethyl. For example, when one of R₁ and R₂ is hydrogen, the other may be a methyl or ethyl.

It will be clear to the skilled person that when R₁ and R₂ are different, the compounds of the invention may exist as different isomers, e.g. enantiomers or diastereomers. All such isomers are included within the scope of the invention.

The amino group —NR_{b}R_{c} is such that, at a pH in the range of 5.0-9.0, preferably 6.0-8.0, such as pH about 7.0, it is essentially in a protonated form. Essentially in a protonated form herein generally means that at least 50%, preferably at least 75%, more preferably at least 90%, even more preferably at least 95% of all amino groups are protonated at the pertinent pH. Whether or not an amino group —NR_{b}R_{c} is essentially in a protonated form at a pH in the range above may be calculated using a suitable computer algorithm or may be determined experimentally using a technique known per se for determining the pKₐ.

R_{b} and R_{c} may be the same or different and are preferably independently chosen from the group consisting of hydrogen, substituted or unsubstituted C₁-C₁₀, preferably C₁-C₆ alkyl, still more preferably C₁-C₄ alkyl, such as C₁, C₂ and/or C₃ alkyl, such as methyl, ethyl, i-propyl and n-propyl.

Accordingly, some particular, but non-limiting examples of the group —NR_{b}R_{c} are: amino, methylamino, ethylamino, n-propylamino, i-propylamino, n-butylamino, i-butylamino, t-butylamino, dimethylamino, ethylmethylamino, methyl-n-propylamino, methyl-i-propylamino, n-butylmethylamino, i-butylmethylamino, t-butylmethylamino, diethylamino, ethyl-n-propylamino, ethyl-i-propylamino, n-butylethylamino, i-butylethylamino, t-butylethylamino, di-n-propylamino, di-i-propylamino, di-n-butylamino, di-i-propylamino, di-t-butylamino, as well as mono- or dialkylamino groups in which one or both of the alkyl groups contain more than 4 carbon atoms, such as the various isomers of pentylamino, hexylamino, heptylamino, octylamino, nonyl amino, decylamino, dipentylamino, dihexylamino, diheptylamino, dioctylamino, dinonylamino, didecylamino methylpentylamino, methylhexylamino, methylheptylamino, methyloctylamino, methylnonylamino, methyldecylamino, ethylpentylamino, ethylhexylamino, ethylheptylamino, ethyloctylamino, ethylnonylamino, ethyldecylamino, propylpentylamino, propylhexylamino, propylheptylamino, propyloctylamino, propylnonylamino, propyldecylamino.

The above groups may be substituted or unsubstituted, but when they are substituted, they are preferably not substituted on a carbon atom that is attached to the nitrogen atom of the amino group —NR_{b}R_{c}.

Alternatively, and although less preferred, R_{b}, R_{c} and the nitrogen atoms to which they are bound may together from a ring with between 3 and 10, preferably between 4 and 7, and most preferably 5 or 6 atoms in the ring (including the nitrogen atom to which both Rₐ and R_{b} are bound). This ring consists of one nitrogen atom, carbon atoms and optionally one further heteroatom chosen from oxygen, nitrogen and sulfur, but preferably contains only carbon atoms and 1 or 2 nitrogen atoms, most preferably only carbon atoms and only one nitrogen atom. Said ring may optionally also be substituted, and may in particular be substituted with one or more, and in particular one or two, C₁-C₆ alkyl groups; and said ring may contain a double bond and/or be aromatic (although aromatic rings may be less preferred, as they may not be easily protonated at a pH in the ranges mentioned above. For the same reason, although an amino group —NR_{b}R_{c} in which R_{b} and/or R_{c} is a substituted or unsubstituted aryl is not excluded, such amino groups are again less preferred).

Some specific, but non-limiting examples of such non-aromatic cyclic groups —NRₐR_{b} are pyrrolidinyl, piperazinyl, morpholinyl and piperidinyl, all of which may be unsubstituted and may optionally also be substituted, and may in particular be substituted with one or more, and in particular one or two, C₁-C₆ alkyl groups.

R_{d} and Rₑ may each independently be one of the groups mentioned for R_{b} and R_{c} above (including the structures in which N, R_{b} and R_{c} together form a ring), but may also each independently be substituted or unsubstituted aryl (In this respect, it should be noted that the requirement mentioned above for the amino group —NR_{b}R_{c}—i.e. that it is in essentially protonated form at a pH in the range of 5.0 and 9.0, preferably 6.0-8.0, e.g. about 7.0—may, but does not necessarily need to, apply to the amino group —NR_{d}Rₑ).

One of R_{b} and R_{c} may, together with the nitrogen atom of the amino group —NR_{b}R_{c}, one of R₁ and R₂ and the carbon atom to which R₁ and R₂ are bound, form a substituted or unsubstituted 5-, 6-, 7- or 8-membered ring that contains carbon atoms, the nitrogen atom of the amino group —NR_{b}R_{c} and optionally one further heteroatom chosen from oxygen, sulfur and nitrogen and that may be saturated or contain one double bond. Some preferred, but non-limiting examples of such groups (in which the ring is formed by R₂ and R_{c}) are:

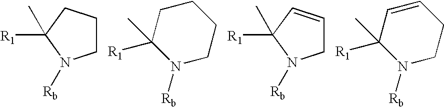

which may be substituted or unsubstituted as indicated above and in which R₁ and R_{b} are as indicated above.

Ring (1), Ring (3) and the group [C(R₁)(R₂)]ₙ—N(R_{b})R_{c}) are preferably chosen such, and connected to each other in such a way, that the distance between the at least one hydrogen-accepting heteroatom in Ring (1) and the nitrogen atom of the amino group in the group [C(R₁)(R₂)]ₙ—N(R_{b})R_{c}), as determined using a Scatter Plot (generated as indicated above), is in the range of 11.0 to 11.8, preferably from 11.0 to 11.6, more preferably from 11.0 to 11.4 Angstrom.

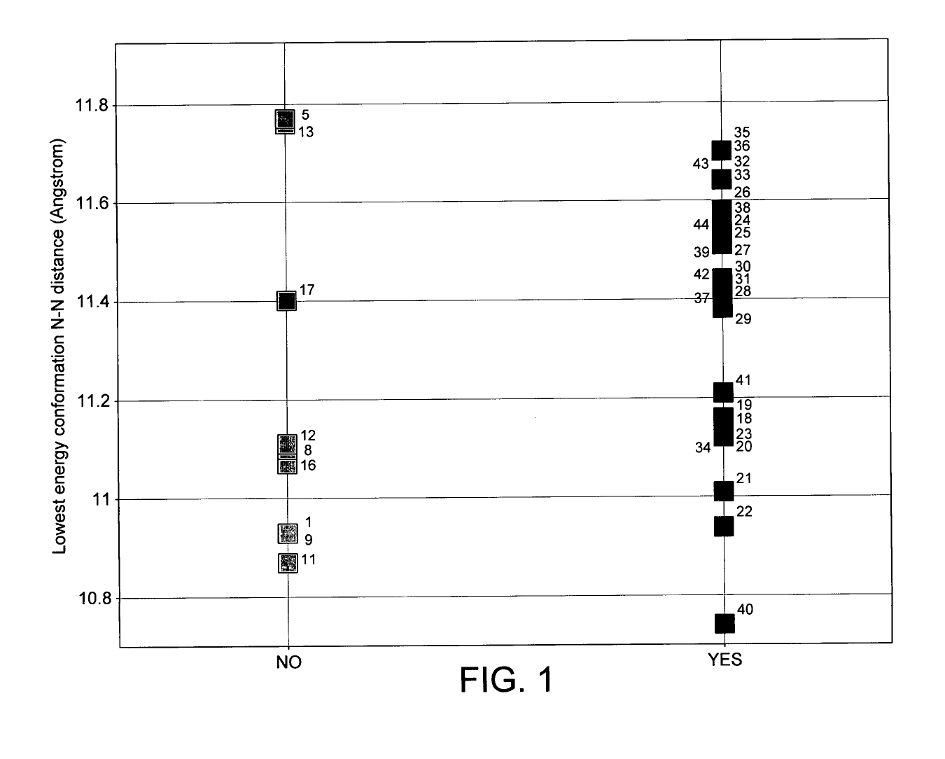

The distance between the at least one hydrogen-accepting heteroatom in Ring (1) and the nitrogen atom of the amino group in the group [C(R₁)(R₂)]ₙ—N(R_{b})R_{c}) can be determined using a commercially available computer algorithm, such as the software package MOE (Chemical Computing Group, Inc, Quebec, Canada), version 2003.02, on SGI Fuel hardware, running IRIX 6.5. Generally, the default parameters for the software can be used, unless indicated differently. In particular, this N—N distance can be calculated according to the following procedure:
The molecules are drawn using the molecule builder of MOE 2003.02. The primary amine function is protonated by forcing a positive charge on the nitrogen. Where possible, the amide function is put in a CIS position to mimic the active conformation. Molecules are minimized using the MMFF94 force field as implemented in MOE 2003.02. The default minimization parameters and procedures of MOE 2003.02 are applied.
A stochastic conformational search is applied on the minimized structure. The default parameters are applied with the exception of the option to rotate around amide and double bonds. Furthermore, the energy cutoff parameter is set to 5 kcal/mol.
The N—N distance of the energetically lowest conformation is measured using the standard procedures available in MOE 2003.02. These distances can also be represented schematically as a Scatter Plot, as shown in the FIG. 1.

According to one particularly preferred, but non-limiting embodiment, in order to achieve such a distance between the at least one hydrogen-accepting heteroatom in Ring (1) and the nitrogen atom of the amino group in the group [C(R₁)(R₂)]ₙ—N(R_{b})R_{c}), Ring (1) is a saturated, unsaturated and/or aromatic 6-membered ring with the at least one hydrogen-accepting heteroatom in the 4-position relative to the amide group —N(Rₐ)—C(═O)—, that may be fused with one or two other rings as mentioned above (i.e. Ring (6) and/or (7)); Ring (3) is a saturated, unsaturated and/or aromatic 6-membered ring in which the group [C(R₁)(R₂)]ₙ—N(R_{b})R_{c}) is in the 4-position relative to the —N(Rₐ)—C(═O)—; and the group [C(R₁)(R₂)]ₙ—N(R_{b})R_{c}) is a methyleneamino group —CR₁R₂—NR_{b}R_{c} (i.e. with n being 1 and R₁, R₂, R_{b} and R_{c} being as defined hereinabove).

However, generally, any combination of groups that is chosen for Ring (1), Ring (3) and the group [C(R₁)(R₂)]ₙ—N(R_{b})R_{c}) within the definitions mentioned above so as to achieve such a distance between the at least one hydrogen-accepting heteroatom in Ring (1) and the nitrogen atom of the amino group in the group [C(R₁)(R₂)]ₙ—N(R_{b})R_{c}) can be used in the invention.

Thus, preferably, the invention relates to compounds of general Formula I, in which:
Ring (1) is a substituted or unsubstituted, saturated, unsaturated or aromatic 4-,
5-, 6-, 7- or 8-membered ring containing carbon atoms and at least one hydrogen-accepting heteroatom and optionally 1 or 2 further heteroatoms;
Rₐ is as defined above;
Ring (3) is a substituted or unsubstituted, saturated, unsaturated or aromatic 4-,
5-, 6-, 7- or 8-membered ring containing carbon atoms optionally 1 or 2 heteroatoms;
R₁, R₂, n, R_{b} and R_{c} are as defined above and in which the amino group is such that, at a pH of between 5.0 and 9.0, preferably between 6.0 and 8.0, such as about 7.0, it is essentially in a protonated form;
   and in which
the distance between the at least one hydrogen-accepting heteroatom in Ring (1) and the nitrogen atom of the group [C(R₁)(R₂)]ₙ—N(R_{b})R_{c}), as determined using a Scatter Plot (generated as indicated above), is in the range of 11.0 to 11.8, preferably 11.0 to 11.6, and more preferably 11.0 to 11.4 Angstrom. Preferred definitions for Ring (1), Rₐ, Ring (3) and the substitutents X are as mentioned above.

According to one preferred, but non-limiting embodiment, the invention relates to a compound of the formula (II):

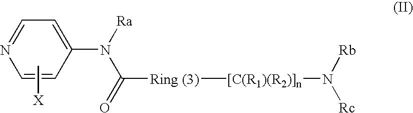

in which the 4-pyridinyl group (H):

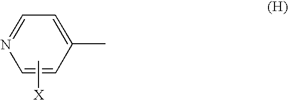

may be unsubstituted (i.e. X=hydrogen) or may be substituted with 1-4, preferably 1 or 2, substitutents X that are independently chosen from halogen, C₁-C₆ alkyl, C₁-C₆ alkoxy, substituted or unsubstituted aryl, nitro, hydroxy and an amino group NR_{d}Rₑ (in which R_{d} and Rₑ are as defined herein);

Rₐ, Ring (3) and [C(R₁)(R₂)]ₙ—N(R_{b})R_{c}) are as defined above; and the distance between the nitrogen atom in the 4-pyridinyl group (H) and the nitrogen atom of the amino group in the group [C(R₁)(R₂)]ₙ—N(R_{b})R_{c}), as determined using a Scatter Plot (generated as indicated above), is in the range of 11.0 to 11.8, preferably 11.0 to 11.6, and more preferably 11.0 to 11.4 Angstrom.

Preferred definitions for Rₐ, Ring (3) and the substitutents X are as mentioned above; and n and the groups R₁, R₂, R_{b} and R_{c} are preferably in accordance with the preferences mentioned above for the group [C(R₁)(R₂)]ₙ—N(R_{b})R_{c}).

According to one particularly preferred, but non-limiting, aspect of this preferred embodiment, the invention relates to a compound of the formula (III): 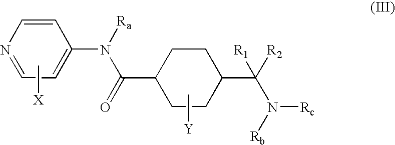
in which the 4-pyridinyl group is (H) as hereinbefore defined;
the 1,4-cyclohexylene group (J):

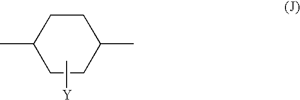

may be unsubstituted (i.e. Y=hydrogen) or may be substituted with 1-4, preferably 1 or 2, substitutents Y that are independently chosen from halogen, C₁-C₆ alkyl, C₁-C₆ alkoxy, substituted or unsubstituted aryl, nitro, hydroxy and an amino group NR_{d}Rₑ (in which R_{d} and Rₑ are as defined herein); and

Rₐ, R₁, R₂, R_{b} and R_{c} are as defined above.

Preferred definitions for Rₐ, the substitutents X, the substitutents Y, the groups R₁, R₂, Rₐ and R_{b} and n are as mentioned above.

According to another particularly preferred, but non-limiting, aspect of this preferred embodiment, the invention relates to a compound of the formula (IV): 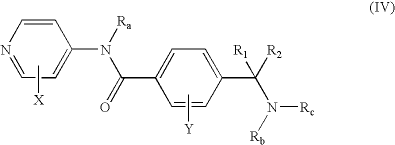
in which the 4-pyridinyl group is (H) as hereinbefore defined;
the 1,4-phenylene group (L):

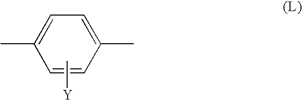

may be unsubstituted (i.e. Y=hydrogen) or may be substituted with 1-4, preferably 1 or 2, substitutents Y that are independently chosen from halogen, C₁-C₆ alkyl, C₁-C₆ alkoxy, substituted or unsubstituted aryl, nitro, hydroxy and an amino group NR_{d}Rₑ (in which R_{d} and Rₑ are as defined herein); and

Rₐ, R₁, R₂, R_{b} and R_{c} are as defined above.

Preferred definitions for Rₐ, the substitutents X, the substitutents Y, the groups R₁, R₂, Rₐ and R_{b} and n are as mentioned above.

In the case where Ring (3) is a 1,4-phenylene group, one of R₁ and R₂, the carbon atom to which R₁ and R₂ are bound, Y and two of the carbon atoms belonging to the aromatic ring to which Y is bound, may form a substituted or unsubstituted 5-, 6-, 7- or 8-membered ring that contains carbon atoms, the nitrogen atom of the amino group —NR_{b}R_{c} and optionally one further heteroatom chosen from oxygen, sulfur and nitrogen and that may be saturated or contain one double bond.

Furthermore, in the case where Ring (3) is a 1,4-phenylene group, one of R_{b} or R_{c}, the nitrogen atom to which R_{b} or R_{c} are bound, the carbon atom to which R₁ or R₂ are bound, Y and two of the carbon atoms belonging to the aromatic ring to which Y is bound, may form a substituted or unsubstituted 5-, 6-, 7- or 8-membered ring that contains carbon atoms, the nitrogen atom of the amino group —NR_{b}R_{c} and optionally one further heteroatom chosen from oxygen, sulfur and nitrogen and that may be saturated or contain one double bond.

According to another preferred, but non-limiting embodiment, the invention relates to a compound of the formula (V): 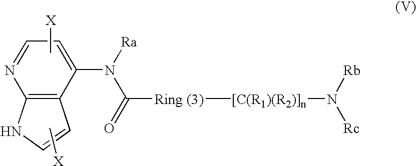
in which, in the 7-azaindole group (A):

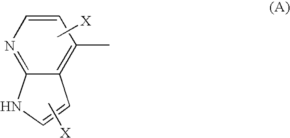

each ring may be unsubstituted (i.e. X=hydrogen) or each ring or both rings may independently be substituted with 1 or 2 substitutents X that are independently chosen from halogen, C₁-C₆ alkyl, C₁-C₆ alkoxy, substituted or unsubstituted aryl, nitro, hydroxy, a substituted or unsubstituted amino group and an amino group NR_{d}Rₑ (in which R_{d} and Rₑ are as defined herein);

Rₐ, Ring (3) and [C(R₁)R₂)]ₙ—N(R_{b})(R_{c}) are as defined above; and

the distance between the pyridine-nitrogen atom (i.e. the nitrogen atom shown in the 6-membered Ring (1) in group (A)) and the nitrogen atom of the amino group in the group [C(R₁)R₂)]ₙ—N(R_{b})(R_{c}), as determined using a Scatter Plot (generated as indicated above), is in the range of 11.0 to 11.8, preferably 11.0 to 11.6, and more preferably 11.0 to 11.4 Angstrom.

Preferred definitions for Rₐ, Ring (3) and the substitutents X are as mentioned above; and n and the groups R₁, R₂, R_{b} and R_{c} in the group [C(R₁)R₂)]ₙ—N(R_{b})(R_{c}) are preferably in accordance with the preferences mentioned above for the group [C(R₁)R₂)]ₙ—N(R_{b})(R_{c}).

According to one particularly preferred, but non-limiting, aspect of this preferred embodiment, the invention relates to a compound of the formula (VI):

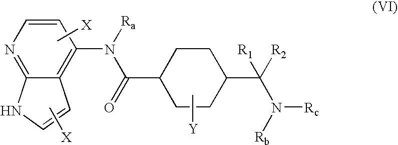

in which the 7-azaindole group is (A) as hereinbefore defined;

the 1,4-cyclohexylene group is (M) as hereinbefore defined; and

Rₐ, R₁, R₂, R_{b} and R_{c} are as defined above.

Preferred definitions for Rₐ, the substitutents X, the substitutents Y, the groups R₁, R₂, R_{b} and R_{b} and n are as mentioned above.

According to another particularly preferred, but non-limiting, aspect of this preferred embodiment, the invention relates to a compound of the formula (VII):

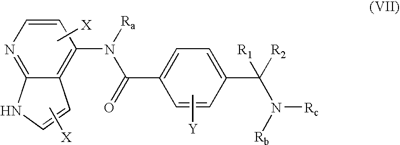

in which 7-azaindole group is (A) as hereinbefore defined;

the 1,4-phenylene group is (L) as hereinbefore defined; and

Rₐ, R₁, R₂, R_{b} and R_{c} are as defined above.

Preferred definitions for Rₐ, the substitutents X, the substitutents Y, the groups R₁, R₂, R_{b} and R_{c} and n are as mentioned above.

According to another preferred, but non-limiting embodiment, the invention relates to a compound of the formula (VIII): 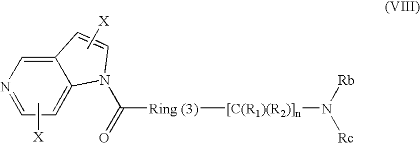
in which, in the 5-azaindole group (D):

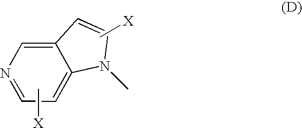

each ring may be unsubstituted (i.e. X=hydrogen) or in which each ring or both rings may independently be substituted with 1 or 2 substitutents X that are independently chosen from halogen, C₁-C₆ alkyl, C₁-C₆ alkoxy, substituted or unsubstituted aryl, nitro, hydroxy and an amino group NR_{d}Rₑ (in which R_{d} and Rₑ are as defined herein);

Ring (3) and [C(R₁)(R₂)]ₙ—N(R_{b})R_{c}) are as defined above; and in which:

the distance between the pyridine-nitrogen atom (i.e. the nitrogen atom shown in the 6-membered Ring (1) in group (D)) and the nitrogen atom of the amino group in the group [C(R₁)(R₂)]ₙ—N(R_{b})R_{c}), as determined using a Scatter Plot (generated as indicated above), is in the range of 11.0 to 11.8, preferably 11.0 to 11.6, and more preferably 11.0 to 11.4 Angstrom.

Preferred definitions for Ring (3) and the substitutents X are as mentioned above; and n and the groups R₁, R₂, R_{b} and R_{c} in the group [C(R₁)(R₂)]ₙ—N(R_{b})R_{c}) are preferably in accordance with the preferences mentioned above for the group [C(R₁)(R₂)]ₙ—N(R_{b})R_{c}).

According to one particularly preferred, but non-limiting, aspect of this preferred embodiment, the invention relates to a compound of the formula (IX):

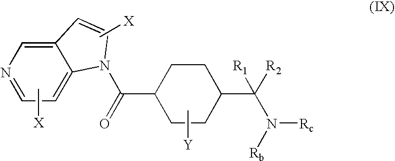

in which

the 5-azaindole group is (D) as hereinbefore defined;

the 1,4-cyclohexylene group is (J) as hereinbefore defined; and

R₁, R₂, R_{b} and R_{c} are as defined above.

Preferred definitions for the substitutents X, the substitutents Y, the groups R₁, R₂, R_{b} and R_{c} and n are as mentioned above.

According to another particularly preferred, but non-limiting, aspect of this preferred embodiment, the invention relates to a compound of the formula (X):

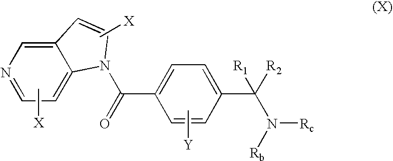

in which

the 5-azaindole group is (D) as hereinbefore defined;

the 1,4-phenylene group is (L) as hereinbefore defined; and

R₁, R₂, R_{b} and R_{c} are as defined above.

Preferred definitions for the substitutents X, the substitutents Y, the groups R₁, R₂, R_{b} and R_{c} and n are as mentioned above.

According to another preferred, but non-limiting embodiment, the invention relates to a compound of the formula (XI):

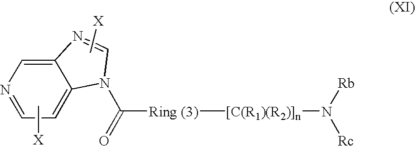

in which, in the 1H-imidazo[4,5-c]pyridine group (E):

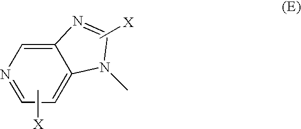

each ring may be unsubstituted (i.e. X=hydrogen) or each ring or both rings may independently be substituted with 1 or 2 substitutents X (and in the case of Ring (7) with only one such substitutent X) that are independently chosen from halogen, C₁-C₆ alkyl, C₁-C₆ alkoxy, substituted or unsubstituted aryl, nitro, hydroxy and an amino group NR_{d}Rₑ (in which R_{d} and Rₑ are as defined herein);

Ring (3) and [C(R₁)(R₂)]ₙ—N(R_{b})(R_{c}) are as defined above;

and in which:

the distance between the pyridine-nitrogen atom (i.e. the nitrogen atom shown in the 6-membered Ring (1) in group (E)) and the nitrogen atom of the amino group in the group [C(R₁)(R₂)]ₙ—N(R_{b})(R_{c}), as determined using a Scatter Plot (generated as indicated above), is in the range of 11.0 to 11.8, preferably 11.0 to 11.6, and more preferably 11.0 to 11.4 Angstrom.

Preferred definitions for Ring (3) and the substitutents X are as mentioned above; and n and the groups R₁, R₂, R_{b} and R_{c} in the group [C(R₁)(R₂)]ₙ—N(R_{b})(R_{c}) are preferably in accordance with the preferences mentioned above for the group [C(R₁)(R₂)]ₙ—N(R_{b})(R_{c}).

According to one particularly preferred, but non-limiting, aspect of this preferred embodiment, the invention relates to a compound of the formula (XII):

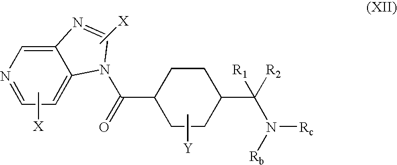

in which the 1H-imidazo[4,5-c]pyridine group is (E) as hereinbefore defined;

the group 1,4-cyclohexylene group is (J) as hereinbefore defined; and

R₁, R₂, R_{b} and R_{c} are as defined above.

Preferred definitions for the substitutents X, the substitutents Y, the groups R₁, R₂, R_{b} and R_{c} and n are as mentioned above.

According to another particularly preferred, but non-limiting, aspect of this preferred embodiment, the invention relates to a compound of the formula (XIII):

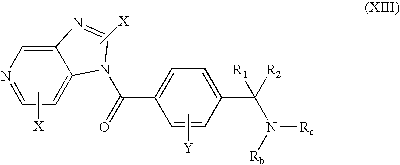

in which the group 1H-imidazo[4,5-c]pyridine group is (E) as hereinbefore defined;

the 1,4-phenylene group is (L) as hereinbefore defined; and

R₁, R₂, R_{b} and R_{c} are as defined above.

Preferred definitions for the substitutents X, the substitutents Y, the groups R₁, R₂, R_{b} and R_{c} and n are as mentioned above.

In the present description, unless indicated otherwise:

Halogen refers to fluorine, chlorine, bromine and iodine;

C₁-C₁₀ alkyl includes all linear, branched or cyclic alkyl groups with between 1 and 10 carbon atoms, and thus includes methyl, ethyl, n-propyl, i-propyl, butyl and its isomers (e.g. n-butyl, i-butyl and t-butyl); pentyl and its isomers, hexyl and its isomers, heptyl and its isomers, octyl and its isomers, nonyl and its isomers; decyl and its isomers; and cycloalkyl groups such as cyclopentyl, cyclohexyl, cycloheptyl cyclooctyl, cyclononyl and cyclodecyl (which may be further substituted with one or more alkyl groups such as methyl, ethyl, etc., as long as the total number of carbon atoms is 10 or less); and groups like cyclopentylmethylene and cyclohexylmethylene;

C₁-C₆ alkyl includes all linear, branched or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopentyl, 2-, 3- or 4-methylcyclopentyl, cyclopentylmethylene, and cyclohexyl.

C₁-C₁₀ alkoxy refers to a group —OR_{c}, in which R_{c} is C₁-C₁₀alkyl (as defined above).

C₁-C₆ alkoxy refers to a group —OR_{d} in which R_{d} is C₁-C₆alkyl. (as defined above).

Aryl refers to a substituted or unsubstituted aromatic 5-, 6-, 7- or 8-membered ring containing carbon atoms and optionally 2 or 1 heteroatoms chosen from oxygen, sulfur and nitrogen. Aryl is preferably a 5- or 6-membered ring. Aryl preferably contains only one heteroatom chosen from oxygen, sulfur and nitrogen. The heteroatom is preferably nitrogen. More preferably, aryl is a substituted or unsubstituted 5-membered ring containing carbon atoms and 2, and preferably 1 heteroatom(s), which is most preferably nitrogen; or a substituted or unsubstituted 6-membered aromatic ring containing carbon atoms and 1 and preferably no heteroatoms (i.e. phenyl). The group aryl may also be fused with another substituted or unsubstituted, saturated, unsaturated or preferably aromatic 5-, 6-, 7- or 8-membered, and preferably 5- or 6-membered, ring. Examples of suitable groups aryl will be clear to the skilled person. Most preferably, aryl is substituted or unsubstituted phenyl.

when a group is said to be “substituted”, said group may be substituted with once or more, and preferably once or twice, with substitutents chosen from halogen, hydroxy, nitro, cyano, C₁-C₆ alkyl and/or C₁-C₆ alkoxy.

Also, generally, when a carbon atom in a compound of the invention is substituted, it is preferably substituted in such a way that it is bound to only one heteroatom (i.e. other than carbon or hydrogen), it being understood that according to this preferred aspect, carbon atoms that are part of a ring, and in particular of an aromatic ring, may be bound both to a heteroatom that is part of a substitutent as well as a heteroatom that is part of the (aromatic) ring.

The compounds of the invention may be in the form of pharmaceutically and/or veterinary acceptable salts, as generally described below. Particular mention is made of compounds of the Formulae I-XIII above in which a mono-, di or tri-acid addition salt is formed between:
the at least one hydrogen-accepting heteroatom in Ring (1) and a pharmaceutically acceptable acid; and/or
the amino group —NR_{b}R_{c} and a pharmaceutically acceptable acid; and/or
any further hydrogen-accepting nitrogen atoms as may be present in Ring (1), Ring (6) or Ring (7);

or any two of these, and preferably all three of these. Some preferred, but non-limiting examples of suitable pharmaceutically acceptable organic and/or inorganic acids are as hydrochloric acid, hydrobromic acid, sulfuric acid, nitric acid, acetic acid and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a Ring (6) with a hydrogen-donating heteroatom, the invention also covers salts and/or isomers formed by transfer of said hydrogen atom to a basic group or atom within the molecule.

Also, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of the Formulae I-XIII above.

The invention also generally covers all pharmaceutically acceptable predrugs and prodrugs of the compounds of the Formulae I-XIII above, for which general reference is made to the prior art cited hereinbelow.

Some of the compounds of the invention may contain one or more asymmetric carbon atoms that serve as a chiral center, which may lead to different optical forms (e.g. enantiomers or diastereoisomers). The invention comprises all such optical forms in all possible configurations, as well as mixtures thereof.

More generally, from the above, it will be clear to the skilled person that the compounds of the invention may exist in the form of different isomers and/or tautomers, including but not limited to geometrical isomers, conformational isomers, E/Z-isomers, stereochemical isomers (i.e. enantiomers and diastereoisomers) and isomers that correspond to the presence of the same substitutents on different positions of the rings present in the compounds of the invention. All such possible isomers, tautomers and mixtures thereof are included within the scope of the invention, as long as the distance between the at least one hydrogen-accepting heteroatom in Ring (1) and the nitrogen atom of the group [C(R₁)(R₂)]ₙ—N(R_{b})(R_{c}) is within the ranges mentioned above.

Some particularly preferred compounds of the invention are the compounds of Examples 10, 12, 14, 18, 23, 24 and 25, with the compounds of Examples 10, 17, 23, 24 and 25 being particularly preferred.

The compounds of the Formulae I-XIII above may be prepared in a manner known per se for the preparation of analogous compounds, such as the methods described for the preparation of pyridinocarboxamides in U.S. Pat. No. 4,997,834 and EP 0 370 498.

The compounds of the above Formulae I-XIII may be prepared in a manner analogous to methods known per se.

One preferred, but non-limiting method comprises condensation of an amine of formula (XIV):

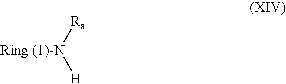

in which Ring (1) and Rₐ have the meanings indicated hereinabove, with a carboxylic acid of formula (XV):

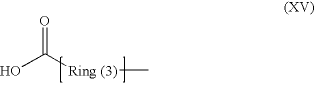

in which Ring (3) and [C(R₁)(R₂)]ₙ—N(R_{b})(R_{c}) have the meaning indicated above.

The reaction can generally be performed by coupling the compound of Formula XIV with a compound of Formula XV. In this reaction, the compound of Formula XV will usually be used as an activated acid derivative thereof, for example as an acyl halide that is obtained by converting the compound of Formula XV into an acyl chloride with thionyl chloride or oxalyl chloride using method known per se. The above reaction can be performed at a suitable molar ratio, for example of between 1:5 and 5:1, preferably between 1:1 and 1:1.5, and most preferably about 1:1; in a suitable solvent or solvent mixture, such as dichloromethane (DCM) or pyridine, at a suitable temperature, usually between 0° C. and the boiling point of the solvent used, such as between room temperature (20° C.) and 60° C. (depending on the solvent used), and for a suitable period of time, usually between 1 hr and 24 hrs, such as about 1-8 hrs, and in the presence of a suitable base (not in case of pyridine), for example an organic base such as diisopropylethylamine (DIEA), triethylamine (TEA), triisopropylamine, in an amount between 0.1 to 5.0 equivalents.

Alternative conditions for carrying out the above condensation include the use of a coupling agent, such as TBTU, HOBt, or EDCI at a suitable molar ratio, for example between 1; 1.0 to 1:3 (relative to the acid derivative); in a suitable solvent or solvent mixture, such as DCM or DMF, at a suitable temperature, usually between 0° C. and the boiling point of the solvent used, such as between RT (room temperature; 20° C.) and 60° C. (depending on the solvent used), and for a suitable period of time, usually between 1 hr and 24 hrs, such as about 1-12 hrs, and in the presence of a suitable base, for example an organic base such as DIEA0), TEA, triisopropylamine, in an amount between 0.1 to 5.0 equivalents.

Other suitable reagents and conditions for performing the above reaction between the amine of Formula XIV and the acid XV (or a suitably activated derivative thereof) will be clear to the skilled person; reference is made to the standard handbooks, such as J. March, Advanced Organic Chemistry, 3rd Edition, 1985.

The starting compounds for this reaction are either commercially available or can be prepared in a manner known per se.

The compounds of the Formulae I-XIII above may then be isolated from the reaction mixture and may optionally be further purified, using techniques known per se, such as evaporation of the solvent, washing, trituration, recrystallization from a suitable solvent or solvent mixture, and chromatographic techniques, such as column chromatography (for example using a silica gel column) or preparative thin layer chromatography. Reference is for example made to the techniques described in the Examples below and to the techniques used in the art for the purification and isolation of analogous compounds, such as the methods for the purification and/or isolation of pyridinocarboxamides described in U.S. Pat. No. 4,997,834 and EP 0 370 498.

The compounds of the invention may be used for the inhibition of kinases in vitro or in vivo, preferably in vitro, for modulating biological pathways and/or processes in which such kinases are involved; and/or to prevent and/or treat diseases or disorders in which such kinases, pathways and/or processes are involved. For example, the compounds of the invention can be used to inhibit kinases that are involved in metabolic disease, such as JNK1, p38 kinase, GSK-3, IKKbeta (IKappaB kinase beta) and p70S6K, and in particular GSK-3 (compare WO 03/82859); and/or to modulate biological pathways and/or processes in which such kinases are involved.

The compounds of the invention may also be used to inhibit kinases that are (known to be) inhibited by analogous pyridinocarboxamides (for example ROCK); to modulate biological pathways and/or processes in which such kinases are involved; and/or to prevent and/or treat diseases and disorders associated therewith.

According to one preferred, but non-limiting embodiment, the compounds of the invention may be used to inhibit (at least one isoform of) PKC; and as such may be used for any purposes known per se for inhibitors of PKC.

According to an even more preferred embodiment, the compounds of the invention may be used to inhibit at least one isoform of PKC chosen from the group of calcium-independent, but diacylglycerol- and/or phorbol ester-sensitive isoforms of PKC, and in particular the delta, epsilon, theta and/or eta isoform of PKC, more in particular the epsilon or theta isoform of PKC; and as such may be used for any purposes known per se for inhibitors of these isoforms.

According to a particularly preferred embodiment, the compounds of the invention are selective for PKC compared to other kinases. By “selective” it is meant that the compound of the invention has an IC₅₀ value for one of the PKC isoforms delta, epsilon, eta and/or theta, and in particular for PKC epsilon, that is at least 2 times smaller, preferably at least 5 times smaller, more preferably at least 10 times smaller, such as 50-100 times smaller, than the IC₅₀ value for a kinase other than one of the PKC isoforms delta, epsilon, eta and/or theta, and in particular PKC epsilon, as measured using a suitable assay and substrate for measuring the activity of a kinase, such as the assay used in the Examples below, or a similar kinase assay using a suitable substrate. For example, suitable assays and substrates for the various isoforms of PKC are described in the prior art mentioned above and/or are commercially available, such as the Protein Kinase C Assay Kits available from Invitrogen.

According to an even more particularly preferred embodiment, the compounds of the invention are selective for diacylglycerol- and/or phorbol ester-sensitive isoforms of PKC (e.g. delta, epsilon, theta and/or eta) compared to other isoforms of PKC kinases (e.g. alpha, beta-I, beta-II or gamma). By “selective” it is meant that the compound of the invention has an IC₅₀ value for one of the PKC isoforms delta, epsilon, eta and/or theta, and in particular for PKC epsilon, that is at least 2 times smaller, preferably at least 5 times smaller, more preferably at least 10 times smaller, such as 50-100 times smaller, than the IC₅₀ value for one of the other PKC isoforms, and in particular PKC gamma, as measured using a suitable assay and substrate for measuring the activity of a kinase, and in particular for an isoform of PKC, such as the assay used in the Examples below, or a similar kinase assay using a suitable substrate. For example, suitable assays and substrates for the various isoforms of PKC are described in the prior art mentioned above and/or are commercially available, such as the Protein Kinase C Assay Kits available from Invitrogen.

In the invention, particular preference is given to compounds of the Formulae I-XIII above that in the inhibition assay for PKC epsilon described below inhibit PKC epsilon with an IC₅₀ value of less than 100 μM, preferably less than 50 μM, more preferably less than 10 μM, even more preferably less than 5 μM, and in particular 1 μM or less, as determined by a suitable assay, such as the assay used in the Examples below.

More particular preference is given to compounds of the Formulae I-XIII above that in the inhibition assay for PKC epsilon described below inhibit PKC epsilon with an IC₅₀ value of less than 100 μM, preferably less than 50 μM, more preferably less than 10 μM, even more preferably less than 5 μM, and in particular 1 μM or less; and that inhibit PKC gamma with an IC₅₀ value of more than 100 μM, both as determined by a suitable assay, such as the assay used in the Examples below.

The present invention also relates to the use of the compounds of the Formulae I-XIII above in (the preparation of a composition for) inhibiting at least one kinase, in particular for inhibiting at least one isoform of PKC, more in particular for inhibiting the delta, epsilon, eta and/or theta isoform of PKC, and especially for inhibiting the epsilon and/or theta isoform of PKC. Said inhibition may be effected in vitro and/or in vivo, and when effected in vivo, is preferably effected in a selective manner, as defined above.

The compounds of the invention may generally be used for any of the pharmaceutical, veterinary applications of analogous pyridinocarboxamides known per se, such as the pharmaceutical and/or veterinary applications mentioned in U.S. Pat. No. 4,997,834 and EP 0 370 498 (e.g. those associated with ROCK).

However, according to a particularly preferred embodiment, the compounds of the invention are preferably used in the prevention and/or treatment of at least one disease or disorder in which at least one isoform of PKC is involved. Such diseases and disorders will be clear to the skilled person and are for example described in some of the prior art mentioned hereinabove.

According to an even more particularly preferred embodiment, the compounds of the invention may be used in the prevention and/or treatment of at least one disease or disorder in which the delta, epsilon, eta and/or theta isoform of PKC is involved. Such diseases and disorders will be clear to the skilled person and are for example described in some of the prior art mentioned hereinabove.

According to an especially preferred embodiment, the compounds of the invention may be used in the prevention and/or treatment of at least one disease or disorder in which the delta and/or epsilon isoform of PKC is involved. Such diseases and disorders will be clear to the skilled person and are for example described in WO 00/01895, WO 00/01415, U.S. Pat. No. 6,376,467, WO 02/102232, US 2003/0134774, WO 03/04612 and some of the further prior art mentioned hereinabove.

For example, the compounds of the invention may be used in the prevention and/or treatment of diseases and disorders such as:

metabolic diseases, such as:
(1) hyperglycemic conditions and/or other conditions and/or diseases that are (primarily) associated with (the response or sensitivity to) insulin, including but not limited to all forms of diabetes and disorders resulting from insulin resistance, such as Type I and Type II diabetes, as well as severe insulin resistance, hyperinsulinemia, and hyperlipidemia, e.g., obese subjects, and insulin-resistant diabetes, such as Mendenhall's Syndrome, Werner Syndrome, leprechaunism, lipoatrophic diabetes, and other lipoatrophies;
(2) conditions caused or usually associated with hyperglycemic conditions and/or obesity, such as hypertension, osteoporosis and/or lipodystrophy;
(3) so-called “metabolic syndrome” (also known as “Syndrome X”) which is a condition where several of the following conditions coexist: hypertension; insulin resistance; diabetes; dyslipidemia; and/or obesity;
   as well as various inherited metabolic diseases known per se; and may also be used also for preventing, treating and/or alleviating complications and/or symptoms associated with these metabolic diseases;
anxiety, addiction such as alcohol abuse or drug abuse, withdrawal syndrome, muscle spasms, convulsive seizures, epilepsy and other prophylactic and/or therapeutic uses mentioned in WO 00/01895 (for example, to modulate the action of drugs that target the GABA-A receptor);
pain, such as chronic hyperalgesia, inflammatory pain and the other diseases and disorders mentioned in WO 00/01415, U.S. Pat. No. 6,376,467, WO 02/102232, WO 03/089456 and WO 03/089457 and the further prior art listed above;
Cardiovascular disease or heart disease, as mentioned in US 2003/0134774; and also for regulating the immune system and/or regulating an immune response in a mammal, as mentioned in WO 03/04612 and/or regulating an inflammatory response in a mammal.

The compounds of the invention may also be used as an alternative for the peptide inhibitors described in WO 03/089456 and WO 03/089457, e.g. for the same disease indications mentioned in these references for the peptide inhibitors, such as the management of pain. In doing so, the compounds of the invention will have all the usual advantages of small molecules compared to small peptides, for example that they can conveniently be formulated for oral administration, that they are usually easier to manufacture, and that they often are more stable under storage.

In particular, the compounds and compositions of the invention may be used for preventing and/or treating diabetes, especially Type I and Type II diabetes and obesity, as well as the complications and/or symptoms associated therewith. “Diabetes” itself refers to a progressive disease of carbohydrate metabolism involving inadequate production or utilization of insulin and is characterized by hyperglycemia and glycosuria.

According to a specific, very preferred, embodiment, the compounds and compositions of the invention are particularly suited for preventing and/or treating Type II diabetes.

In another embodiment, the present invention relates to the use of the compounds of the Formulae I-XIII above in (the preparation of a composition for) the prevention and/or treatment of one or more of the diseases or disorders mentioned above.

In one specific non-limiting embodiment, the present invention relates to the use of the compounds of the Formulae I-XIII above in (the preparation of a composition for) the prevention and/or treatment of metabolic diseases such as diabetes and obesity.

In another specific non-limiting embodiment, the present invention relates to the use of the compounds of the Formulae I-XIII above in (the preparation of a composition for) the prevention, treatment and/or management of pain, including but not limited to chronic hyperalgesia and inflammatory pain.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087 and U.S. Pat. No. 6,372,733.

Generally, for pharmaceutical use, the compounds of the inventions may be formulated as a pharmaceutical preparation comprising at least one compound of the invention and at least one pharmaceutically acceptable carrier, diluent or excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is again made to for instance U.S. Pat. No. 6,372,778, U.S. Pat. No. 6,369,086, U.S. Pat. No. 6,369,087 and U.S. Pat. No. 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, disintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers.

Particular reference is made to the compositions, formulations (and carriers, excipients, diluents, etc. for use therein), routes of administration etc., which are known per se for analogous pyridinocarboxamides, such as those described in U.S. Pat. No. 4,997,834 and EP-A-0 370 498.

For the treatment of pain, the compounds of the invention may be used locally or systemically, e.g. as described for the peptide inhibitors of PKC in WO 03/089456 and 03/089457. For local administration, the compounds may advantageously be used in the form of a spray, ointment or transdermal patch or another suitable form for topical, transdermal and/or intradermal administration; and for systemic administration, the compounds of the invention may advantageously be administered orally.

The preparations may be prepared in a manner known per se, which usually involves mixing the active substance(s) to be used with the one or more pharmaceutically acceptable carriers, which necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, rectal, transdermal, subcutaneous, intravenous, intramuscular or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of the Formulae I-XIII above that, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg, more often between 0.1 and 500 mg, such as between 1 and 250 mg, for example about 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778,U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

Thus, in a further aspect, the invention relates to a composition, and in particular a composition for pharmaceutical use, that contains at least one compound of the invention (i.e. a compound that has been identified, discovered and/or developed using a nematode or method as described herein) and at least one suitable carrier (i.e. a carrier suitable for pharmaceutical use). The invention also relates to the use of a compound of the invention in the preparation of such a composition.

The compositions are of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a composition for veterinary use that contains at least one compound of the invention (i.e. a compound that has been identified, discovered and/or developed using a nematode or method as described herein) and at least one suitable carrier (i.e. a carrier suitable for veterinary use). The invention also relates to the use of a compound of the invention in the preparation of such a composition.

The invention will now be illustrated by means of the following synthetic and biological examples, which do not limited the scope of the invention in any way. Unless indicated otherwise, the purity of the compounds was confirmed by liquid chromatography/mass spectrometry (LC/MS), as follows:
HPLC system: Waters 2690 with photodiode array detector Waters 996; Column: C18; Gradient: solvent A (H₂O/formic acid 26.5 nM) 0%, to solvent B (CH₃CN/formic acid 17 nM) 80% in 3 min. Flow: 2.75 ml/min.
Mass spectrometer: Micromass Platform LC. Ionization: electrospray (polarity: negative and positive).

NMR spectra were determined on a Varian Mercury 300 MHz NMR using the indicated solvent as an internal reference. Melting points were determined on a Buechi B-540 and are non-corrected. All reagents used were either obtained commercially or were prepared in a manner known per se.

The Scatter Plot of all of the compounds of the invention and some comparative compounds within the range of 10.8 and 11.8, was determined as described above using the commercial software package MOE (Chemical Computing Group, Inc, Quebec, Canada), version 2003.02, on SGI Fuel hardware, running IRIX 6.5, at default parameters (unless indicated otherwise above). Compounds that, in the Biological Examples, have an IC₅₀ value for PKC epsilon of less than 100 μM (and thus are considered “active”) are shown on the right hand side, and compounds that have an IC₅₀ value for PKC epsilon of more than 100 μM (and thus are considered “inactive”) are shown on the left hand side. Active compounds as found in the present invention have a distance between the at least one hydrogen-accepting heteroatom in Ring (1) and the nitrogen atom of the amino group in the group [C(R₁)(R₂)]ₙ—N(R_{b})(R_{c}), of between 11 to 11.8 Angstrom, preferably 100.0 to 11.6, and more preferably 11.0 to 11.4 Angstrom.

### EXAMPLES

### Example 1

The following intermediates were used to prepare the compounds described herein.

### Intermediate 1: trans-4-(benzyloxycarbonylamino-methyl)-cyclohexanecarboxylic acid

To a solution of trans-4-methylamino-cyclohexanecarboxylic acid (1 g) in THF (0.25 M), were successively added aqueous 1M Na₂CO₃ (6 ml) and benzyl chloroformate (905 μL, 1.2 eq). The reaction mixture was stirred at RT for 2 days. The solvent was evaporated and the reaction mixture was acidified with 2M HCl (until pH 1-2). The solid was filtered off and washed with water (10 ml). The residue was purified by flash chromatography (DCM/MeOH 95/5, R_{f}=0.29), yielding a white powder (74% yield). ¹H NMR (300 MHz, DMSO-d6): 0.83 ppm (m, 2H); 1.21 ppm (m, 3H); 1.69 ppm (bd, 2H, J=13.0 Hz); 1.85 ppm (bd, 2H, J=13.0 Hz); 2.08 ppm (m, 1H); 2.82 ppm (t, 2H, J=6.0 Hz); 4.98 ppm (s, 2H); 7.32 ppm (m, 6H); 12.02 ppm (s, 1H); mp: 114.2-116.3° C.

### Intermediate 2: 4-cyano-N-pyridin-4-yl-benzamide

To a suspension of 4-cyano-benzoic acid (1 g) in DCM (0.5 M) was added oxalyl chloride (2.5 eq) and a few drops of DMF. The reaction mixture was stirred at RT for 15 min. The solvent was evaporated. The residue was dissolved in DCM (0.5 M). DIEA (1.2 eq) and 4-amino-pyridine (640 mg, 1 eq) were added. After completion of the reaction (2 hours), the solvent was removed under vacuum. The residue was purified by flash chromatography (DCM/MeOH 95/5, Rf=0.10), yielding a pale yellow powder (42% yield). ¹H NMR (300 MHz, DMSO-d6): 7.75 ppm (dd, 2H, J=1.5 Hz & 4.8 Hz); 8.06 ppm (m, 4H); 8.48 ppm (dd, 2H, J=1.5 & 4.8 Hz); 10.80 ppm (s, 1H); mp: 200.2-202.4° C.

### Intermediate 3: 3-cyano-N-pyridin-4-yl-benzamide

This compound was prepared according to the procedure of Intermediate 2, starting from 3-cyano-benzoic acid (1.03 g) and 4-amino-pyridine. The title product was purified by flash chromatography (DCM/MeOH 95/5, R_{f}=0.19), yielding a white powder (54% yield). ¹H NMR (300 MHz, DMSO-d6): 7.75 ppm (m, 3H); 8.07 ppm (dt, 1H, J=1.5 & 7.9 Hz); 8.23 ppm (dt, 1H, J=1.5 & 7.9 Hz); 8.40 ppm (dt, 1H, J=0.6 & 1.8 Hz); 8.49 ppm (dd, 2H, J=1.8 & 5.0 Hz); 10.74 ppm (s, 1H).

### Intermediate 4: 4-(benzyloxycarbonylamino-methyl)-benzoic acid

This compound was prepared according to the procedure of Intermediate 1, starting from 4-(aminomethyl)-benzoic acid. The title product was purified by recristallisation in toluene, yielding a white powder (50% yield). ¹H NMR (300 MHz, DMSO-d6): 4.30 ppm (d, 2H, J=6.1 Hz); 5.10 ppm (s, 2H); 7.20-7.50 ppm (m, 7H); 7.80-8.10 ppm (m, 3H); 12.90 ppm (s, 1H); mp: 194.0-195.0° C.

### Intermediate 5: 1H-pyrrolo[2,3-b]pyrindin-4-ylamine

To a solution of 7-azaindole (5 g, 42.3 mmol) in DCM (42 ml, 1M) cooled at 0° C., was portionwise added 3-chloroperoxybenzoic acid (70-75%, 29.1 g, 4 eq). The reaction mixture was stirred for 1 hour. The reaction mixture was diluted with DCM (42 ml). The solid was removed by filtration. The 1H-pyrrolo[2,3-b]pyridine 7-oxide, was extracted with aqueous 1M HCl (3×200 ml). The aqueous layer was evaporated, yielding the 1H-pyrrolo[2,3-b]pyridine 7-oxide, as an orange powder, which was used without further purification.

To the crude the 1H-pyrrolo[2,3-b]pyridine 7-oxide (5 g), was added POCl₃ (50 ml). The reaction mixture was stirred at 100° C. for 5 hours. The solution was cooled at 0° C. (with an ice-bath), and ice/water was carefully added (100 ml). An aqueous 6M NaOH was carefully added until pH=10. The precipitate was filtered off, washed with water and then dried, yielding the 4-chloro-1H-pyrrolo[2,3-b]pyridine as a brown powder (77% yield starting from the 7-azaindole).

To a solution of 4-chloro-1H-pyrrolo[2,3-b]pyridine (4.45 g) in DMF (0.5 M) were added sodium azide (5 eq) and ammonium chloride (5 eq). The reaction mixture was heated at 110° C. for 5 hours. The solvent was evaporated, and water (200 ml) was added. The product was extracted with EtOAc (3×200 ml). The combined organic layers were evaporated. The residue was purified by flash chromatography (Cyclohexane/EtOAc 7/3, R_{f}=0.15), yielding the 4-azido-1H-pyrrolo[2,3-b]pyridine as a beige powder (77% yield).

The 4-azido-1H-pyrrolo[2,3-b]pyridine (500 mg) was dissolved in EtOH, and Pd/C (10%) was added. The reaction mixture was stirred at RT for 4 hours, under H₂ (3 atm). Pd/C was removed by filtration and then the filtrate was evaporated, pyridine a beige powder (100% yield).

Intermediate 6:

### 1-(2-trimethylsilanyl-ethoxymethyl)-1H-pyrrolo[2,3-b]pyrindin-4-ylamine

To a solution of 4-azido-1H-pyrrolo[2,3-b]pyridine in DMF (0.8 M) cooled at 0° C. were added NaH (1.5 eq) and (2-chloromethoxy-ethyl)-trimethyl-silane (1.2 eq). The reaction mixture was stirred at RT for 5 hours. Water was then added, and the product was extracted with EtOAc. The organic layer was dried over MgSO₄, and then evaporated, yielding the 4-azido-1-(2-trimethylsilanyl-ethoxymethyl)-1H-pyrrolo[2,3-b]pyridine, which was used without further purification.

To a solution of the crude 4-azido-1-(2-trimethylsilanyl-ethoxymethyl)-1H-pyrrolo[2,3-b]pyridine in isopropanol (0.4 M) was slowly added NaBH₄ (1 eq). The reaction mixture was stirred at RT for 16 hours, and then water was added. The precipitate was filtered off, and the product in the filtrate was extracted with EtOAc. The organic layer was evaporated. The residue and the precipitate were mixed. The product was purified by flash chromatography (Cyclohexane/EtOAc 6/4, R_{f}=0.25), yielding the title compound as a white powder (75% yield). ¹H NMR (300 MHz, DMSO-d6): 0.00 ppm (s, 9H); 0.90 ppm (t, 2H, J=7.9 Hz); 3.56 ppm (t, 2H, J=7.9 Hz); 5.57 ppm (s, 2H); 6.29 ppm (m, 3H); 6.65 ppm (d, 1H, J=3.6 Hz); 7.28 ppm (d, 1H, J=3.6 Hz); 7.85 ppm (d, 1H, J=5.6 Hz); mp: 116.5-118.2° C.

### Example 2

The following compounds are synthesized as comparative compounds and were tested (see example 4) as controls to the compounds of the present invention.

### Compound 1: 6-Amino-hexanoic acid pyridine-4-ylamide dihydrochloric acid salt

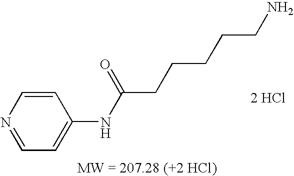

To a solution of 6-tert-butoxycarbonylamino-hexanoic acid (122.8 mg) in DMF (531 μl, 1M), were successively added DIEA (273 μl, 3 eq.) and a solution of TBTU (289 mg) and HOBt (24.3 mg) in DMF (0.5M). After stirring at RT for 3 minutes, 4-aminopyridine (50 mg, 1 eq) was added. The reaction mixture was stirred at RT for 4 hours. The solvent was evaporated and the residue was purified by flash chromatography (DCM/MeOH 9/1, R_{f}=0.60).

The resulting solid was dissolved in 3N HCl (2.7 ml). The reaction mixture was stirred at 50° C. for 3 hr. The reaction mixture was cooled down at RT. The solution was washed with DCM (5 ml). The aqueous layer was evaporated and the residue was triturated in MeOH/Pentane 2/5, yielding a white powder (70% yield). ¹H NMR (300 MHz, DMSO-d6): 1.25-1.40 ppm (m, 2H); 1.58 ppm (m, 4H); 2.45-2.55 ppm (m, 2H); 2.45-2.55 ppm (m, 2H); 2.70-2.81 ppm (m, 2H), 7.87 ppm (bs, 2H); 8.08 ppm (d, 2H, J=7.0 Hz); 8.65 ppm (d, 2H, J=7.0 Hz); 11.72 ppm (s, 1H).

### Compound 2: 4-tert-butyl-cyclohexanecarboxylic acid pyridine-4-ylamide

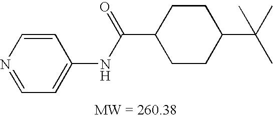

To a suspension of 4-tert-butyl-cyclohexanecarboxylic acid (74 mg) in DCM (0.5 M), was added oxalyl chloride (178 μl, 10 eq) and a few drops of DMF. The reaction mixture was stirred at RT for 1 hour. The solvent was evaporated and the residue was dissolved in DCM (0.5 M). To the solution were added pyridine (129 μl 4 eq) and 4-aminopyridine (37.7 mg, 1 eq). The reaction mixture was stirred at RT overnight. The solution washed with aqueous 1M K₂CO₃. The organic layer was evaporated. The residue was purified by flash chromatography (DCM/MeOH 95/5), yielding a white powder (60% yield). ¹H NMR (300 MHz, CDCl₃): 0.77 ppm (s, 9H); 1.01 ppm (m, 1H); 1.22 ppm (m, 2H); 1.60-1.80 ppm (m, 4H); 2.18 ppm (m, 2H); 2.67 ppm (m, 1H); 7.65 ppm (d, 2H, J=6.2 Hz); 8.07 ppm (bs, 1H); 8.38 ppm (m, 2H, J=6.2 Hz); mp: 150.0-150.8° C.

### Compound 3: Trans-4-aminomethyl-cyclohexanecarboxylic acid phenylamide hydrochloric acid salt

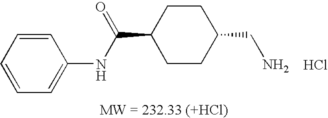

To a solution of Intermediate 1 (114 mg, 1 eq), HOBt (70 mg, 1.3 eq), EDCI.HCl (100 mg, 1.3 eq) and N-methylmorpholine (49 μl, 1.3 eq) in DMF (3 ml) was added aniline (50 μl, 1.3 eq). The reaction mixture was stirred at RT for 24 hours. The solvent was evaporated and the residue was triturated in 2M NaOH. The solid was filtered off and washed with 1M HCl, and then water. The product was purified by flash chromatography (DCM/MeOH 99.5/0.5), yielding the trans-(4-phenylcarbamoyl-cyclohexylmethyl)-carbamic acid benzyl ester as a white powder (63% yield).

To a suspension of the solid (91 mg) in MeOH (10 ml) were added Pd (10% on charcoal, 20 mg) and ammonium formate (63 mg, 4 eq). The reaction mixture was stirred at RT overnight. Ammonium formate (1 eq) was added and the reaction mixture was stirred for 24 hours. Pd was removed by filtration, then the solvent was evaporated. The residue was purified by C-18 chromatography. The compound was converted into hydrochloric acid salt (by dissolution in 1M HCl and lyophilisation), yielding a white powder (77% yield). ¹H NMR (300 MHz, DMSO-d6): 0.96 ppm (m, 2H); 1.38 ppm (m, 2H); 1.55 ppm (m, 1H); 1.83 ppm (d, 4H, J=10.8 Hz); 2.28 ppm (t, 1H, J=12.1 Hz); 2.64 ppm (t, 2H, J=5.1 Hz); 6.98 ppm (t, 1H, J=7.3 Hz); 7.24 ppm (t, 2H, J=7.3 Hz); 7.58 ppm (d, 2H, J=7.8 Hz); 7.95 ppm (bs, amine); 9.91 (s, 1H); mp: 247-249° C.

### Compound 4: Trans-4-aminomethyl-cyclohexanecarboxylic acid (4-fluoro-phenyl)-amide hydrochloric acid salt

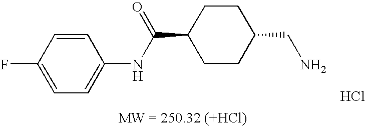

The trans-[4-(4-fluoro-phenylcarbamoyl)-cyclohexylmethyl]-carbamic acid benzyl ester was obtained in a similar manner as described for Compound 3, using Intermediate 1 and 4-fluoro-aniline, yielding a white powder (69% yield).

The title compound was obtained in a similar manner as described for Compound 3. A white powder was obtained, after conversion into its hydrochloric acid salt (41% yield). ¹H NMR (300 MHz, DMSO-d6): 0.86 ppm (m, 2H); 1.18 ppm (m, 1H); 1.36 ppm (m, 2H); 1.79 ppm (d, 4H, J=11.7 Hz); 2.21 ppm (t, 1H, J=11.7 Hz); 2.37 ppm (d, 1H, J=6.1 Hz); 2.77 ppm (t, 1H, J=6.1 Hz); 7.08 ppm (dd, 2H, J=8.7 Hz); 7.58 ppm (dd, 2H, J=8.7 Hz); 9.85 (s, 1H).

mp: 157-159° C.

### Compound 5: Trans-4-aminomethyl-cyclohexanecarboxylic acid (pyridine-4-ylmethyl)-amide

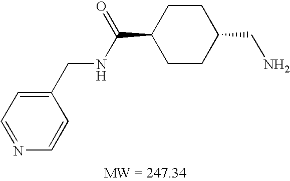

The trans-{4-[(pyridine-4-ylmethyl)-carbamoyl]-cyclohexylmethyl}-carbamic acid benzyl ester was obtained in a similar manner as described for Compound 3, using Intermediate 1 and 4-picolylamine. The product was purified by prep-HPLC, yielding a white powder (53% yield).

To a solution of trans-{4-[(pyridine-4-ylmethyl)-carbamoyl]-cyclohexylmethyl}-carbamic acid benzyl ester (55 mg) in MeOH (0.1 M) were added Pd/C (6 mg) and ammonium formate (36 mg, 4 eq). The reaction mixture was stirred at RT for 4 hours, and then filtered off through a celite cake. The celite washed with MeOH. The solvent was evaporated, yielding a pale yellow powder (86% yield). ¹H NMR (300 MHz, DMSO-d6): 0.87 ppm (m, 2H); 1.20-1.40 ppm (m, 3H); 1.78 ppm (m, 4H); 2.10 ppm (m, 1H); 2.41 ppm (d, 2H, J=6.5 Hz); 4.24 ppm (d, 2H, J=6.2 Hz); 7.18 ppm (d, 2H, J=6.2 Hz); 8.35 (bs, 1H); 8.45 ppm (bd, 2H, J=6.2 Hz).

### Compound 6: Trans-4-aminomethyl-cyclohexanecarboxylic acid pyridin-3-ylamide

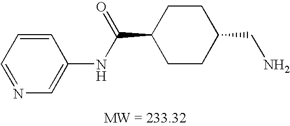

The trans-[4-(pyridine-3-ylcarbamoyl)-cyclohexylmethyl]-carbamic acid benzyl ester was obtained in a similar manner as described for Compound 3, using Intermediate 1 and 3-aminopyridine. The product was purified by prep-HPLC, yielding a white powder (25% yield).

The title product was obtained in a similar manner as described for Compound 5, yielding a beige powder (10% yield). ¹H NMR (300 MHz, DMSO-d6): 0.92 ppm (m, 2H); 1.35-1.45 ppm (m, 3H); 1.82 ppm (m, 4H); 2.30 ppm (m, 1H); 2.58 ppm (d, 2H, J=6.7 Hz); 7.29 ppm (m, 1H); 8.02 ppm (d, 2H, J=7.8 Hz); 8.20 ppm (d, 2H, J=4.0 Hz); 8.41 ppm (s, 1H); 10.14 ppm (s, 1H).

### Compound 7: Trans-4-aminomethyl-cyclohexanecarboxylic acid pyridin-2-ylamide

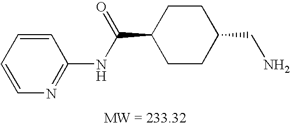

The trans-[4-(pyridine-2-ylcarbamoyl)-cyclohexylmethyl]-carbamic acid benzyl ester was obtained in a similar manner as described for Compound 3, using Intermediate 1 and 2-aminopyridine. The product was purified by prep-HPLC, yielding a white powder (15% yield).

The title product was obtained in a similar manner as described for Compound 5, yielding a beige powder (10% yield). ¹H NMR (300 MHz, DMSO-d6): 0.90 ppm (m, 2H); 1.30-1.40 ppm (m, 3H); 1.70-1.80 ppm (m, 3H); 2.30-2.35 ppm (m, 2H); 2.85-2.95 ppm (m, 2H); 7.04 ppm (m, 1H); 7.72 ppm (m, 1H); 8.05 ppm (d, 1H, J=8.2 Hz); 8.26 ppm (m, 1H); 10.33 ppm (s, 1H).

### Compound 8: 4-(1-hydroxy-ethyl)-N-pyridin-4-yl-benzamide

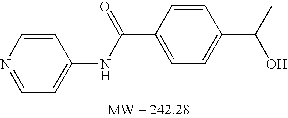

To a solution of 4-acetyl-N-pyridin-4-yl-benzamide (157 mg), in water/THF (12 ml/2 ml), was added NaBH4 (265 mg, 11 eq). The reaction mixture was stirred at RT for 6 hours. The reaction mixture was acidified by 3M HCl. The solution washed with DCM (2×10 ml). The aqueous phase was neutralized, and then evaporated, and the residue was purified by flash chromatography, yielding a white powder (64% yield). ¹H NMR (300 MHz, DMSO-d6): 1.32 ppm (d, 3H, J=6.6 Hz); 4.79 ppm (m, 1H); 5.33 ppm (d, 1H, J=4.2 Hz); 7.49 ppm (d, 2H, J=8.2 Hz); 7.76 ppm (d, 2H, J=6.0 Hz); 7.90 ppm (d, 2H, J=8.2 Hz); 8.45 ppm (d, 2H, J=6.0 Hz); 10.52 ppm (s, 1H).

### Compound 9: 3-aminomethyl-N-pyridin-4-yl-benzamide dihydrochloric acid salt

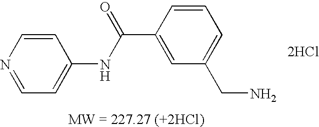

To a solution of Intermediate 3 (50 mg) in MeOH (2 ml), was added cobalt (II) chloride hexahydrate (1.2 eq) and MeOH (2 ml). NaBH₄ (3×8 eq) were then added (in 4 hours). The blue solution turned into a black suspension. The reaction mixture was stirred at RT for 4 hours. The reaction mixture was filtered through celite. The celite cake washed with MeOH. The solvent was evaporated and the product was partitioned between DCM and water. The organic phase washed with water, then concentrated. The residue was purified by C-18 chromatography, yielding a white solid (22% yield, after conversion into its dihydrochloric acid salt). ¹H NMR (300 MHz, DMSO-d6): 4.19 ppm (m, 2H); 7.64 ppm (m, 1H); 7.81 ppm (m, 1H); 78.08 ppm (d, 2H, J=7.1 Hz); 8.35 ppm (m, 2H); 8.40 ppm (m, 3H); 8.75 ppm (d, 2H, J=6.2 Hz); 9.09 ppm (bs, 1H); 11.72 ppm (s, 1H).

### Compound 10: 4-aminomethyl-N-pyridin-4-yl-benzenesulfonamide

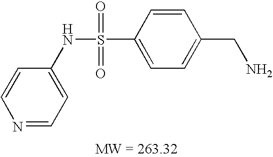

To a solution of 4-aminopyridine (100 mg) in DMF (0.2 M) were added DIEA (1.1 eq) and a solution of 4-cyano-benzenesulfonyl chloride (47 mg, 1 eq) in THF (0.25 M). The reaction mixture was stirred at RT for 4 hours. The solvent was evaporated and the residue was purified by prep-HPLC.

The 4-cyano-N-pyridin-4-yl-benzenesulfonamide (60 mg) was dissolved in THF (0.22 M). A 1M solution of BH3 in THF (5 eq) was carefully added. The reaction mixture was stirred at 30° C. for 0.5 hour. 3N HCl (3.6 eq) was then added and the reaction mixture was refluxed for 0.5 hour. The reaction mixture was cooled down at 0° C. and NaOH was added (7.2 eq). The solution was saturated with potassium carbonate and extracted with DCM. The compound was not detected in the organic phase. The aqueous layer was evaporated and the residue was purified by flash chromatography (DCM/MeOH/NH₃ sat. 90/10 to 75/25), yielding a yellow powder (32% yield). ¹H NMR (300 MHz, DMSO-d6): 3.69 ppm (bs, 2H); 6.57 ppm (d, 2H, J=6.1 Hz); 7.28 ppm (m, 2H); 7.51 ppm (d, 1H, J=8.2 Hz); 7.61 ppm (d, 2H, J=8.2 Hz); 7.79 ppm (d, 2H, J=6.1 Hz).

### Compound 11: (4-aminomethyl-phenyl)-pyridin-4-ylmethyl-amine dihydrochloric acid salt

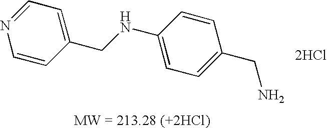

To a mixture of pyridine-4-carbaldehyde (44 mg, 1 eq) and (4-amino-benzyl)-carbamic acid tert-butyl ester (1.1 eq) in DCM (0.3 M) at 0° C., was added NaBH(Oac)₃ (130 mg). The reaction mixture was stirred at RT overnight. The solvent was evaporated, and the residue purified by prep-HPLC, yielding {4-[(pyridine-4-ylmethyl)-amino]-benzyl}-carbamic acid tert-butyl ester as a white powder (57% yield). 1H NMR (300 MHz, DMSO-d6): 1.34 ppm (s, 9H); 3.90 ppm (d, 2H, J=6.2 Hz); 4.27 ppm (d, 2H, J=5.9 Hz); 6.28 ppm (t, 1H, J=6.2 Hz); 6.44 ppm (d, 2H, J=8.5 Hz); 6.89 ppm (d, 2H, J=8.5 Hz); 7.14 ppm (t, 1H, J=5.9 Hz); 7.29 ppm (dd, 2H, J=4.4 and 1.5 Hz); 8.44 ppm (dd, 2H, J=4.4 and 1.5 Hz).

The product was dissolved in 3M HCl. The solution was heated at 80° C. for 2 hours. The solvent was evaporated, yielding the title product as a white solid (100% yield). 1H NMR (300 MHz, DMSO-d6): 3.74 ppm (d, 2H, J=5.6 Hz); 4.60 ppm (s, 2H); 6.54 ppm (d, 2H, J=8.5 Hz); 7.18 ppm (d, 2H, J=8.5 Hz); 7.95 ppm (d, 2H, J=6.8 Hz); 8.41 ppm (bs, 2H); 8.84 ppm (d, 2H, J=8.5 Hz).

### Compound 12: 4-(N-pyridin-4-yl)-benzamide oxime

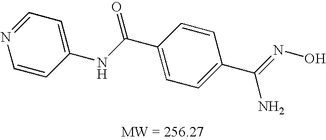

To a suspension of Intermediate 2 (48 mg), in EtOH (0.5 M), were added NH₂OH.HCl (1.5 eq) and DIEA (1.6 eq). The reaction mixture was refluxed for 2.5 hours. The reaction mixture was cooled down at RT. The solvent was evaporated. The product was triturated with water, filtered off and washed with water. The product was dried, yielding a pale yellow powder (90% yield). ¹H NMR (300 MHz, DMSO-d6): 6.24 ppm (bs, 2H); 7.85 ppm (d, 2H, J=8.2 Hz); 8.03 ppm (m, 4H); 8.58 ppm (d, 2H, J=6.4 Hz); 10.02 ppm (bs, 1H); 11.12 ppm (s, 1H); mp: 233.5-235.8° C.

### Compound 13: 4-(3-pyridin-4-yl-[1,2,4]oxadiazol-5-yl-benzylamine dihydrochloric acid salt

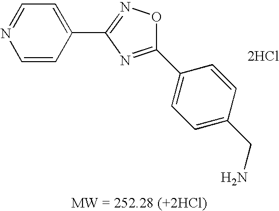

To a solution of 4-(Boc-aminomethyl)-benzoic acid (187 mg) in DMF (0.25 M) were added DIEA (5 eq), TBTU (1 eq) and HOBt (0.2 eq). The solution was stirred at RT for 3 minutes, and then isonicotinamide oxime (102 mg, 1 eq) was added. After 1 hour, the solvent was evaporated. The residue was triturated with 0.05 M NaOH (5 ml). The solid was filtered off, washed with water and dried under vacuum.

The solid was dissolved in DMF (0.25 M). The reaction mixture was heated at 110° C. for 2 hours. The reaction mixture was cooled down at RT. The precipitate was filtered off, washed with water, and then dried under vacuum.

The solid was dissolved in 3N HCl. The solution was heated at 50° C. for 2 hours. The solvent was evaporated and the residue was dried under vacuum. The title product was obtained as a white powder (74% yield). ¹H NMR (300 MHz, DMSO-d6): 4.15 ppm (q, 2H, J=5.7 Hz); 5.00 ppm (bs, 2H); 7.80 ppm (d, 2H, J=8.4 Hz); 8.24 ppm (m, 4H); 8.95 ppm (d, 2H, J=6.0 Hz).

### Compound 14: 4-(3H-imidazo[4,5-c]pyridine-2-yl)-benzylamine

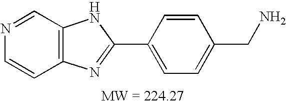

A solution of 3,4-Diaminopyridine (200 mg) and 4-cyanobenzonitrile (240 mg; 1 eq) in DMF (18.3 ml; 0.1M) was heated at 100° C. for 48 hours. The reaction mixture was cooled down at RT. The precipitate was filtered off, washed with DMF and water. The 4-(3H-imidazo[4,5-c]pyridine-2-yl)-benzonitrile was purified by flash chromatography (DCM/MeOH 98/2), yielding a white powder (64% yield).

To a solution of 4-(3H-imidazo[4,5-c]pyridine-2-yl)-benzonitrile (100 mg), in methanol (2.5 ml), was added cobalt (II) chloride hexahydrate (26.3 mg; 2.4 eq). The reaction mixture was cooled at 0° C. and NaBH₄ (209 mg, 12 eq) was added portionwise. After stirring overnight at RT, cobalt (II) chloride hexahydrate (26.3 mg; 2.4 eq) and NaBH₄ (209 mg, 12 eq) were added and the reaction stirred at RT for 4 hours. The medium was then filtered through a celite cake and the filtrate was evaporated under vacuum. The crude solid was dissolved in DCM and the organic layer extracted 3 times with water. The aqueous layers were combined and evaporated under vacuum. The residue was purified by flash chromatography (DCM/MeOH/TEA 90/9/1), yielding a white powder (63% yield). ¹H NMR (300 MHz, DMSO-d6): 4.05 ppm (m, 2H); 7.58 ppm (d, 2H, J=5.6 Hz); 7.65 ppm (d, 2H, J=7.7 Hz); 8.25 ppm (d, 2H, J=7.7 Hz); 8.29 ppm (d, 2H, J=5.6 Hz); 8.92 ppm (s, 1H).

### Compound 15: 5-(amino-methyl)-furan-2-carboxylic acid pyridine-4-ylamide dihydrochloric acid salt

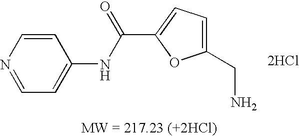

To a solution of 4-amino-pyridine (1 eq) in pyridine (0.25M) was added the furan-2-carbonyl chloride (814 μl, 8.23 mmol) dissolved in a minimum of DCM. The reaction mixture was stirred at 50° C. for 2 hours, and then evaporated. The residue was taken in saturated aqueous NaHCO₃ and then extracted with DCM. The combined organic layers were evaporated. The furan-2-carboxylic acid pyridine-4-ylamide was purified by flash chromatography (DCM/MeOH 95/5, R_{f}=0.28), yielding a white powder (68% yield). ¹H NMR (300 MHz, DMSO-d6): 6.72 ppm (m, 1H); 7.40 ppm (d, 1H, J=3.2 Hz); 7.75 ppm (d, 2H, J=6.5 Hz); 7.97 ppm (s, 1H); 8.44 ppm (d, 2H, J=6.5 Hz); 10.53 ppm (s, 1H). mp: 159.0-159.9° C.

To a solution of the amide (100 mg) in concentrated sulfuric acid (0.5 ml) was added N-hydroxymethylphtalimide (188 mg, 2 eq). The reaction mixture was stirred at RT for 3 hours, and then diluted with EtOH and water. The reaction mixture was evaporated. The resulting oil was diluted in an aqueous solution of saturated NaHCO₃ (pH=8). The product was extracted with DCM (3×20 ml). The combined organic layers were evaporated. The residue was purified by flash chromatography (DCM/MeOH(NH₃ sat.) 99/1 to 95/5), yielding the 5-(1,3-dioxo-1,3-dihydro-isoindol-2-ylmethyl)-furan-2-carboxylic acid pyridine-4-ylamide (85% purity).

To a solution of the crude 5-(1,3-dioxo-1,3-dihydro-isoindol-2-ylmethyl)-furan-2-carboxylic acid pyridine-4-ylamide (198 mg) in EtOH (10 ml), was added hydrazine hydrate (2 ml). The reaction mixture was stirred at 50° C. for 1 hour. The solution was evaporated. The resulting solid was dissolved in water, and the product was extracted with DCM (3×20 ml). The combined organic layers were evaporated. The residue was purified by flash chromatography (DCM/MeOH(NH₃ sat.) 99/1 to 96/4). The recovered product was dissolved in 6N HCl. The solution was evaporated and the product was dried overnight, yielding the title product as a white powder (45% yield). ¹H NMR (300 MHz, DMSO-d6): 3.53 ppm (bs, NH₂); 4.22 ppm (d, 2H, J=5.0 Hz); 6.81 ppm (d, 1H, J=3.6 Hz); 7.83 ppm (d, 1H, J=3.6 Hz); 8.52 ppm (d, 2H, J=7.2 Hz); 8.75 ppm (d, 2H, J=7.2 Hz); 12.03 ppm (s, 1H).

mp: >265° C.

### Compound 16: 4-(acetylamino-methyl)-N-pyridin-4-ylamide

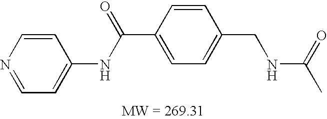

To a solution of 4-(aminomethyl)-benzoic acid (100 mg), in THF (1.3 ml, 0.5 M), was added acetic anhydride (620 μl, 10 eq). The reaction mixture was stirred at RT for 4 hours. The solvent was removed under reduced pressure. The residue was triturated with 1M HCl and then, the reaction mixture was acidified by 3M HCl. The 4-(acetylaminomethyl)benzoic acid was collected by filtration, washed with water and dried. The product was obtained as a white powder (57% yield).

To a solution of 4-(acetylamino-methyl)benzoic acid (73 mg, 1 eq), HOBt (67 mg, 1.3 eq), EDCI.HCl (94 mg, 1.3 eq) and N-methylmorpholine (47 μl, 1.3 eq) in DMF (1.5 ml) was added 4-amino-pyridine (1 eq). The reaction mixture was stirred at RT for 72 hours. The solvent was evaporated and the resulting oil was diluted with DCM. The solution washed with aqueous 1M Na₂CO₃. The organic layer was evaporated. The residue was purified by flash chromatography (DCM/2M N₃ in MeOH 95/5), yielding the title product as a white powder (10% yield). ¹H NMR (300 MHz, DMSO-d6): 1.88 ppm (s, 3H); 4.31 ppm (d, 2H, J=6.0 Hz); 7.39 ppm (d, 2H, J=8.4 Hz); 7.76 ppm (dd, 2H, J=1.5 & 4.8 Hz); 7.90 ppm (d, 1H, J=8.4 Hz); 8.45 ppm (d, 2H, J=4.8 Hz); 10.51 ppm (s, 1H).

### Compound 17: 4-(1-dimethylamino-methyl)-N-pyridin-4-ylamide

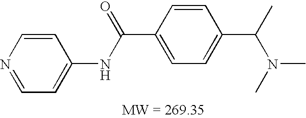

To a solution of Compound 24 (32 mg) in water (2 mL) were added DIEA (34.1 μl), formic acid (1.2 ml) and paraformaldehyde (20 eq.). The reaction mixture was stirred at RT for 10 days, and was evaporated. The residue was taken in 1M NaHCO₃ and the product was extracted with DCM. The title product was then extracted with 6M HCl and lyophilized, yielding a white powder (93% yield). ¹H NMR (300 MHz, DMSO-d6): 1.66 ppm (d, 3H, J=6.9 Hz); 2.50-2.57 ppm (m, 3H); 2.71-2.78 ppm (m, 3H); 4.52-4.64 ppm (m, 1H); 7.83 ppm (d, 2H, J=8.3 Hz); 8.15 ppm (d, 2H, J=8.3 Hz); 8.36 ppm (d, 2H, J=7.1 Hz); 8.75 ppm (d, 2H, J=7.1 Hz); 11.76 ppm (s, 1H).

### Example 3

The following compounds were synthesized and are active compounds according to the present invention. These compounds were tested in example 4.

### Compound 18: the-(+)-trans-N-(4-pyridyl)-4-(1-aminoethyl)-cyclohexanecarboxamide dihychloric acid salt

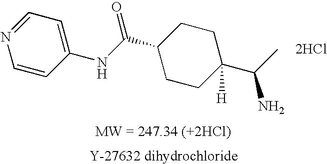

This compounds was obtained from CALBIOCHEM (Compound Y 27632, Cat. No. 688000).

### Compound 19: Trans-4-aminomethyl-cyclohexanecarboxylic acid pyridin-4-ylamide

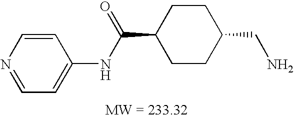

The trans-[pyridinedin-4-ylcarbamoyl)-cyclohexylmethyl]-carbamic acid benzyl ester was obtained in a similar manner as described for Compound 3, using Intermediate 1 and 4-aminopyridine, yielding after purification by flash chromatography (DCM/MeOH 97/3), a white powder (15% yield).

The title compound was obtained in a similar manner as described for Compound 3. A white powder was obtained (50% yield). ¹H NMR (300 MHz, DMSO-d6): 0.88-0.96 ppm (m, 2H); 1.20-1.46 ppm (m, 3H); 1.83 ppm (m, 3H); 2.30 ppm (m, 1H); 2.58 ppm (d, 2H, J=6.7 Hz); 2.94 ppm (d, 1H, J=7.5 Hz); 7.55 ppm (d, 2H, J=5.7 Hz); 8.36 ppm (d, 2H, J=5.7 Hz); 8.40 (bs, 2H); 10.37 (s, 1H).

### Compound 20: 4-aminomethyl-N-pyridin-4-yl-benzamide dihydrochloric acid salt

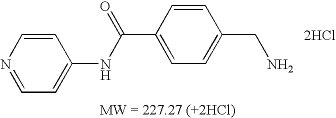

The title product was obtained in a similar manner as described for Compound 8, starting from Intermediate 2, yielding a white powder after conversion into its dihydrochloric acid salt (24% yield). ¹H NMR (300 MHz, DMSO-d6): 4.13 ppm (bd, 2H); 7.67 ppm (d, 2H, J=8.5 Hz); 8.10 ppm (d, 2H, J=8.5 Hz); 8.33 ppm (d, 2H, J=6.7 Hz); 8.45 ppm (bs, 3H); 8.73 ppm (d, 2H, J=6.7 Hz). 11.65 ppm (s, 1H).

### Compound 21: 4-aminomethyl-N-pyrimidin-4-yl-benzamide dihydrochloric acid salt

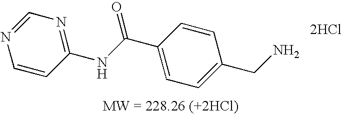

A solution of Intermediate 4 (50 mg) in thionyl chloride (1 ml) was heated at 40° C. for 2.5 hours. The solution was cooled down to RT, and then evaporated under vacuum. The resulting solid was dissolved in DCM (0.2 ml) and the solution was added dropwise to a solution of 4-amino-pyrimidine (16.7 mg, 1 eq) in pyridine (1 ml, 0.17 M). After stirring at 100° C. for 1 hour, the reaction mixture was cooled down to RT, and then evaporated under vacuum. The resulting solid was dissolved in DCM. The organic layer washed with 1M K₂CO₃, with water, dried over MgSO₄ and evaporated under vacuum, yielding the [4-(pyrimidin-4-ylcarbamoyl)-benzyl]-carbamic acid benzyl ester as an orange powder (43% yield).

To a solution of [4-(pyrimidin-4-ylcarbamoyl)-benzyl]-carbamic acid benzyl ester (27 mg), in methanol (5 ml), was added ammonium formate (37.6 mg, 8 eq) and Pd/C-10% (5 mg). After 6 hours stirring at RT, the mixture was filtered through a celite cake and the filtrate evaporated under vacuum. The residue was dissolved in HCl 1N, the aqueous layer washed with DCM, and then evaporated under vacuum, yielding a beige powder (27% yield). ¹H NMR (300 MHz, DMSO-d6): 4.20 ppm (m, 2H); 7.68 ppm (d, 2H, J=8.2 Hz); 8.15 ppm (d, 2H, J=8.2 Hz); 8.30 ppm (d, 2H, J=5.3 Hz); 8.42 ppm (m, 3H); 8.82 ppm (m, 1H); 9.06 ppm (s, 1H); 11.4 ppm (s, 1H).

### Compound 22: 5-(1-amino-ethyl)-thiophene-2-carboxylic acid pyridinedin-4-ylamide

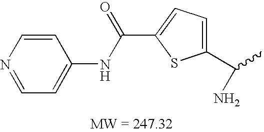

To a suspension of 5-acetyl-thiophene-2-carboxylic acid (840 mg, 4.93 mmol) in DCM (0.25M) was added oxalyl chloride (2.5 eq) and a few drops of DMF. The reaction mixture was stirred at RT for 2 hours, and then evaporated, yielding the 5-acetyl-thiophene-2-carbonyl chloride.

To a solution of 4-amino-pyridine (1 eq) in pyridine (0.25M) was added the 5-acetyl-thiophene-2-carbonyl chloride dissolved in a minimum of DCM. The reaction mixture was stirred at 50° C. for 2 hours, and then evaporated. The residue was taken in saturated aqueous NaHCO₃ and then extracted with DCM. The combined organic layers were evaporated. The 5-acetyl-thiophen-2-carboxylic acid pyridinedin-4-ylamide was purified by flash chromatography (DCM/MeOH 95/5, R_{f}=0.11), yielding a pale pink powder (40% yield). ¹H NMR (300 MHz, DMSO-d6): 2.57 ppm (s, 3H); 7.72 ppm (dd, 2H, J=9.7 & 1.5 Hz); 8.00 ppm (d, 1H, J=4.1 Hz); 8.08 ppm (d, 1H, J=4.1 Hz); 8.48 ppm (dd, 2H, J=9.7 & 1.5 Hz); 10.72 ppm (bs, 1H).

To a solution of 5-acetyl-thiophen-2-carboxylic acid pyridinedin-4-ylamide (398 mg), in absolute EtOH (7 ml, 0.25M), were added DIEA (450 μl, 1.6 eq) and hydroxylamine, HCl (180 mg, 1.6 eq). The reaction mixture was refluxed for 6 hours. The reaction mixture was cooled down at RT, and then concentrated. Water was added and the solid was collected by filtration, yielding the 5-(1-hydroxyimino-ethyl)-thiophene-2-carboxylic acid pyridinedin-4-ylamide as a white powder (87% yield).

To a solution of 5-(1-hydroxyimino-ethyl)-thiophene-2-carboxylic acid pyridinedin-4-ylamide (367 mg) in acetic acid (5 ml), was added activated zinc (551 mg, 6 eq). The reaction mixture was stirred at RT for 4 hours. Zinc was removed by filtration, and the solvent was evaporated. The residue was taken in aqueous 2M NaOH, and the product was extracted with DCM (3*10 ml). The combined organic layers were evaporated. The residue was purified by flash chromatography (DCM/2M N₃ in MeOH 90/10), yielding the title product as a beige powder (37% yield). ¹H NMR (300 MHz, DMSO-d6): 1.33 ppm (d, 3H, J=6.6 Hz); 2.18 ppm (bs, 2H); 4.22 ppm (q, 1H, J=6.6 Hz); 7.03 ppm (dd, 1H, J=1.6 & 3.9 Hz); 7.71 ppm (dd, 2H, J=1.6 & 6.3 Hz); 7.87 ppm (d, 1H, J=3.9 Hz); 8.44 ppm (dd, 2H, J=1.6 & 6.3 Hz); 10.39 ppm (s, 1H); mp: 122.1-123.3° C.

### Compound 23: 4-(1-amino-ethyl)-N-pyridin-4-yl-benzamide dihydrochloric acid salt

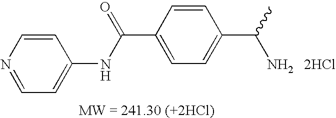

The 4-acetyl-N-pyridin-4-yl-benzamide was prepared according to the procedure of Compound 22, starting from 4-acetyl-benzoic acid (500 mg) and 4-amino-pyridine. This product was purified by flash chromatography (DCM/MeOH 95/5), yielding a pale yellow powder (92% yield).

The title product was obtained in a similar manner as described for Compound 22, starting from the 4-acetyl-N-pyridin-4-yl-benzamide (420 mg). After extraction, and evaporation of the combined organic layers, the free base of the title product was converted into its dihydrochloric acid salt, yielding a white powder (61% overall yield). ¹H NMR (300 MHz, DMSO-d6): 1.51 ppm (d, 3H, J=6.6 Hz); 4.50 ppm (t, 1H, J=6.6 Hz); 7.71 ppm (d, 2H, J=8.4 Hz); 8.13 ppm (d, 2H, J=8.4 Hz); 8.39 ppm (d, 2H, J=6.5 Hz); 8.64 ppm (m, 3H); 8.74 ppm (d, 2H, J=6.5 Hz); 11.81 ppm (s, 1H).

### Compound 24: 4-(1-amino-propyl)-N-pyridin-4-yl-benzamide dihydrochloric acid salt

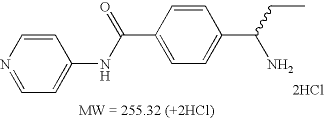

The 4-propionyl-N-pyridin-4-yl-benzamide was prepared according to the procedure of Compound 22, starting from 4-propionyl-benzoic acid (100 mg) and 4-amino-pyridine. This product was purified by flash chromatography (DCM/MeOH 99/1 to 97/3), yielding a white powder (49% yield).

The title product was obtained in a similar manner as described for Compound 22, starting from the 4-propionyl-N-pyridin-4-yl-benzamide. After extraction, and evaporation of the combined organic layers, the free base of the title product was converted into its dihydrochloric acid salt, yielding a white powder (75% overall yield). 1H NMR (300 MHz, D₂O): 0.77 ppm (t, 3H, J=7.4 Hz); 1.95 ppm (m, 2H); 4.27 ppm (dd, 1H, J=8.6 & 8.6 Hz); 7.51 ppm (d, 2H, J=8.4 Hz); 7.91 ppm (d, 2H, J=8.5 Hz); 8.15 ppm (d, 2H, J=7.4 Hz); 8.50 ppm (d, 2H, J=7.4 Hz).

### Compound 25: 4-(1-amino-3,3-dimethyl-butyl)-N-pyridin-4-yl-benzamide dihydrochloric acid salt

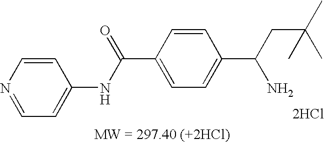

The 4-(3,3-dimethyl-butyryl)-N-pyridin-4-yl-benzamide was prepared according to the procedure of Compound 22, starting from 4-(3,3-dimethyl-butyryl)-benzoic acid (100 mg) and 4-amino-pyridine. This product was purified by flash chromatography (DCM/MeOH 99/1 to 97/3), yielding a white powder (79% yield).

The title product was obtained in a similar manner as described for Compound 22, starting from the 4-(3,3-dimethyl-butyryl)-N-pyridin-4-yl-benzamide (103 mg), yielding a white powder (90% overall yield).). ¹H NMR (300 MHz, D₂O): 0.63 ppm (s, 9H); 1.73 ppm (m, 1H); 2.04 ppm (m, 1H); 4.40 ppm (dd, 1H, J=10.3 & 3.2 Hz); 7.54 ppm (d, 2H, J=8.5 Hz); 7.83 ppm (d, 2H, J=8.5 Hz); 8.06 ppm (d, 2H, J=7.6 Hz); 8.43 ppm (d, 2H, J=7.6 Hz).

### Compound 26: 4-(1-amino-cyclopropyl-ethyl)-N-pyridin-4-yl-benzamide

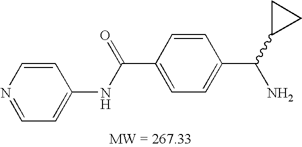

The title compound was prepared according to the procedure of Compound 22, starting from 4-cyclopropanecarbonyl-benzoic acid (160 mg) and 4-amino-pyridine, yielding a white powder (29% overall yield). ¹H NMR (300 MHz, DMSO-d6): 0.02-0.20 ppm (m, 4H); 0.70 ppm (m, 1H); 2.99 ppm (d, 1H, J=8.1 Hz); 3.06 ppm (bs, 2H); 7.30 ppm (d, 2H, J=8.3 Hz); 7.51 ppm (d, 2H, J=6.4 Hz); 7.63 ppm (d, 2H, J=8.3 Hz); 8.20 ppm (d, 2H, J=6.4 Hz); 10.25 ppm (s, 1H).

### Compound 27: 4-(1-amino-cyclopentyl-methyl)-N-pyridin-4-yl-benzamide dihydrochloric acid salt

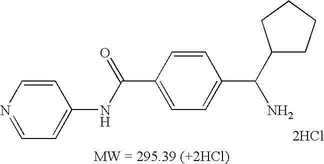

The 4-cyclopentanecarbonyl-N-pyridin-4-yl-benzamide was prepared according to the procedure of Compound 22, starting from 4-cyclopentanecarbonyl-benzoic acid (100 mg, 0.46 mmol) and 4-amino-pyridine. This product was purified by flash chromatography (DCM/MeOH 98/2 to 95/5), yielding a white powder (75% yield).

The title product was obtained in a similar manner as described for Compound 22, starting from the 4-cyclopentanecarbonyl-N-pyridin-4-yl-benzamide (93 mg). After extraction, and evaporation of the combined organic layers, the free base of the title product was converted into its dihydrochloric acid salt, yielding a white powder (61% overall yield). ¹H NMR (300 MHz, D₂O): 0.99 ppm (m, 1H); 1.21-1.55 (m, 6H); 1.83 ppm (m, 1H); 2.30 ppm (m, 1H); 4.06 ppm (d, 1H, J=10, 5 Hz); 7.44 ppm (d, 2H, J=8.4 Hz); 7.84 ppm (d, 2H, J=8.4 Hz); 8.09 ppm (d, 2H, J=7.5 Hz); 8.44 ppm (d, 2H, J=7.5 Hz).

### Compound 28: 4-(1-amino-cyclohexyl-methyl)-N-pyridin-4-yl-benzamide dihydrochloric acid salt

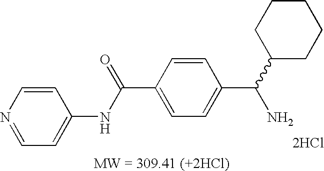

The 4-cyclohexanecarbonyl-N-pyridin-4-yl-benzamide was prepared according to the procedure of Compound 22, starting from 4-cyclohexanecarbonyl-benzoic acid (150 mg) and 4-amino-pyridine. The title product was purified by flash chromatography (DCM/MeOH 98/2 to 95/5), yielding a white powder (70% yield).

The title product was obtained in a similar manner as described for Compound 22, starting from the 4-cyclohexanecarbonyl-N-pyridin-4-yl-benzamide. After extraction, and evaporation of the combined organic layers, the free base of the title product was converted into its dihydrochloric acid salt, yielding a white powder (67% overall yield). ¹H NMR (300 MHz, DMSO-d6): 0.82-1.85 ppm (5 m, 11H); 2.12 ppm (bs, 2H); 3.60 ppm (d, 1H, J=6.9 Hz); 7.43 ppm (d, 2H, J=8.4 Hz); 7.75 ppm (d, 2H, J=1.5 Hz); 7.86 ppm (d, 2H, J=8.4 Hz); 8.45 ppm (d, 2H, J=1.5 Hz).

### Compound 29: 1,2,3,4-tetrahydro-isoquinoline-6-carboxy-pyridin-4-yl-amide dihydrochloric acid salt

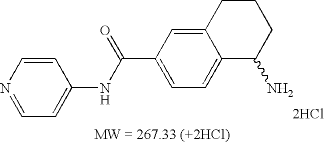

The 5-Oxo-5,6,7,8-tetrahydro-naphthalene-carboxy-pyridin-4-yl-amide was prepared according to the procedure of Compound 22, starting from 5-oxo-5,6,7,8-tetrahydro-naphthoic acid (100 mg, 0.5 mmol) and 4-amino-pyridine. This product was purified by flash chromatography (DCM/MeOH 99/1), yielding a white powder (90% yield).

The title product was obtained in a similar manner as described for Compound 22, starting from the 5-Oxo-5,6,7,8-tetrahydro-naphthalene-carboxy-pyridin-4-yl-amide. After extraction, and evaporation of the combined organic layers, the free base of the title product was converted into its dihydrochloric acid salt, yielding a white powder (60% overall yield). ¹H NMR (300 MHz, D₂O): 1.75 ppm (m, 2H); 1.87 ppm (m, 1H); 2.04 ppm (m, 1H); 2.60-2.75 ppm (m, 2H); 4.47 ppm (t, 1H, J=5.3 Hz); 7.37 ppm (d, 1H, J=9.1 Hz); 7.59 ppm (m, 2H); 8.06 ppm (dd, 2H, J=7.6 & 1.2 Hz); 8.43 ppm (dd, 2H, J=7.6 & 1.2 Hz).

### Compound 30: N-pyridin-4-yl-4-pyrrolidin-2-yl-benzamide dihydrochloric acid salt

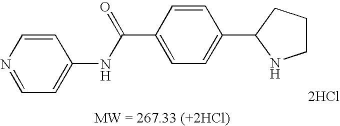

To a solution of N—BOC-2-(4-carboxy-phenyl)-pyrrolidine (100 mg), TBTU (143 mg, 1.3 eq), HOBt (14 mg, 0.3 eq) and DIEA (175 μl, 3 eq) in DMF (1.4 ml, 0.25M), was added 4-aminopyridine (32 mg, 1 eq). The reaction mixture was stirred at RT for 6 hours. The solvent was evaporated and the residue was dissolved in DCM. The organic layer washed with aqueous 1M NaHCO₃ (2×20 ml) and then was evaporated. The residue was purified by flash chromatography (DCM/MeOH 95/5). The residue was dissolved in aqueous 3M HCl. The reaction mixture was heated at 55° C. for 2 hours. The reaction mixture was cooled down at RT, and then washed with DCM (3 ml). The aqueous layer was evaporated under reduced pressure, yielding the title product as a beige powder (80% yield). ¹H NMR (300 MHz, D₂O): 2.09-2.18 ppm (m, 3H); 2.44 ppm (m, 1H); 3.40 ppm (m, 2H); 7.55 ppm (d, 2H, J=8.7 Hz); 7.90 ppm (d, 2H, J=8.7 Hz); 8.14 ppm (d, 2H, J=6.0 Hz); 8.50 ppm (d, 2H, J=6.0 Hz).

### Compound 31: 4-piperidin-2-yl-N-pyridin-4-yl-benzamide dihydrochloric acid salt

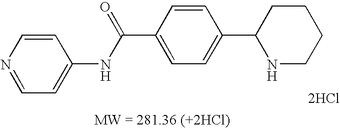

To a solution of 4-piperidin-2-yl-benzoic acid methyl ester HCl (116 mg) and triethylamine (192 μl, 3 eq) in acetonitrile was added (BOC)₂O (120 mg, 1.2 eq). The reaction mixture was stirred at RT for 1 hour. The solvent was evaporated. The residue was purified by flash chromatography (DCM/MeOH 99/1 to 98/2), yielding the N—BOC-4-piperidin-2-yl-benzoic acid methyl ester as a white powder (96% yield)

The N—BOC-4-piperidin-2-yl-benzoic acid methyl ester (140 mg) was dissolved in a mixture of MeOH (5 ml) and aqueous 1M NaOH (3 ml). The reaction mixture was heated at 60° C. for 1 hour, then cooled down at RT. MeOH was evaporated. The solution was acidified with 2M HCl. The product was extracted with DCM. The combined organic layers was evaporated. The N—BOC-4-piperidin-2-yl-benzoic acid was used for the next step without further purification.

The N—BOC-4-piperidin-2-yl-N-pyridin-4-yl-benzamide was obtained in a similar manner as described in Example 29, using N—BOC-4-piperidin-2-yl-benzoic acid and 4-aminopyridine, yielding a pale yellow powder (80% yield).

The N—BOC-4-piperidin-2-yl-N-pyridin-4-yl-benzamide was dissolved in aqueous 3M HCl (5 ml). The reaction mixture was heated at 55° C. for 2 hours. The reaction mixture was cooled down at RT, and then washed with DCM (3 ml). The aqueous layer was evaporated under reduced pressure, yielding the title product as a white powder (98% yield). ¹H NMR (300 MHz, D₂O): 1.60-2.08 ppm (m, 6H); 3.10 ppm (m, 1H); 3.45 ppm (m, 1H); 7.53 ppm (d, 2H, J=8.4 Hz); 7.90 ppm (d, 2H, J=8.4 Hz); 8.16 ppm (d, 2H, J=7.5 Hz); 8.51 ppm (d, 2H, J=7.5 Hz).

### Compound 32: 1,2,3,4-tetrahydro-isoquinoline-6-carboxylic acid pyridinedin-4-yl-amide dihydrochloric acid salt

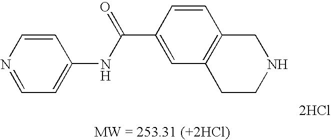

The title product was obtained in a similar manner as described for Compound 32, starting from 1,2,3,4-tetrahydro-isoquinoline-6-carboxylic acid methyl ester, yielding a white powder (72% overall yield).). ¹H NMR (300 MHz, D₂O): 3.16 ppm (t, 2H, J=6.3 Hz); 3.52 ppm (t, 3H, J=6.3 Hz); 4.42 ppm (s, 2H); 7.35 ppm (d, 1H, J=8.4 Hz); 7.75 ppm (d, 1H, J=1.8 Hz); 7.77 ppm (s, 1H); 8.18 ppm (d, 2H, J=7.5 Hz); 8.55 ppm (d, 2H, J=7.5 Hz).

### Compound 33: 4-(4,5-dihydro-1H-imidazol-2-yl)-N-pyridin-4-yl-benzamide

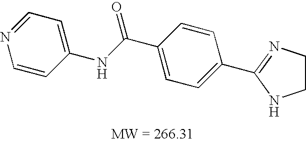

A mixture of Intermediate 2 (50 mg), 1,2-ethylenediamine (1 g, 75 eq) and P₄S₁₀ (7 mg, 0.07 eq) was heated at 90° C. for 2 hours. The reaction mixture was cooled down at RT. The 1,2-ethylenediamine in excess was evaporated under vacuum. Water (3 ml) was then added and the reaction mixture was stirred at RT until the yellow color disappeared. The precipitate was filtered off and washed with water, yielding a white powder (58% yield). ¹H NMR (300 MHz, DMSO-d6): 3.33 ppm (s, 2H); 3.62 ppm (s, 2H); 6.87 ppm (bs, 1H); 7.77 ppm (d, 2H, J=5.9 Hz); 7.95 ppm (m, 4H); 8.47 ppm (d, 2H); 10.65 ppm (s, 1H); mp>250° C.

### Compound 34: N-pyridin-4-yl-4-(1,4,5,6-tetrahydro-1H-pyrimidin-2-yl)-benzamide

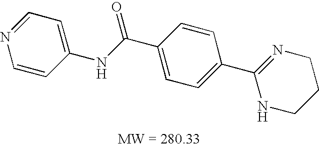

This compound was obtained in a similar manner as described for Compound 34 using Intermediate 2 and 1,3-diaminopropane, yielding a white powder (42% yield). ¹H NMR (300 MHz, DMSO-d6): 1.68 ppm (m, 2H); 3.35 ppm (4H in the signal of water); 7.77 ppm (d, 2H, J=6.0 Hz); 7.88 ppm (d, 2H, J=8.5 Hz); 7.95 ppm (d, 2H, J=8.5 Hz); 8.46 ppm (d, 2H, J=6.0 Hz); 10.62 (bs, 1H); mp: 277.6-278.7° C.

### Compound 35: 4-(1-amino-phenyl-methyl)-N-pyridin-4-yl-benzamide

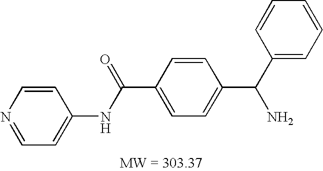

The 4-benzoyl-N-pyridin-4-yl-benzamide was prepared according to the procedure of Compound 22, starting from 4-benzoyl-benzoic acid (250 mg, 1.11 mmol) and 4-amino-pyridine. This product was purified by extraction with AcOEt, yielding a pale yellow powder (90% yield).

The title product was obtained in a similar manner as described for Compound 22, starting from the 4-benzoyl-N-pyridin-4-yl-benzamide (100 mg). The title product was purified by flash chromatography (DCM/MeOH NH₃ sat. 100/0 to 95/5), yielding a beige powder (49% overall yield). ¹H NMR (300 MHz, DMSO-d6): 5.16 ppm (s, 1H); 7.12-7.20 ppm (m, 1H); 7.23-7.31 ppm (m, 1H); 7.38-7.42 ppm (m, 2H); 7.56 ppm (d, 2H, J=8.3 Hz); 7.74 ppm (dd, 2H, J=4.8 & 1.5 Hz); 7.85 ppm (d, 2H, J=8.3 Hz); 8.44 ppm (dd, 2H, J=4.8 & 1.5 Hz); 10.48 ppm (s, 1H); mp: 76.8-77.6° C.

### Compound 36: 4-[1-amino-(4-fluorophenyl)-methyl]-N-pyridin-4-yl-benzamide dihydrochloric acid salt

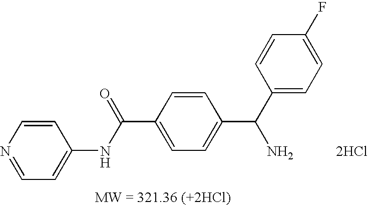

The 4-(4-fluoro-benzoyl)-N-pyridin-4-yl-benzamide was prepared according to the procedure of Compound 22, starting from 4-(4-fluoro-benzoyl)-benzoic acid (55 mg) and 4-amino-pyridine. This product was purified by flash chromatography (DCM/MeOH 95/5), yielding a white powder (57% yield).

The title product was obtained in a similar manner as described for Compound 22, starting from the 4-(4-fluoro-benzoyl)-N-pyridin-4-yl-benzamide (41 mg), yielding a pale yellow powder (27% overall yield). ¹H NMR (300 MHz, DMSO-d6): 5.82 ppm (s, 1H); 7.28 ppm (m, 2H); 7.61 ppm (m, 2H); 7.73 ppm (d, 2H, J=8.4 Hz); 8.12 ppm (d, 2H, J=8.4 Hz); 8.34 ppm (d, 2H, J=7.2 Hz); 8.73 ppm (d, 2H, J=7.2 Hz); 9.3 ppm (bs, amine); 11.74 ppm (s, 1H).

### Compound 37: 4-[1-amino-(4-methoxyphenyl)-methyl]-N-pyridin-4-yl-benzamide

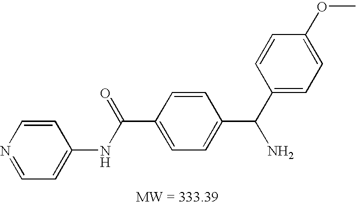

The 4-(4-methoxy-benzoyl)-N-pyridin-4-yl-benzamide was prepared according to the procedure of Compound 22, starting from 4-(4-methoxy-benzoyl)-benzoic acid (200 mg) and 4-amino-pyridine. This product was purified by flash chromatography (DCM/MeOH 95/5), yielding a white powder (83% yield).

The title product was obtained in a similar manner as described for Compound 22, starting from the 4-(4-methoxy-benzoyl)-N-pyridin-4-yl-benzamide, yielding a pale yellow powder (35% overall yield). ¹H NMR (300 MHz, DMSO-d6): 3.73 ppm (s, 3H); 5.70 ppm (s, 1H); 6.97 ppm (d, 2H, J=8.9 Hz); 7.45 ppm (d, 2H, J=8.8 Hz); 7.72 ppm (d, 2H, J=8.5 Hz); 8.13 ppm (d, 2H, J=8.5 Hz); 8.41 ppm (d, 2H, J=7.4 Hz); 8.75 ppm (d, 2H, J=7.4 Hz); 9.24 ppm (bs, amine); 11.86 ppm (s, 1H).

### Compound 38: 4-(1-amino-ethyl)-naphthalene-1-carboxylic acid pyridinedin-4-ylamide dihydrochloric acid salt

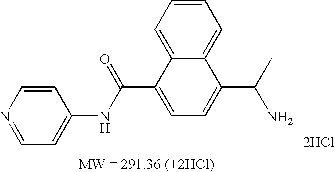

A solution of 1-Bromo-naphthalene (10 g, 48.3 mmol) and acetyl chloride (4.2 ml, 58 mmol) in 1,2-dichloroethane (100 ml) was cooled to 0° C. and aluminum chloride (14.4 g, 108 mmol) was added portion wise. The mixture was stirred at RT for 24 hours. The reaction mixture was poured into ice-water (100 ml). The two layers were separated and the water layer was extracted with diethyl ether (3×150 ml). The combined organic layers were dried over magnesium sulfate, filtered and the solvent was removed under reduced pressure to give an orange colored oil. The 1-(4-bromo-naphthalen-1-yl)-ethanone was purified by flash chromatography (cyclohexane/ethylacetate:95/5), yielding an yellow oil (91% yield).

The 1-(4-bromo-naphthalen-1-yl)-ethanone oxime was prepared according to the procedure described for Compound 22, yielding a white powder (98% yield).

Activated zinc dust (24.7 g, 379 mmol) was added portion wise to a suspension of the oxime (10.0 g, 37.9 mmol) in acetic acid (40 ml). The mixture was stirred at RT for 2 hours. The zinc dust was removed by filtration and acetic acid was removed under reduced pressure. Water (100 ml) was added and the pH was adjusted to pH=13 with 1N NaOH. The water layer was extracted with EtOAc (3×100 ml). The combined organic layers were dried over MgSO₄, filtered and the solvent was removed under reduced pressure, yielding a yellow oil (70% yield).

Boc₂O (7.1 g, 31.8 mmol) was added to a solution of the amine (6.6 g, 26.5 mmol) in 1,4-dioxane (50 ml). The reaction mixture was stirred at RT for 2 hours. The solvent was removed under reduced pressure and the product was purified by flash chromatography (cyclohexane/EtOAc:95/5), yielding a yellow powder (75% yield). The bromide (350 mg, 1 mmol) was dissolved in THF (13 ml)/water (2 ml). Potassium acetate (100 mg, 1 mmol), 1,3-bis-diphenylphosphinopropane (9.0 mg, 0.02 mmol) and palladium-(II)-acetate (9.0 mg, 0.04 mmol) were added. The mixture was stirred at 50 atm CO pressure and 150° C. for 3 hours. The reaction mixture was filtered, the filtrate dried over MgSO₄ and the solvent was removed under reduced pressure to give a yellow-greenish oil (300 mg). The 4-(1-tert-butoxycarbonylamino-ethyl)-naphthalene-1-carboxylic acid was purified by flash chromatography (DCM/MeOH:90/10), yielding a white powder (14% yield).

The title product was prepared according to the procedure of Compound 31, starting from 4-(1-tert-butoxycarbonylamino-ethyl)-naphthalene-1-carboxylic acid (44 mg) and 4-amino-pyridine (67% yield). ¹H NMR (300 MHz, DMSO-d6): 1.64 ppm (d, 3H, J=6.6 Hz); 5.3 ppm (q, 1H, J=6.5 Hz), 7.71 ppm (m, 1H), 8.00 ppm (d, 1H, J=7.7 Hz), 8.32 ppm (m, 1H), 8.35 ppm (d, 1H, J=7.3 Hz), 8.81 ppm (d, 2H, J=7.2 Hz), 12.2 ppm (s, 1H).

### Compound 39: 4-aminomethyl-2,5-dimethyl-N-pyridin-4-yl-benzamide dihydrochloric acid salt

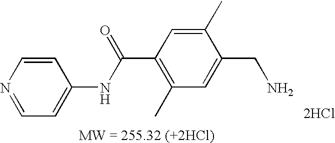

2,5-Dimethylbenzylamine (2.0 g, 14.8 mmol) was dissolved in DMF (30 ml) and phthalic anhydride (2.6 g, 17.8 mmol) was added. The mixture was stirred at 150° C. for 18 hours. The solvent was removed under reduced pressure and the residue was taken up into dichloromethane (200 ml). The organic phase was extracted with water (3×200 ml), dried and the solvent was removed. The product was purified by flash chromatography (Chloroform), yielding the 2-(2,5-dimethyl-benzyl)-isoindole-1,3-dione as an yellow powder (36%). ¹H-NMR (300 MHz, DMSO-d6): 2.16 ppm (s, 3H); 2.33 ppm (s, 3H), 4.70 ppm (s, 2H), 6.88 ppm (s, 1H), 6.95 ppm (d, 1H, J=7.3 Hz), 7.06 ppm (d, 1H, J=7.4 Hz), 7.88 ppm (m, 4H).

Aluminum chloride (1.5 g, 11.4 mmol) was added portion wise to a solution of the protected amine (1.4 g, 5.2 mmol) and acetylchloride (440 μl, 6.2 mmol) in 1,2-dichloroethane (30 ml). The mixture was stirred at 100° C. for 4 hours. The mixture was poured into ice-water (150 ml) and the water layer was extracted with chloroform (3×200 ml). The combined organic phases were dried over MgSO₄, filtered and the solvent was removed under reduced pressure. The product was purified by flash chromatography (CHCl₃/EtOAc:85/15), yielding the 2-(4-acetyl-2,5-dimethyl-benzyl)-isoindole-1,3-dione (46% yield). ¹H-NMR (300 MHz, DMSO-d6): 2.30 ppm (s, 3H); 2.40 ppm (s, 3H), 2.52 ppm (s, 3H), 4.75 ppm (s, 2H), 6.99 ppm (s, 1H), 7.66 ppm (s, 1H), 7.88 ppm (m, 4H).

2 N Sodium hydroxide (30 ml) was added to the protected amine (494.0 mg). The mixture was stirred at 150° C. for 6 hours. The reaction mixture was extracted with DCM (5×50 ml). The combined organic phases were dried over MgSO₄, filtered and the solvent was removed under reduced pressure to give a brown oil (122 mg, 43%). The 1-(4-aminomethyl-2,5-dimethyl-phenyl)-ethanone was used without any further purification in the next step.

To a solution of the amine (120 mg, 0.68 mmol) in 1,4-dioxane (10 ml), di-tert-butyldicarbonate (17 mg) was added. The mixture was stirred at room temperature for 16 hours. The solvent was removed under reduced pressure. The product was purified by flash chromatography (Pentane/EtOAc:90/10) to give a white powder (54.0 mg, 29%). Sodium hydroxide (44 mg, 1.1 mmol) was dissolved in water (3 ml) and cooled to 0° C. The ketone (38.0 mg, 0.14 mmol) dissolved in methanol (2 ml) was added, followed by NaOCl (50 ml). The mixture was stirred at room temperature for 3 hours.

The solution was neutralized to pH=7 with 1N HCl and extracted with dichloromethane (5×50 ml). The combined organic phases were dried over magnesium sulfate, filtered and the solvent was removed under reduced pressure to give a slightly yellow solid product (35 mg, 91%). The product was without any further purification used in the next step. ¹H-NMR (300 MHz, DMSO-d6): 1.39 (s, 9H), 2.23 ppm (s, 3H); 2.46 ppm (s, 3H), 4.09 ppm (d, 2H, J=5.9 Hz), 7.05 ppm (s, 1H), 7.36 ppm (t, 1H, J=5.9 Hz), 7.61 ppm (s, 1H,), 12.64 ppm (broad s, 1H).

The title product was prepared according to the procedure of Compound 32, starting from 4-(tert-butyloxycarbonylamino-methyl)-2,5-dimethyl-benzoic acid and 4-aminopyridine, yielding a white powder (44% yield). ¹H-NMR (300 MHz, DMSO-d6): 2.38 ppm (s, 3H); 2.49 ppm (s, 3H), 4.02 ppm (d, 2H, J=5.3 Hz), 7.42 ppm (s, 1H), 7.52 ppm (s, 1H,), 8.27 ppm (d, 2H, J=7.0 Hz), 8.75 (d, 2H, J=6.9 Hz), 8.61 ppm (broad s, 2H), 11.85 ppm (s, 1H).

### Compound 40: 5-(1-amino-ethyl)-thiophene-2-carboxylic acid N-(1H-pyrrolo[2,3pyridinedin-4-yl)-benzamide dihydrochloric acid salt

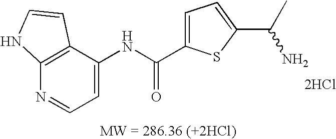

To a suspension of Intermediate 7 (180 mg) in DCM (0.25 M), were added oxalyl chloride (2.5 eq) and DMF (1 drop). The mixture was stirred at RT for 2 hours, and then evaporated, yielding the corresponding acyl chloride. The acyl chloride was dissolved in acetonitrile (1.2 ml), and then added to a solution of Intermediate 5 (35 mg) in acetonitrile (0.25 M). The reaction mixture was stirred overnight at RT under a nitrogen atmosphere. The reaction mixture was evaporated. The residue was taken in water. The resulting powder was isolated by filtration. The product was dried and used without further purification. Sodium methoxide (1 eq) was added to a solution of the previous compound (188 mg) in MeOH (3 ml). The reaction mixture was stirred at RT for 30 minutes. Water was added to the suspension, and the product was extracted with EtOAc. The residue obtained after evaporation of the organic phase was purified by preparative HPLC, yielding a beige powder (32% yield).

A solution of 5-(1-benzyloxycarbonylamino-ethyl)-thiophene-2-carboxylic acid N-(1H-pyrrolo[2,3pyridinedin-4-yl)-benzamide (36 mg) and 10% Pd/C (5 mg) in 6 ml of a mixture MeOH/3M HCl (1/1), was stirred at RT under 3 atm of hydrogen for 2 hours. The palladium was removed by filtration, and then the filtrate was evaporated, yielding a beige powder. ¹H NMR (300 MHz, DMSO-d6): 1.61 ppm (d, 3H, J=6.9 Hz); 4.90 ppm (m, 1H); 7.01 ppm (m, 1H); 7.40 ppm (d, 1H, J=3.9 Hz); 7.51 ppm (m, 1H); 7.84 ppm (d, 1H, J=6.0 Hz); 8.19 ppm (d, 1H, J=3.9 Hz); 8.28 ppm (d, 1H, J=6.0 Hz); 10.90 ppm (s, 1H); 12.40 ppm (s, 1H).

### Compound 41: 4-(1-amino-ethyl)-N-(1H-pyrrolo[2,3pyridinedin-4-yl)-benzamide dihydrochloric acid salt

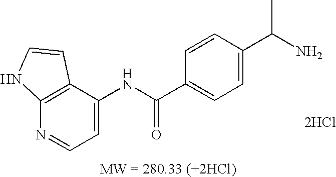

The 4-(1-benzyloxycarbonylamino-ethyl)-benzoic acid was obtained according to the procedure used for Intermediate 7 starting from 4-acetyl-benzoic acid methyl ester (51% overall yield).

The title product was prepared according to the procedure of Compound 40, starting from 4-(1-benzyloxycarbonylamino-ethyl)-benzoic acid and Intermediate 5, yielding a white powder (35% yield). ¹H NMR (300 MHz, DMSO-d6): 1.33 ppm (d, 3H, J=6.9 Hz); 4.18 ppm (q, 1H, J=6.9 Hz); 6.79 ppm (dd, 1H, J=3.5 and 1.5 Hz); 7.36 ppm (broad t, J=3.5 Hz); 7.56 ppm (d, 2H, J=8.1 Hz); 7.68 ppm (d, 1H, J=5.4 Hz); 7.94 ppm (d, 2H, J=8.1 Hz); 8.14 ppm (d, 1H, J=5.4 Hz); 10.29 ppm (s, 1H); 11.57 ppm (s, 1H).

### Compound 42: 4-(1-amino-cyclopentyl-ethyl)-N-(1H-pyrrolo[2,3pyridinedin-4-yl)-benzamide dihydrochloric acid salt

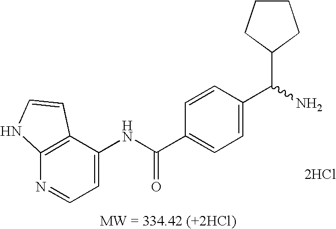

The 4-(1-benzyloxycarbonylamino-cyclopentyl-methyl)-benzoic acid was obtained according to the procedure used for Intermediate 7 starting from 4-cyclopropanecarbonyl-benzoic acid ethyl ester (47% overall yield).

The title product was prepared according to the procedure of Compound 40, starting from 4-(1-benzyloxycarbonylamino-cyclopentyl-methyl)-benzoic acid and Intermediate 5, yielding a white powder (% yield). ¹H NMR (300 MHz, DMSO-d6+D₂O): 0.98 ppm (m, 1H); 1.15-1.60 ppm (m, 6H); 1.90 ppm (m, 1H); 2.35 ppm (m, 1H); 4.15 ppm (d, 1H, J=9.9 Hz); 7.08 ppm (d, 1H, J=3.6 Hz); 7.54 ppm (d, 1H, J=3.3 Hz); 7.65 ppm (d, 2H, J=8.1 Hz); 8.02 ppm (m, 3H); 8.33 ppm (d, 1H, J=6.3 Hz).

### Compound 43: 1,2,3,4-tetrahydro-isoquinoline-6-carboxylic acid-N-(1H-pyrrolo[2,3pyridinedin-4-yl)-benzamide

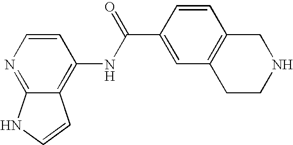

The N-Cbz-1,2,3,4-tetrahydro-isoquinoline-6-carboxylic acid was obtained according to the procedure used for Intermediate 7 starting from 1,2,3,4-tetrahydro-isoquinoline-6-carboxylic acid methyl ester (78% overall yield).

The title product was prepared according to the procedure of Compound 40, starting from N-Cbz-1,2,3,4-tetrahydro-isoquinoline-6-carboxylic acid and Intermediate 5, yielding a beige powder (35% yield). ¹H NMR (300 MHz, DMSO-d6): 3.11 ppm (t, 2H, J=5.7 Hz); 3.40-3.50 ppm (2H in the signal of water); 4.35 ppm (bs, 2H); 7.06 ppm (m, 1H); 7.41 ppm (d, 1H, J=7.8 Hz); 7.52 ppm (m, 1H); 7.85 ppm (s, 1H); 7.97 ppm (d, 1H, J=6.3 Hz); 8.31 ppm (d, 1H, J=6.3 Hz); 10.88 ppm (s, 1H); 12.37 ppm (s, 1H).

### Compound 44: 4-piperidin-2-yl-N-(1H-pyrrolo[2,3pyridinedin-4-yl)-benzamide dihydrobromic acid salt

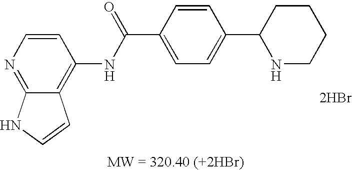

To a suspension of 4-piperidin-2-yl-benzoic acid methyl ester HCl (150 mg), DIEA (100 μl, 1 eq), and aqueous 2M Na₂CO₃ (1.17 ml) in THF was added benzyl chloroformate (92 μl, 1.1 eq). The reaction mixture was stirred at RT overnight. The solvent was evaporated. The residue was suspended in water, and extracted with DCM. The organic layer was evaporated, yielding the N-Cbz-4-piperidin-2-yl-benzoic acid methyl ester (100% yield).

To the N-Cbz-4-piperidin-2-yl-benzoic acid methyl ester, was added a solution of EtOH/aqueous 1M NaOH (5 ml/3 ml). The reaction mixture was stirred at 55° C. for 1 hour. EtOH was evaporated under reduced pressure. The solution was then acidified with 2M HCl (pH=1), and the product was extracted with DCM. The organic layer was evaporated, yielding N-Cbz-4-piperidin-2-yl-benzoic acid a white powder (100% yield). To a suspension of N-Cbz-4-piperidin-2-yl-benzoic acid (100 mg) in DCM (4 ml), was added oxalyl chloride (2.5 eq) and a few drops of DMF. The reaction mixture was stirred at RT for 2 hours, and then evaporated, yielding the N-Cbz-4-piperidin-2-yl-benzoyl chloride.

To a solution of 1-(2-trimethylsilanyl-ethoxymethyl)-1H-pyrrolo[2,3pyridinedin-4-ylamine (78 mg, 1 eq) in pyridine (4 ml) was added the N-Cbz-4-piperidin-2-yl-benzoyl chloride dissolved in a minimum of DCM. The reaction mixture was stirred at 50° C. for 2 hours, and then evaporated. The residue was taken in saturated aqueous NaHCO₃ and then extracted with DCM. The combined organic layers was evaporated. The residue was purified by flash chromatography (DCM/MeOH 99/1 to 97/3), yielding the 2-{4-[1-(2-trimethylsilanyl-ethoxymethyl)-1H-pyrrolo[2,3pyridinedin-4-ylcarbamoyl]phenyl}-piperidine-1-carboxylic acid benzyl ester as a yellow oil (40% yield).

A solution of 2-{4-[1-(2-trimethylsilanyl-ethoxymethyl)-1H-pyrrolo[2,3pyridinedin-4-ylcarbamoyl]phenyl}-piperidine-1-carboxylic acid benzyl ester in 4M HCl in dioxane was heated at 75° C. for 3 hours. The solvent was evaporated. The residue was taken in water and aqueous 1M NaOH was added (until pH˜10). The product was extracted with EtOAC (2×10 ml). The combined organic layers was evaporated, and the residue was purified by flash chromatography (DCM/MeOH 99/1 to 97/3), yielding a mixture of 2-[4-(1H-pyrrolo[2,3pyridinedin-4-ylcarbamoyl)-phenyl]-piperidine-1-carboxylic acid benzyl ester and 2-[4-(1-hydromethyl-1H-pyrrolo[2,3pyridinedin-4-ylcarbamoyl)-phenyl]-piperidine-1-carboxylic acid benzyl ester. To a solution of this mixture in MeOH/THF (0.3/0.6 ml) was added a solution of sodium acetate (40 eq) in water (0.8 ml). The reaction mixture was refluxed for 2 hours. After cooling to RT, water (3 ml) was added, and the product was extracted with DCM. The organic layer was evaporated, yielding the 2-[4-(1H-pyrrolo[2,3pyridinedin-4-ylcarbamoyl)-phenyl]-piperidine-1-carboxylic acid benzyl ester (35% yield).

A solution of 2-[4-(1H-pyrrolo[2,3pyridinedin-4-ylcarbamoyl)-phenyl]-piperidine-1-carboxylic acid benzyl ester in 30% HBr in AcOH, was heated at 40° C. for 2 hours. The reaction mixture was evaporated. The residue was dissolved in MeOH (by heating), and EtOAC was slowly added. The precipitate was filtered off, and dried, yielding the title compound, as a white powder (51% yield).). ¹H NMR (300 MHz, D₂O): 1.60 ppm (m, 2H); 1.88 ppm (m, 4H); 3.04 ppm (m, 1H); 3.38 ppm (m, 1H); 3.36 ppm (d, 1H, J=12.6 Hz); 4.20 ppm (dd, 1H, J=12.6 & 2.7 Hz); 6.64 ppm (d, 1H, J=3.6 Hz); 7.36 ppm (d, 1H, J=3.6 Hz); 7.46 ppm (d, 2H, J=8.1 Hz); 7.67 ppm (d, 1H, J=3.0 Hz); 7.86 ppm (d, 2H, J=8.1 Hz); 8.10 ppm (d, 1H, J=3.0 Hz).

### Compound 45: 4-(1-amino-cyclobutyl-ethyl)-N-pyridin-4-yl-benzamide

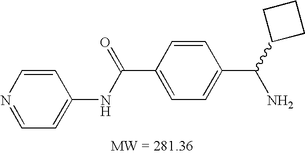

The title compound is prepared according to the procedure of Compound 22, starting from 4-cyclobutanecarbonyl-benzoic acid and 4-amino-pyridine.

### Compound 46: 4-(1-amino-2,2-dimethyl-butyl)-N-pyridin-4-yl-benzamide

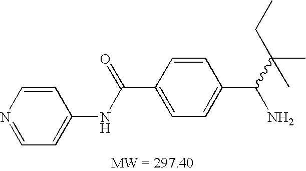

The title compound is being prepared according to the procedure of Compound 22, starting from 4-(2,2-dimethyl-butyryl)-benzoic acid and 4-amino-pyridine.

### Compound 47: 1-amino-indan-5-carboxylic acid pyridinedin-4-yl-amide

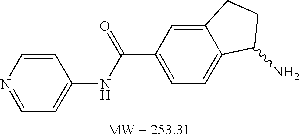

The title compound is being prepared according to the procedure of Compound 22, starting from 1-indanone-5-carboxylic acid and 4-amino-pyridine.

### Compound 48: 4-(1-amino-butyl)-N-pyridin-4-yl-benzamide

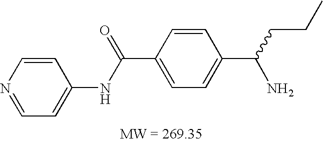

The title compound is prepared according to the procedure of Compound 22, starting from 4-butanoyl-benzoic acid and 4-amino-pyridine.

### Compound 49: 4-(1-amino-pentyl)-N-pyridin-4-yl-benzamide

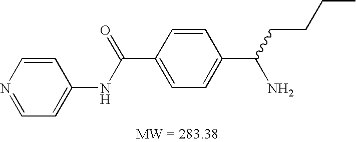

The title compound is prepared according to the procedure of Compound 22, starting from 4-pentanoyl-benzoic acid and 4-amino-pyridine.

### Compound 50: 4-(1-amino-2-methyl-propyl)-N-pyridin-4-yl-benzamide

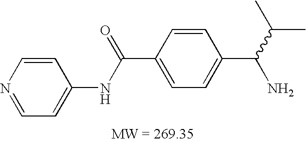

The title compound is prepared according to the procedure of Compound 22, starting from 4-isobutyryl-benzoic acid and 4-amino-pyridine.

### Compound 51: 4-(1-amino-2,2-dimethyl-propyl)-N-pyridin-4-yl-benzamide

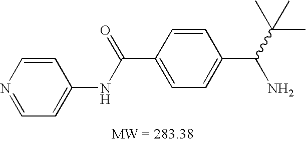

The title compound is prepared according to the procedure of Compound 22, starting from 4-(2,2-dimethyl-propionyl)-benzoic acid and 4-amino-pyridine.

### Compound 52: 4-(1-amino-propyl)-N-(1H-pyrrolo[2,3pyridinedin-4-yl)-benzamide

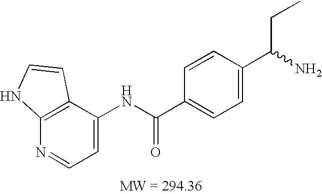

The 4-(1-benzyloxycarbonylamino-propyl)-benzoic acid was obtained according to the procedure used for Intermediate 7 starting from 4-acetyl-benzoic acid methyl ester (66% overall yield). The title product is prepared according to the procedure of Compound 41, starting from 4-(1-benzyloxycarbonylamino-propyl)-benzoic acid and Intermediate 5.

### Compound 53: 4-(1-amino-cyclopropyl-ethyl)-N-(1H-pyrrolo[2,3pyridinedin-4-yl)-benzamide

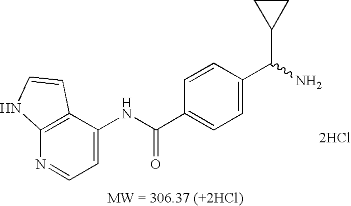

The title product is prepared according to the procedure of Compound 41, starting from 4-cyclopropanecarbonyl-benzoic acid and Intermediate 5.

### Compound 54: 4-(1-amino-cyclobutyl-ethyl)-N-(1H-pyrrolo[2,3pyridinedin-4-yl)-benzamide

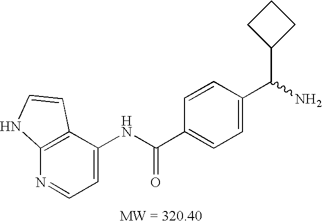

The title product is prepared according to the procedure of Compound 41, starting from 4-cyclobutanecarbonyl-benzoic acid and Intermediate 5.

### Compound 55: 4-(1-amino-2,2-dimethyl-butyl)-N-(1H-pyrrolo[2,3pyridinedin-4-yl)-benzamide

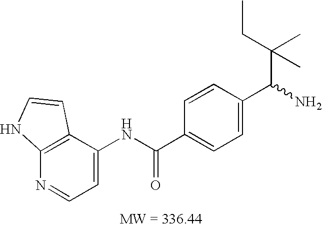

The title product is prepared according to the procedure of Compound 41, starting from 4-(1-benzyloxycarbonylamino-2,2-dimethyl-butyl)-benzoic acid and Intermediate 5.

### Compound 56: 1-amino-indan-5-carboxylic acid (1H-pyrrolo[2,3pyridinedin-4-yl)-amide

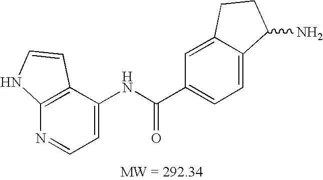

The title product is prepared according to the procedure of Compound 41, starting from 1-benzyloxycarbonylamino-indan-5-carboxylic acid and Intermediate 5.

### Compound 57: 5-amino-5,6,7,8-tetrahydro-naphthalene-2-carboxylic acid (1H-pyrrolo[2,3pyridinedin-4-yl)-amide

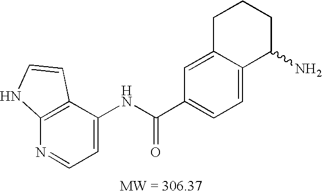

The title product is prepared according to the procedure of Compound 41, starting from 5-benzyloxycarbonylamino-5,6,7,8-tetrahydro-naphthalene-2-carboxylic acid and Intermediate 5.

### Compound 58: 4-(1-amino-butyl)-N-(1H-pyrrolo[2,3pyridinedin-4-yl)-benzamide

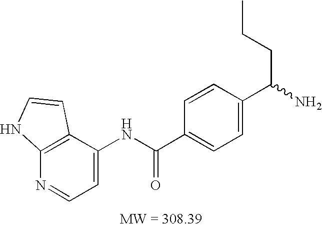

The title product is prepared according to the procedure of Compound 41, starting from 4-(1-benzyloxycarbonylamino-butyl)-benzoic acid and Intermediate 5.

### Compound 59: 4-(1-amino-2-methyl-propyl)-N-(1H-pyrrolo[2,3pyridinedin-4-yl)-benzamide

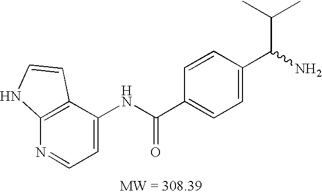

The title product is prepared according to the procedure of Compound 41, starting from 4-(1-benzyloxycarbonylamino-2-methyl-propyl)-benzoic acid and Intermediate 5.

### Compound 60: 4-(1-amino-2,2-dimethyl-propyl)-N-(1H-pyrrolo[2,3pyridinedin-4-yl)-benzamide

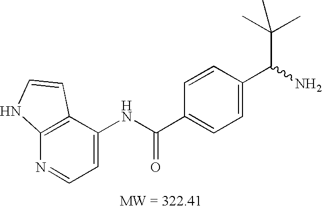

The title product is prepared according to the procedure of Compound 41, starting from 4-(1-benzyloxycarbonylamino-2,2-dimethyl-propyl)-benzoic acid and Intermediate 5.

### Example 4

### Biological Activity

Compounds 1-44 were tested for inhibition of the PKC isoforms PKC epsilon, PKC gamma, PKC theta and PKC zeta. Similarly, the compounds 45-60 are tested.

The inhibition assays were performed with a fluorescence polarization (FP) assay using the commercially available Protein Kinase C Assay Kit, Red, from Invitrogen (Product ID. No. 6905), essentially in accordance with the protocol supplied by the manufacturer. The substrate used was RFARKGSLRQKNV (M_{w} 1561), also obtained from Invitrogen (Product ID No. 6900). The isozymes PKC epsilon, PKC gamma, PKC theta and PKC zeta were also obtained from Invitrogen (Product ID Nos: 6906, 9343, 7101 and 9232).

In summary, all compounds were screened in the wells of a 384 well plate for inhibition of each of the isozymes with concentrations varying from 100 μM to 2 μM using a stepwise 2 (or 3)-fold dilution. Staurosporine was used as a reference (2 μM for PKC epsilon, gamma and theta and 40 μM for PKC zeta).

To perform the assay, 2 μl of a solution of the compound to be tested in DMSO (at each concentration) was added to 6 μl of a solution of the enzyme in 10 mM HEPES, 5 mM dithiotreitol, 0.1% Triton X-100, pH 7,4. The final concentration of the enzymes were 10 ng/ml for PKC epsilon and 20 ng/ml for PKC gamma, theta and zeta.

After incubating for 30 minutes at room temperature, 4 μl of a mixture of ATP and the protein substrate in 60 mM HEPES (pH7.4), 15 mM MgCl₂, 0.3 mM CaCl₂, 0.06% NP40 was added. The final concentration of the ATP was 2.5 μM and final concentration of protein substrate was 1 μM.

After incubating for 80 minutes at room temperature, 3 μl of a mix solution of 500 mM EDTA (stop solution) and the Rhodamine-based PKC Red Tracer (from the Protein Kinase C Assay Kit) in BGG/phosphate buffer (pH7.4) with 0.02% NaN₃ and 0.1% Triton X-100 was added and 5 μl of a the Anti-Phosphoserine antibody (also from the Protein Kinase C Assay Kit) in BGG/phosphate buffer (pH7.4) with 0.02% NaN₃.

The mixture thus obtained (total volume: 20 μl) was incubated for 60 minutes at room temperature, upon which the fluorescence polarization was measured using an automated plate reader (Perkin Elmer, Model Envision 2100-0010 HTS) with FP filters for rhodamine: excitation filter FITC FP 531 and emission filters FITC FP P-pol 595 and FITC FP S-pol 595 (Perkin-Elmer).

The results were fitted to a curve using the XL-Fit algorithm and IC₅₀ values were calculated for each fitted curve, again using the XL-Fit algorithm.

The results for the compounds tested are shown in the Table 1 below. Compounds 1 to 17 are comparative examples; Compounds 18 to 44 are examples of compounds of the invention. In Table 1, “MW” indicates the molecular weight, and “D” indicates the distance between the pyridine-nitrogen atom and the nitrogen atom in the amino group, as determined by Scatter Plot (as described above). For the compounds 2-4, no distance could be determined, as these compounds do not contain a pyridine-nitrogen.

The IC₅₀ values for the reference compound, staurosporine, were 0.045 μM for PKC epsilon, 0.02 μM for PKC gamma, 0.05 μM for PKC theta and 1 μM for PKC zeta.

Active compounds according to the present invention are compounds that have an IC₅₀ of less than 100 μM. The results demonstrate that several compounds that are active on PKCepsilon also active (as defined above) on PKCtheta. PKCtheta is an example of another interesting kinase which can mediate an insulin resistance in insulin target organs due to an impairment of insulin signalling pathway. Thus, inhibition of both kinases with a single compound proves an additional advantage over inhibition of each kinase independently.

TABLE 1

| | | IC₅₀ μM | | | |
|---|---|---|---|---|---|
| Compound | Formula | PKCε | PKCγ | PKCθ | PKCζ |
| 1 | 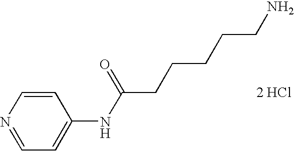 MW = 207.28(+2 HCl) D = 10.93 | >100 | >100 | >100 | >100 |
| 2 | 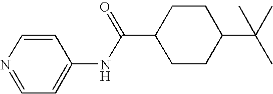 MW = 260.38 | >100 | >100 | >100 | >100 |
| 3 | 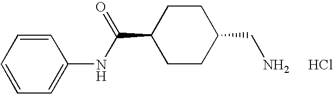 MW = 232.33(+HCl) | >100 | 90.5 | >100 | 40.3 |
| 4 | 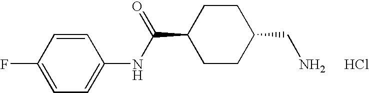 MW = 250.32(+HCl) | >100 | >100 | >100 | >100 |
| 5 | 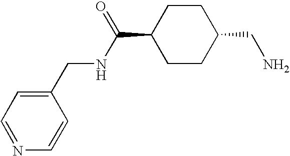 MW = 247.34 D = 11.76 | >100 | >100 | >100 | >100 |
| 6 | 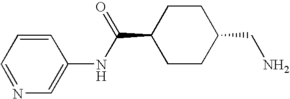 MW = 233.32 D = 6.75 | >100 | >100 | >100 | >100 |
| 7 | 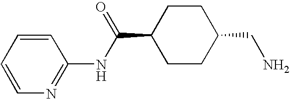 MW = 233.32 D = 8.82 | >100 | >100 | >100 | >100 |
| 8 | 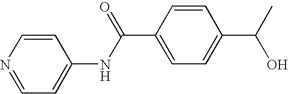 MW = 242.28 D = 11.07 | >100 | >100 | >100 | >100 |
| 9 | 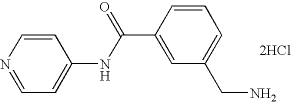 MW = 227.27(+2HCl) D = 10.87 | >100 | >100 | >100 | >100 |
| 10 | 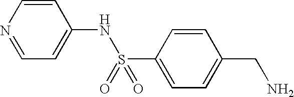 MW = 263.32 D = 6.06 | >100 | >100 | >100 | >100 |
| 11 | 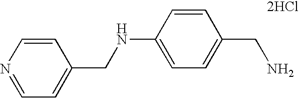 MW = 213.28(+2HCl) D = 10.87 | >100 | >100 | >100 | >100 |
| 12 | 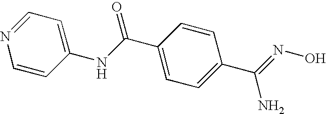 MW = 256.27 D = 11.10 | >100 | >100 | >100 | >100 |
| 13 | 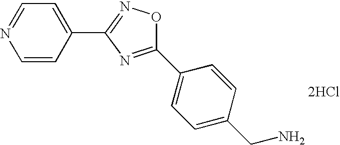 MW = 252.28(+2HCl) D = 11.77 | >100 | >100 | >100 | >100 |
| 14 | 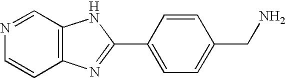 MW = 224.27 D = 10.52 | >100 | >100 | >100 | >100 |
| 15 | 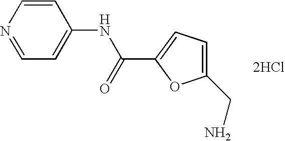 MW = 217.23(+2HCl) D = 9.15 | >100 | >100 | >100 | >100 |
| 16 | 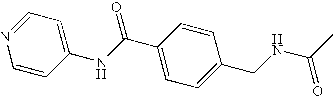 MW = 269.31 D = 11.11 | >100 | >100 | >100 | >100 |
| 17 | 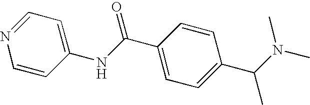 MW = 269.35 D = 11.40 | >100 | >100 | >100 | >100 |
| 18 | 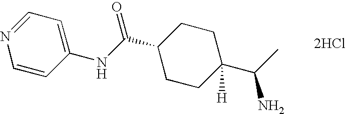 MW = 247.34(+2HCl) Y-27632 dihydrochloride D = 11.12 | 1.74 | >100 | 1.73 | 37.68 |
| 19 | 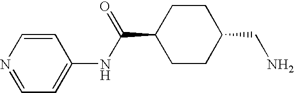 MW = 233.32 D = 11.16 | 5.12 | >100 | 7.35 | 24.33 |
| 20 | 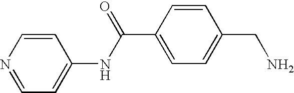 MW = 227.27 D = 11.13 | 6.23 | >100 | 9.14 | 51.40 |
| 21 | 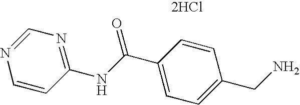 MW = 228.26(+2HCl) D = 11.01 | 34.41 | >100 | 87.18 | >100 |
| 22 | 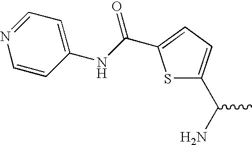 MW = 247.32 D = 10.94 | 16 | >100 | 9.01 | 45.9 |
| 23 | 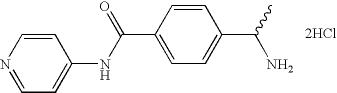 MW = 241.30(+2HCl) D = 11.16 | 1.21 | >100 | 2.25 | 22.14 |
| 24 | 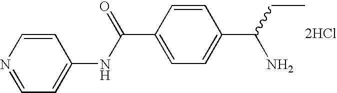 MW = 255.32(+2HCl) D = 11.53 | 0.65 | >30 | 2.91 | 25.4 |
| 25 | 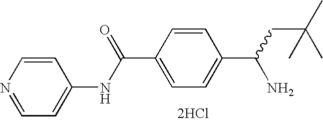 MW = 297.40(+2HCl) D = 11.51 | 4.98 | >100 | 12.18 | >100 |
| 26 | 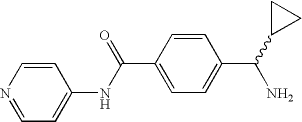 MW = 267.33 D = 11.58 | 0.72 | 35.9 | 3.9 | 41.5 |
| 27 | 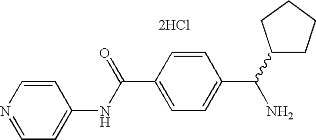 MW = 295.39(+2HCl) D = 11.53 | 0.94 | 76 | 1.7 | 70 |
| 28 | 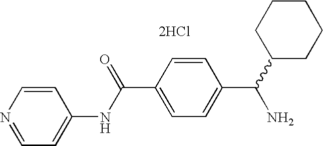 MW = 309.41(+2HCl) D = 11.38 | 2.6 | >100 | 7.7 | >100 |
| 29 | 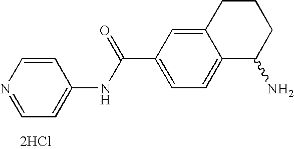 MW = 267.33(+2HCl) D = 11.39 | 6.1 | >100 | 13.7 | >100 |
| 30 | 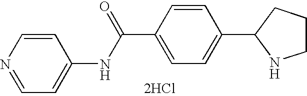 MW = 267.33(+2HCl) D = 11.43 | 9.8 | >100 | 17.2 | >100 |
| 31 | 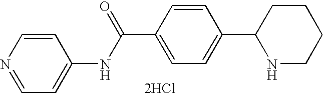 MW = 281.36(+2HCl) D = 11.41 | 3.7 | >100 | 8.04 | >100 |
| 32 | 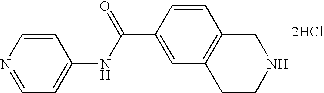 MW = 253.31(+2HCl) D = 11.64 | 30.5 | >100 | 31.6 | >100 |
| 33 | 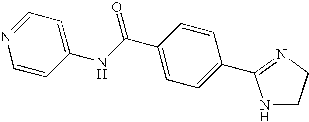 MW = 266.31 D = 11.64 | 61.97 | >100 | 53.48 | >100 |
| 34 | 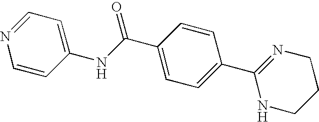 MW = 280.33 D = 11.12 | 19.5 | >100 | 75 | >100 |
| 35 | 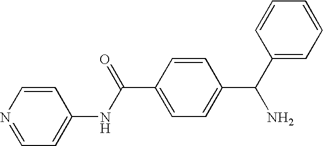 MW = 303.37 D = 11.70 | 8.04 | >100 | 11.3 | >100 |
| 36 | 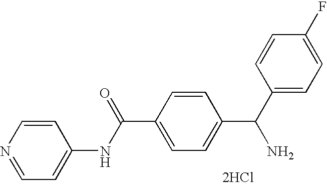 MW = 321.36(+2HCl) D = 11.70 | 9.5 | >100 | 28.9 | >100 |
| 37 | 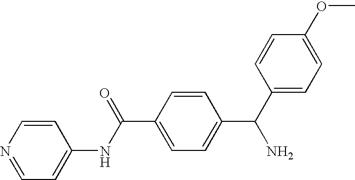 MW = 333.39 D = 11.43 | 18.7 | >100 | 80.3 | >100 |
| 38 | 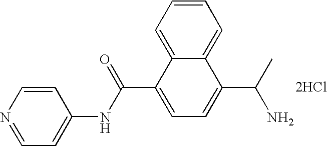 MW = 291.36(+2HCl) D = 11.56 | 2.5 | >100 | 7.8 | 6.4 |
| 39 | 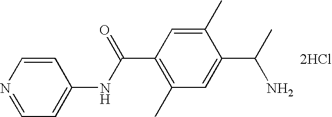 MW = 255.32(+2HCl) D = 11.52 | 8.3 | >100 | 16.8 | >100 |
| 40 | 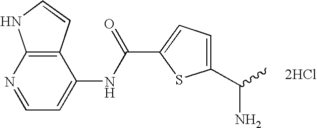 MW = 286.36(+2HCl) D = 10.74 | 3.9 | 30 | 3.2 | >100 |
| 41 | 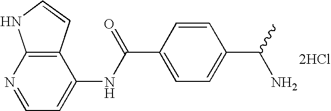 MW = 280.33(+2HCl) D = 11.21 | 0.40 | 11.29 | 0.77 | >100 |
| 42 | 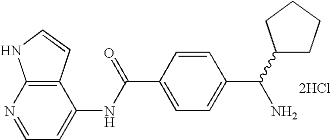 MW = 334.42(+2HCl) D = 11.44 | 0.3 | 17.3 | 0.63 | >100 |
| 43 | 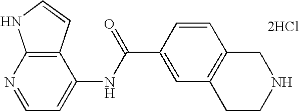 MW = 292.34(+2HCl) D = 11.70 | 9.9 | 21.8 | 8.6 | >100 |
| 44 | 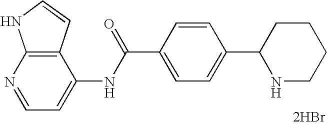 MW = 292.34(+2HBr) D = 11.58 | 1.6 | 30.7 | 2.2 | >100 |

All patents, patent applications, and published references cited herein are hereby incorporated by reference in their entirety. While this invention has been particularly shown and described with references to preferred embodiments, it will be understood by those skilled in the art that various changes in form and details may be made without departing from the scope of the invention encompassed by the claims.

1.-31. (canceled)

32. A compound according to formula I: 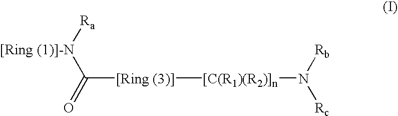
wherein
n is 1;
Ring (1) is of formula 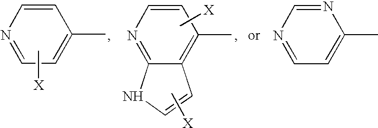
wherein —X may be absent or denotes substitution with 1-4 substitutents X that are independently chosen from halogen, C₁-C₆ alkyl, C₁-C₆ alkoxy, substituted or unsubstituted aryl, nitro, hydroxyl and a substituted or unsubstituted amino group;
Ring (3) is a 1,3-phenylene, 1,4-phenylene, 1,3-cyclohexylene, or 1,4-cyclohexylene optionally substituted with 1-4 substitutents that are independently selected from halogen, C₁-C₆ alkyl, C₁-C₆ alkoxy, substituted or unsubstituted aryl, nitro, hydroxyl, an amino group;
Rₐ is hydrogen; a linear or branched, optionally substituted C₁-C₆-alkyl; a linear or branched, optionally substituted C₁-C₆-alkoxy; or an optionally substituted aryl; 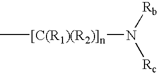
represents a group selected from 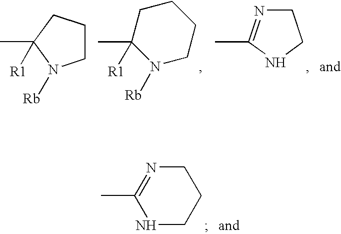
R₁ is selected from the group consisting of hydrogen; a substituted or unsubstituted, saturated, unsaturated or aromatic 3-, 4-, 5-, 6-, 7-or 8-membered ring containing carbon atoms and optionally one or two heteroatoms; substituted or unsubstituted C₁-C₆ alkyl and cyano,
or a salt, pharmaceutically acceptable salt, pharmaceutically acceptable prodrug, tautomer, isomer, and/or stereochemical isomer thereof.

33. The compound according to claim 32, wherein
Ring (1) is of formula 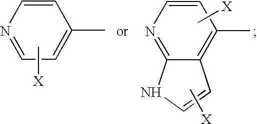
Ring (3) is of formula 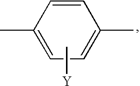
wherein —Y may be absent or denotes substitution with 1-4 substitutents Y that are independently chosen from halogen, C₁-C₆ alkyl, C₁-C₆ alkoxy, substituted or unsubstituted aryl, nitro, hydroxyl, and an amino group; and 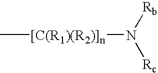
represents a group that is

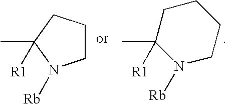

34. The compound according to claim 33, wherein
Ring (1) is of formula 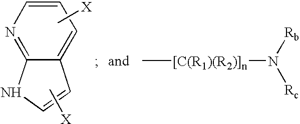
represents a group that is

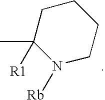

35. The compound according to claim 34, wherein X denotes substitution with 1 or 2 substitutents X.

36. The compound according to claim 35, wherein —Y denotes substitution with 1 or 2 substitutents Y.

37. A compound selected from the group consisting of
N-pyridin-4-yl-4-pyrrolidin-2-yl-benzamide;
4-piperidin-2-yl-N-pyridin-4-yl-benzamide;
1,2,3,4-tetrahydro-isoquinoline-6-carboxylic acid pyridin-4-yl-amide;
4-(4,5-dihydro-1H-imidazol-2-yl)-N-pyridin-4-yl-benzamide;
N-pyridin-4-yl-4-(1,4,5,6-tetrahydro-1H-pyrimidin-2-yl)-benzamide;
4-(1-amino-phenyl-methyl)-N-pyridin-4-yl-benzamide;
4-[1-amino-(4-fluorophenyl)-methyl]-N-pyridin-4-yl-benzamide;
4-[1-amino-(4-methoxyphenyl)-methyl]-N-pyridin-4-yl-benzamide;
4-(1-amino-ethyl)-naphthalene-1-carboxylic acid pyridin-4-ylamide;
4-aminomethyl-2,5-dimethyl-N-pyridin-4-yl-benzamide;
4-(1-amino-ethyl)-N-(1H-pyrrolo[2,3-b]pyridin-4-yl)-benzamide;
4-(1-amino-cyclopentyl-ethyl)-N-(1H-pyrrolo[2,3-b]pyridin-4-yl)-benzamide;
1,2,3,4-tetrahydro-isoquinoline-6-carboxylic acid-N (1H pyrrolo[2,3-b]pyridin-4-yl)-benzamide;
4-piperidin-2-yl-N-(1H-pyrrolo[2,3-b]pyridin-4-yl)-benzamide;
4-(1-amino-cyclobutyl-ethyl)-N-pyridin-4-yl-benzamide;
4-(1-amino-2,2-dimethyl-butyl)-N-pyridin-4-yl-benzamide;
1-amino-indan-5-carboxylic acid pyridin-4-yl-amide;
4-(1-amino-butyl)-N-pyridin-4-yl-benzamide;
4-(1-amino-pentyl)-N-pyridin-4-yl-benzamide;
4-(1-amino-2-methyl-propyl)-N-pyridin-4-yl-benzamide;
4-(1-amino-2,2-dimethyl-propyl)-N-pyridin-4-yl-benzamide;
4-(1-amino-propyl)-N-(1H-pyrrolo[2,3-b]pyridin-4-yl)-benzamide;
4-(1-amino-cyclopropyl-ethyl)-N-(1H-pyrrolo[2,3-b]pyridin-4-yl)-benzamide;
4-(1-amino-cyclobutyl-ethyl)-N-(1H-pyrrolo[2,3-b]pyridin-4-yl)-benzamide;
4-(1-amino-2,2-dimethyl-butyl)-N-(1H-pyrrolo[2,3-b]pyridin-4-yl)-benzamide;
1-amino-indan-5-carboxylic acid(1H-pyrrolo[2,3-b]pyridin-4-yl)-amide;
5-amino-5,6,7,8-tetrahydro-naphthalene-2-carboxylic acid (1H-pyrrolo[2,3-b]pyridin-4-yl)-amide;
4-(1-amino-butyl)-N-(1H-pyrrolo[2,3-b]pyridin-4-yl)-benzamide;
4-(1-amino-2,2-dimethyl-propyl)-N-(1H-pyrrolo[2,3-b]pyridin-4-yl)-benzamide; and
or salt, pharmaceutically acceptable salt, pharmaceutically acceptable prodrug, tautomer, isomer, and/or stereochemical isomer thereof.

38. A pharmaceutical composition comprising a therapeutically effective amount of a compound according to claim 32 and a pharmaceutically acceptable carrier, diluent, excipient, and/or adjuvant.

39. A method for the treatment or prevention of a metabolic disease or disorder, or complications and/or symptoms thereof, in a mammal, comprising administering to the mammal a therapeutically effective amount of a compound according to the following formula, or a salt or prodrug thereof: 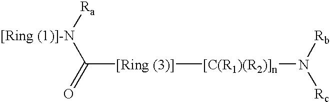
wherein
Ring (1) is a substituted or unsubstituted, saturated, unsaturated or aromatic 4-, 5-, 6-, 7-, or 8-membered ring containing carbon atoms and at least one hydrogen-accepting heteroatom and optionally 1 or 2 further heteroatoms;
Rₐ is a hydrogen or a linear or branched, substituted or unsubstituted C₁-C₆ alkyl, substituted or unsubstituted C₁-C₆ alkoxy or substituted or unsubstituted aryl;
Ring (3) is a substituted or unsubstituted, saturated, unsaturated or aromatic 4-, 5-, 6-, 7-, or 8-membered ring containing carbon atoms and optionally 1 or 2 further heteroatoms;
each R₁ or R₂ may be the same or different and is independently selected from the group consisting of hydrogen, a substituted or unsubstituted, saturated, unsaturated or aromatic 3-, 4-, 5-, 6-, 7-or 8-membered ring containing carbon atoms and optionally one or two heteroatoms, substituted or unsubstituted C₁-C₆ alkyl or cyano;
n is 0, 1, or 2; and
R_{b} and R_{c} are such that the amino group —NR_{b}R_{c} is essentially in a protonated form at a pH between 5.0-9.0;
and wherein
(1) the group Rₐ, the nitrogen atom to which group Rₐ is bound, the carbon atom of Ring (1) to which the N—Rₐ nitrogen atom is bound, and one carbon atom of Ring (1) adjacent to the carbon atom of Ring (1) to which the N—Rₐ nitrogen atom is bound may form Ring (7) wherein Ring (7) is a substituted or unsubstituted, saturated, unsaturated or aromatic 4-, 5-, or 6-membered ring that contains carbon atoms, the N—Rₐ nitrogen atom and optionally one further heteroatom chose from oxygen, sulfur, and nitrogen;
(2) where Ring (3) is a 1,4-phenylene group, one of R₁ and R₂, the carbon atom to which R₁ and R₂ are bound and two of the carbon atoms belonging to the 1,4-phenylene group may form a substituted or unsubstituted 5-, 6-, 7-, or 8-membered ring that contains carbon atoms, the nitrogen atom of the amino group NR_{b}R_{c} and optionally one further heteroatom chosen from oxygen, sulfur, and nitrogen and that may be saturated or contain one double bond;
(3) where Ring (3) is a 1,4-phenylene group, one of R_{b} or R_{c}, the nitrogen atom to which R_{b} or R_{c} are bound, the carbon atom to which R₁ or R₂ are bound and two of the carbon atoms belonging to the 1,4-phenylene group may form a substituted or unsubstituted 5-, 6-, 7-, or 8-membered ring that contains carbon atoms, the nitrogen atom of the amino group NR_{b}R_{c} and optionally one further heteroatom chosen from oxygen, sulfur, and nitrogen and that may be saturated or contain one double bond;
(4) one of R_{b} and R_{c} may, together with the nitrogen atom of the amino group NR_{b}R_{c}, one of R₁ and R₂ and the carbon atom to which R₁ and R₂ are bound, form a substituted or unsubstituted 5-, 6-, 7-, or 8-membered ring that contains carbon atoms, the nitrogen atom of the amino group NR_{b}R_{c} and optionally one further heteroatom chosen from oxygen, sulfur, and nitrogen and that may be saturated or contain one double bond;
(5) R_{b}, R_{c} and the nitrogen atom to which they are bound may together form a substituted or unsubstituted ring with between 3 and 10 atoms in the ring, including the nitrogen atom to which both Rₐ and R_{b} are bound, so that the ring so formed consists of a nitrogen atom, carbon atoms and optionally one further heteroatom chose from oxygen, nitrogen, and sulfur;
and wherein
the distance between the at least one hydrogen-accepting heteroatom in Ring (1) and the NRₐR_{b} nitrogen atom, as determined using a Scatter Plot, is in the range of 11.0 to 11.8 Angstroms.

40. The method according to claim 39, wherein the disease or disorder is selected from the group consisting of hyperglycemia, hyperinsulinemia, hyperlipidemia, and insulin-resistant diabetes, lipoatrophies, and obesity.

41. The method according to claim 39, wherein the disease or disorder is selected from the group consisting of conditions and/or diseases that are primarily associated with the response or sensitivity to insulin,

42. The method according to claim 39, wherein the disease or disorder is selected from the group consisting of Type I and Type II diabetes, severe insulin resistance, Mendenhall's Syndrome, Werner Syndrome, leprechaunism, lipoatrophic diabetes, hypertension, osteoporosis and lipodystrophy.

43. The method according to claim 39, wherein the disease or disorder is Type II diabetes, or a complication or symptom associated therewith.

44. The method according to claim 39, wherein the disease or disorder is obesity, or a complication or symptom associated therewith.

45. The method according to claim 39, wherein
Ring (1) is of formula 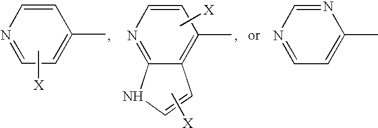
wherein —X may be absent or denotes substitution with 1-4 substitutents X that are independently chosen from halogen, C₁-C₆ alkyl, C₁-C₆ alkoxy, substituted or unsubstituted aryl, nitro, hydroxyl and a substituted or unsubstituted amino group,
Rₐ is a hydrogen;
Ring (3) is of formula 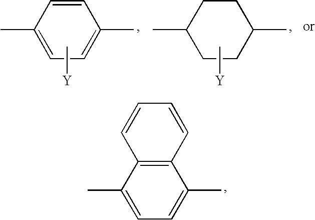
wherein —Y may be absent or denotes substitution with 1-4 substitutents Y that are independently chosen from halogen, C₁-C₆ alkyl, C₁-C₆ alkoxy, substituted or unsubstituted aryl, nitro, hydroxyl and an amino group,
each R₁ or R₂, may be the same or different, and is independently selected from the group consisting of hydrogen, a substituted or unsubstituted, saturated, unsaturated or aromatic 3-, 4-, 5-, 6-, 7-or 8-membered ring containing carbon atoms and optionally one or two heteroatoms, substituted or unsubstituted C₁-C₆ alkyl or cyano; n is 1; and R_{b} and R_{c} are each independently hydrogen; or 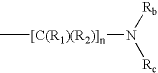
is of formula 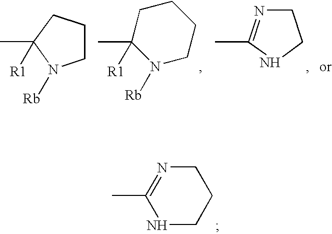
or wherein: 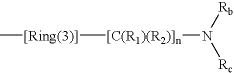
is of formula

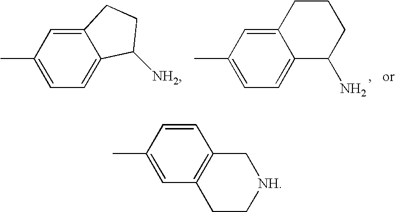

46. A method for inhibition of the activity of at least one kinase, comprising contacting said kinase with a compound according to the following formula, or a salt or prodrug thereof: 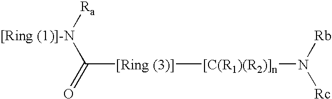
wherein
Ring (1) is a substituted or unsubstituted, saturated, unsaturated or aromatic 4-, 5-, 6-, 7-, or 8-membered ring containing carbon atoms and at least one hydrogen-accepting heteroatom and optionally 1 or 2 further heteroatoms;
Rₐ is a hydrogen or a linear or branched, substituted or unsubstituted C₁-C₆ alkyl, substituted or unsubstituted C₁-C₆ alkoxy or substituted or unsubstituted aryl;
Ring (3) is a substituted or unsubstituted, saturated, unsaturated or aromatic 4-, 5-, 6-, 7-, or 8-membered ring containing carbon atoms and optionally 1 or 2 further heteroatoms;
each R₁ or R₂ may be the same or different and is independently selected from the group consisting of hydrogen, a substituted or unsubstituted, saturated, unsaturated or aromatic 3-, 4-, 5-, 6-, 7-or 8-membered ring containing carbon atoms and optionally one or two heteroatoms, substituted or unsubstituted C₁-C₆ alkyl or cyano;
n is 0, 1, or 2; and
R_{b} and R_{c} are such that the amino group —NR_{b}R_{c} is essentially in a protonated form at a pH between 5.0-9.0;
and wherein
(1) the group Rₐ, the nitrogen atom to which group Rₐ is bound, the carbon atom of Ring (1) to which the N—Rₐ nitrogen atom is bound, and one carbon atom of Ring (1) adjacent to the carbon atom of Ring (1) to which the N—Rₐ nitrogen atom is bound may form Ring (7) wherein Ring (7) is a substituted or unsubstituted, saturated, unsaturated or aromatic 4-, 5-, or 6-membered ring that contains carbon atoms, the N—Rₐ nitrogen atom and optionally one further heteroatom chose from oxygen, sulfur, and nitrogen;
(2) where Ring (3) is a 1,4-phenylene group, one of R₁ and R₂, the carbon atom to which R₁ and R₂ are bound and two of the carbon atoms belonging to the 1,4-phenylene group may form a substituted or unsubstituted 5-, 6-, 7-, or 8-membered ring that contains carbon atoms, the nitrogen atom of the amino group NR_{b}R_{c} and optionally one further heteroatom chosen from oxygen, sulfur, and nitrogen and that may be saturated or contain one double bond;
(3) where Ring (3) is a 1,4-phenylene group, one of R_{b} or R_{c}, the nitrogen atom to which R_{b} or R_{c} are bound, the carbon atom to which R₁ or R₂ are bound and two of the carbon atoms belonging to the 1,4-phenylene group may form a substituted or unsubstituted 5-, 6-, 7-, or 8-membered ring that contains carbon atoms, the nitrogen atom of the amino group NR_{b}R_{c} and optionally one further heteroatom chosen from oxygen, sulfur, and nitrogen and that may be saturated or contain one double bond;
(4) one of R_{b} and R_{c} may, together with the nitrogen atom of the amino group NR_{b}R_{c}, one of R₁ and R₂ and the carbon atom to which R₁ and R₂ are bound, form a substituted or unsubstituted 5-, 6-, 7-, or 8-membered ring that contains carbon atoms, the nitrogen atom of the amino group NR_{b}R_{c} and optionally one further heteroatom chosen from oxygen, sulfur, and nitrogen and that may be saturated or contain one double bond;
(5) R_{b}, R_{c} and the nitrogen atom to which they are bound may together form a substituted or unsubstituted ring with between 3 and 10 atoms in the ring, including the nitrogen atom to which both Rₐ and R_{b} are bound, so that the ring so formed consists of a nitrogen atom, carbon atoms and optionally one further heteroatom chose from oxygen, nitrogen, and sulfur;
and wherein
the distance between the at least one hydrogen-accepting heteroatom in Ring (1) and the NRₐR_{b} nitrogen atom, as determined using a Scatter Plot, is in the range of 11.0 to 11.8 Angstroms.

47. The method according to claim 46, wherein the inhibition is in vivo.

48. The method according to claim 46, wherein the inhibition is in vitro.

49. The method according to claim 46, wherein
Ring (1) is of formula 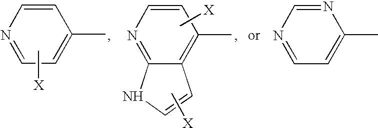
wherein —X may be absent or denotes substitution with 1-4 substitutents X that are independently chosen from halogen, C₁-C₆ alkyl, C₁-C₆ alkoxy, substituted or unsubstituted aryl, nitro, hydroxyl and a substituted or unsubstituted amino group,
Rₐ is a hydrogen;
Ring (3) is of formula 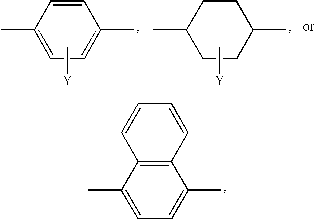
wherein —Y may be absent or denotes substitution with 1-4 substitutents Y that are independently chosen from halogen, C₁-C₆ alkyl, C₁-C₆ alkoxy, substituted or unsubstituted aryl, nitro, hydroxyl and an amino group,
each R₁ or R₂, may be the same or different, and is independently selected from the group consisting of hydrogen, a substituted or unsubstituted, saturated, unsaturated or aromatic 3-, 4-, 5-, 6-, 7-or 8-membered ring containing carbon atoms and optionally one or two heteroatoms, substituted or unsubstituted C₁-C₆ alkyl or cyano; n is 1; and R_{b} and R_{c} are each independently hydrogen; or 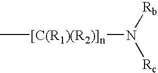
is of formula 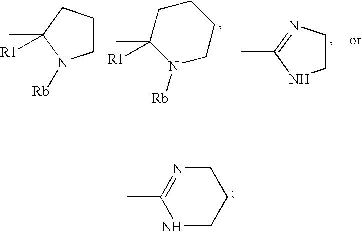
or wherein: 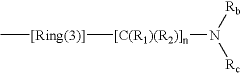
is of formula [chemical structure]
